(19) United States
(12) Patent Application Publication
(10) Pub. No.: US 2026/0234662 A1
(43) Pub. Date: Aug. 13, 2026

(54) Nucleic Acids Encoding Soluble PD-1 and IL-12 and Uses Thereof

(71) Applicant: KaliVir Immunotherapeutics, Inc., Pittsburgh, PA (US)

(72) Inventors: Stephen Howard THORNE, Pittsburgh, PA (US); Mingrui ZHANG, Pittsburgh, PA (US)

(21) Appl. No.: 19/181,926

(22) Filed: Apr. 17, 2025

Related U.S. Application Data
(63) Continuation of application No. PCT/US2023/077137, filed on Oct. 18, 2023.
(60) Provisional application No. 63/417,484, filed on Oct. 19, 2022.

(51) Int. Cl. C12N 15/86; A61K 38/17; A61K 38/20; A61K 48/00; A61P 35/00; C07K 14/54; C07K 14/705; C07K 14/715

(52) U.S. Cl. CPC C12N 15/86; A61K 38/1774; A61K 38/1793; A61K 38/208; A61K 48/005; A61P 35/00; C07K 14/5434; C07K 14/70521; C07K 14/7158; C12N 2710/24132; C12N 2710/24143

(57) ABSTRACT

The present disclosure provides for nucleic acids encoding for a soluble PD-1 variant and IL-12. Further provided herein are nucleic acids encoding for a chemokine receptor. Additionally described herein are oncolytic viruses comprising the nucleic acids described herein. Oncolytic viruses optionally comprise a mutation or deletion of the gene expressing IFN-gamma. Compositions described herein are further described for use in the treatment of cancer.

### CROSS-REFERENCE

This application is a continuation of PCT Application No. PCT/US2023/077137, filed Oct. 18, 2023, which claims the benefit of U.S. Provisional Application No. 63/417,484 filed Oct. 19, 2022, which are incorporated by reference herein in their entirety.

### SEQUENCE LISTING

The instant application contains a Sequence Listing which has been submitted electronically in ST.26 xml format and is hereby incorporated by reference in its entirety. Said xml copy, created on Sep. 28, 2023, is named 199249-725601_SL.xml and is 112,890 bytes in size.

### BACKGROUND

Tumor cells express Programmed Cell Death Protein 1 ligand (PD-L1) to drive checkpoint inhibition and evade immune response. PD-L1 binds PD-1 on T cells in a checkpoint to suppress a T cell response. Blocking PD-L1 can enhance T cell function in a cancer therapy. A truncated PD-1 lacking the transmembrane domain provides for a soluble PD-1. Soluble PD-1 can act as a decoy receptor to bind PD-L1 on tumor cells, blocking the PD-L1: PD-1 signaling pathway, acting as a checkpoint inhibitor. Described herein are improved cancer therapies incorporating checkpoint inhibitor interventions.

IL-12 enhances an immune response by activating T-cells and NK cells. Described herein is use of a soluble PD-1 in conjunction with IL-12 for the treatment of cancer.

### BRIEF SUMMARY

Described herein are nucleic acids, wherein the nucleic acid comprises a sequence encoding for at least two polypeptides, wherein the at least two polypeptides comprise: interleukin-12 (IL-12) or a functional variant thereof; and soluble PD-1 (sPD-1) or a functional variant thereof.

Described herein are nucleic acids, wherein the nucleic acid comprises: a first region encoding a first polypeptide comprising a sequence having at least 85%, 90%, 95%, or 99% sequence identity to SEQ ID NO: 5 or SEQ ID NO: 8; and a second region encoding a second polypeptide comprising a sequence having at least 85%, 90%, 95%, or 99% sequence identity to SEQ ID NO: 2 or SEQ ID NO: 4.

Described herein are compositions, wherein the composition comprises: a vector; an exogenous nucleic acid comprising a sequence encoding for a cytokine or a functional variant thereof; and an exogenous nucleic acid comprising a sequence encoding for a PD-L1 receptor.

Described herein are oncolytic viruses, wherein the oncolytic virus comprises: an insertion at a TK gene locus comprising, in 5′ to 3′ order: a first promoter region, wherein the promoter is P7.5; a first region encoding IL-12; a second promoter region, wherein the promoter is P28; and a second region encoding a PD-1 variant.

Described herein are pharmaceutical compositions, wherein the pharmaceutical composition comprises: the nucleic acid of as described herein or the composition as described herein; and a pharmaceutically acceptable excipient.

Described herein are methods for treatment of cancer comprising administering to a subject having cancer the pharmaceutical composition of as described herein in an amount sufficient for treatment of a cancer.

Described herein are methods for activating an anti-tumor immune response, comprising administering to a subject having a cancer the pharmaceutical composition as described herein.

Described herein are methods for reduction of incidence of tumor cell growth, comprising: administering to tumor cells the pharmaceutical composition as described herein in an effective amount sufficient for reduction of incidence of tumor cell growth.

### BRIEF DESCRIPTION OF THE DRAWINGS

The novel features of the disclosure are set forth with particularity in the appended claims. A better understanding of the features and advantages of this disclosure will be obtained by reference to the following detailed description that sets forth illustrative embodiments, in which the principles of this disclosure are utilized, and the accompanying drawings of which:

FIG. 1A is a schematic representation of the PD-1/PD-L1 interaction between a tumor cell and T cell.

FIG. 1B is a schematic representation of sPD-1 binding to PD-L1 on a T cell, preventing the PD-1/PD-L1 interaction.

FIG. 2 is a diagram of a transgenes inserted in the TK locus with a P7.5 promoter driving expression of a gene encoding an IL-12 polypeptide, comprising IL-12 beta and alpha subunits linked by a 22-residue glycine-rich linker and a P28 promoter driving expression of a gene encoding soluble PD-1 polypeptide.

FIG. 3A is a graph plotting volume of induced Renca cell tumors in mice 45 days post treatment on the y-axis in treatment groups as shown on the x-axis; treatment groups received buffer control, TK-control, or TK-virus modified to express murine IL-12 and murine sPD-1. Control group tumors measured at least 1000 mm³ and treatment group average size was about 200 mm³. Treatment group receiving modified virus showed a Complete Response (CR) of 40%.

FIG. 3B is a graph plotting volume of induced B16 cell tumors in mice 38 days post treatment on the y-axis in treatment groups as shown on the x-axis; treatment groups received buffer control, TK-control, or TK-virus modified to express murine IL-12 and murine sPD-1. Control group tumors measured at least 1400 mm³ and treatment group average size was below detectable levels. Treatment group receiving modified virus shows a CR of 90%.

FIG. 4 is a graph plotting volume of LLC tumors in mice 31 days post treatment on the y-axis in treatment groups as shown on the x-axis; treatment groups received buffer control, TK-control, TK-/B8R-control, or TK-/BR8-virus modified to express murine IL-12 and murine SPD-1. Buffer and TK-control group tumors measured at least 1400 mm³. TK and treatment group average size was below detectable levels. Treatment group receiving modified virus showed a CR of 80%.

FIG. 5 is a diagram of a transgene inserted in the A52R locus using the A52R promoter to drive expression of a CXCR3 chemokine receptor.

### DETAILED DESCRIPTION

Tumor cells employ various mechanisms to avoid detection attack by the host immune system. Such mechanisms can influence the effectiveness of a cancer immunotherapy. Described herein are compositions comprising a combination of immune checkpoint inhibitor and pro-inflammatory cytokine, in order to enhance an immune response to tumor cells, either alone or in conjunction with other therapeutic modalities.

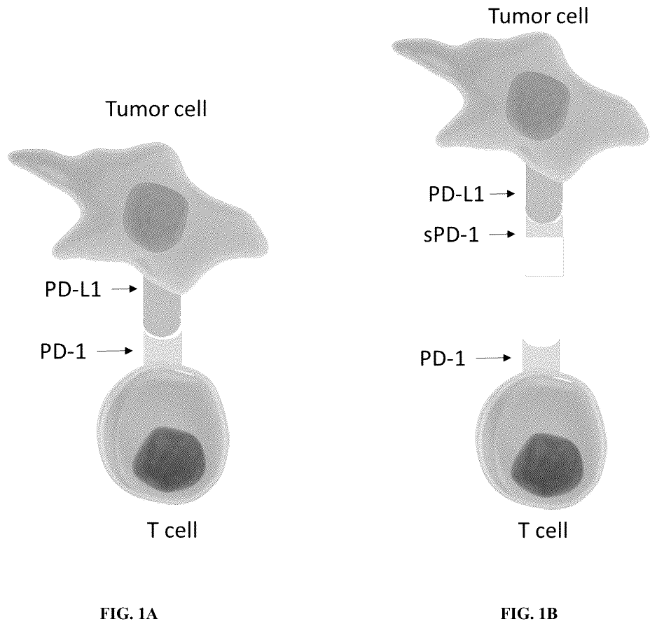

Programmed cell death protein 1 (PD-1) is an immune checkpoint protein expressed on the surface of T and B cells. When bound to ligand PD-L1, found on, for example, macrophages, the T-cell response is suppressed. The protein aids in regulating autoimmunity by suppressing a T cell inflammatory response to “self” cells. Some tumor cells leverage this immunosuppression by expressing PD-L1 on their surface (FIG. 1A). Binding of PD-L1 on tumor cells with PD-1 on T cells downregulates T cell activity and suppresses anti-tumor activity. Inhibition of the PD-L1/PD-1 interaction, or immune checkpoint inhibition, can enhance T-cell responses and increase anti-tumor activity. Described herein is a modified PD-1 protein lacking transmembrane and cytoplasmic domains, resulting in a soluble variant of PD-1 (SPD-1). An sPD-1 can bind with PD-L1, preventing the PD-L1/PD-1 checkpoint inhibition (FIG. 1B), allowing the T-cell response to proceed. In some embodiments, the sPD-1 described herein has maintained binding activity to PD-L1. Further described herein are nucleic acids encoding for sPD-1, and vectors comprising such nucleic acids.

IL-12 activates an anti-tumor cytotoxic immune response, modulating T cell, NK cell, and antigen-presenting cell responses. The cytokine enhances anti-tumor immune response and inhibits immunosuppression. Provided herein are compositions comprising nucleic acids encoding for co-expression of sPD-1 and IL-12.

Provided herein are compositions and uses thereof for treatment of cancer. Compositions described herein can comprise one or more nucleic acids encoding for polypeptides as described herein. Nucleic acids provided herein can comprise DNA, RNA, nucleic acid analogues, or any combination thereof. Briefly, described herein are (1) nucleic acids encoding for expression of SPD-1 and IL-12, (2) nucleic acids encoding for expression of chemokine receptors (3) vectors for expression of described nucleic acids, (4) modified oncolytic viruses, (5) conditions for treatment, and (6) dosage amounts, forms, and methods of administration of compositions described herein.

### Definitions

The terminology used herein is for the purpose of describing particular cases only and is not intended to be limiting. As used herein, the singular forms “a”, “an” and “the” include the plural forms as well, unless the context clearly indicates otherwise. Furthermore, to the extent that the terms “contains,” “containing,” “including”, “includes,” “having,” “has”, “with”, or variants thereof are used in either the detailed description and/or the claims, such terms are intended to be inclusive in a manner similar to the term “comprising.”

The term “about” or “approximately” can mean within an acceptable error range for the particular value as determined by one of ordinary skill in the art, which will depend in part on how the value is measured or determined, e.g., the limitations of the measurement system. Where particular values are described in the application and claims, unless otherwise stated the term “about” should be assumed to mean an acceptable error range for the particular value, such as ±10% of the value modified by the term “about”.

The terms “heterologous nucleic acid sequence,” or “exogenous nucleic acid sequence,” or “transgenes,” as used herein, in relation to a specific virus can refer to a nucleic acid sequence that originates from a source other than the specified virus.

The term “mutation,” as used herein, can refer to a deletion, an insertion of a heterologous nucleic acid, an inversion, or a substitution, including an open reading frame ablating mutations as commonly understood in the art.

The term “gene,” as used herein, can refer to a segment of nucleic acid that encodes for an individual protein or RNA (also referred to as a “coding sequence” or “coding region”), optionally together with associated regulatory regions such as promoters, operators, terminators, and the like, which may be located upstream or downstream of the coding sequence.

A “promoter,” as used herein, can be a control sequence that is a region of a nucleic acid sequence at which initiation and rate of transcription are controlled. In certain embodiments, a promoter may contain genetic elements at which regulatory proteins and molecules may bind such as RNA polymerase and other transcription factors. The terms “operatively positioned,” “operatively linked,” “under control” and “under transcriptional control” can mean that a promoter is in a correct functional location and/or orientation in relation to a nucleic acid sequence to control transcriptional initiation and/or expression of that sequence. In certain embodiments, a promoter may or may not be used in conjunction with an “enhancer,” which refers to a cis-acting regulatory sequence involved in the transcriptional activation of a nucleic acid sequence.

The term “homology,” as used herein, may be to calculations of “homology” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=# of identical positions/total # of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The homology can be between the entire lengths of two sequences or between fractions of the entire lengths of two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA.

The term “subject” can refer to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The terms “treat,” “treating,” and “treatment” can be meant to include alleviating or abrogating a disorder, disease, or condition; or one or more of the symptoms associated with the disorder, disease, or condition; or alleviating or eradicating the cause(s) of the disorder, disease, or condition itself.

The term “therapeutically effective amount” can refer to the amount of a compound that, when administered, can be sufficient to prevent development of, or alleviate to some extent, one or more of the symptoms of the disorder, disease, or condition being treated.

The term “oncolytic,” as used herein, can refer to killing of cancer or tumor cells by an agent, such as an oncolytic poxvirus, such as an oncolytic vaccinia virus, e.g., through the direct lysis of said cells, by stimulating immune response towards said cells, apoptosis, expression of toxic proteins, autophagy and shutdown of protein synthesis, induction of anti-tumoral immunity, or any combinations thereof. The direct lysis of the cancer or tumor cells infected by the agent, such as an oncolytic vaccinia virus, can be a result of replication of the virus within said cells. In certain examples, the term “oncolytic,” can refer to killing of cancer or tumor cells without lysis of said cells.

The term “oncolytic virus” as used herein can refer to a virus that preferentially infects and kills tumor cells. In some embodiments, the oncolytic viruses can include, but are not limited to, (i) viruses that naturally replicate preferentially in cancer cells and are non-pathogenic in humans often due to elevated sensitivity to innate antiviral signaling or dependence on oncogenic signaling pathways; and (ii) viruses that are genetically-manipulated for use. In some embodiments, the oncolytic virus can be a measles virus, a poliovirus, a poxvirus, a vaccinia virus, an adenovirus, an adeno associated virus, a herpes simplex virus, a vesicular stomatitis virus, a reovirus, a Newcastle disease virus, a senecavirus, a lentivirus, a mengovirus, or a myxoma virus. In certain embodiments, the oncolytic virus can be a poxvirus. In certain embodiments, the oncolytic virus can be a vaccinia virus.

The term “modified oncolytic virus” as used herein can refer to an oncolytic virus that comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of an exogenous protein or modified viral protein to the viral capsid. In general, oncolytic viruses may be modified (also known as “engineered”) in order to gain improved therapeutic effects against tumor cells. In some embodiments, the modified oncolytic virus can be a modified poxvirus. In some embodiments, the modified oncolytic virus can be a modified poxvirus. In some embodiments, the modified oncolytic virus can be a modified vaccinia virus.

The terms “systemic delivery,” and “systemic administration,” used interchangeably herein, in some cases can refer to a route of administration of medication, oncolytic virus or other substances into the circulatory system. The systemic administration may comprise intravenous administration, oral administration, intraperitoneal administration, parenteral administration, intranasal administration, sublingual administration, rectal administration, transdermal administration, intra-arterial administration, or any combinations thereof.

Soluble Programmed Cell Death Protein 1 (sPD-1)

Immune checkpoint inhibitors block checkpoint proteins from binding their receptor, thereby allowing the immune response to proceed. Use of a checkpoint inhibitor in cancer treatment circumvents a cancer cell's ability to avoid attack by T cells. Native Programmed cell death protein 1 (PD-1) comprises a transmembrane domain that anchors the protein to the surface of expressing cells. A variant of PD-1 truncated prior to the transmembrane domain provides for a soluble version of PD-1 (SPD-1). sPD-1 can act as a dominant negative decoy receptor to bind and sequester PD-L1, blocking the PD-1:PD-L1 signaling pathway. Blocking binding to PD-L1 allows for an increase in T cell activity and T cell-mediated killing (FIG. 1B).

Provided herein are compositions comprising nucleic acids encoding for PD-1 immune checkpoint inhibitors. In some embodiments, the PD-1 inhibitor prevents interaction of PD-1 on tumor cells with PD-L1 on T cells. In some embodiments, the PD-1 inhibitor comprises a modified PD-1. In some embodiments, the PD-1 is a murine PD-1. In some embodiments, the PD-1 is a human PD-1.

Native PD-1 is a membrane-bound 288 amino acid protein. Domains starting at the amino terminus include the extracellular domain, transmembrane domain, and cytoplasmic domain. In murine PD-1, the extracellular domain comprises 168 amino acids (SEQ ID NO: 2). In human PD-1, the extracellular domain comprises 170 amino acids (SEQ ID NO: 4). Exemplary sequences for inclusion in compositions described herein are listed in Table 1, SEQ ID NOs: 1-4.

TABLE 1
Amino Acid Sequences of PD-1

| | | UniProtKB | |
|---|---|---|---|
| SEQ ID | | accession | |
| NO: | Variant | ID | Amino Acid Sequence |
| 1 | murine | | MWVRQVPWSFTWAVLQLSWQSGWLLEVPNG |
| | native PD-1 | | PWRSLTFYPAWLTVSEGANATFTCSLSNWSED |
| | | | LMLNWNRLSPSNQTEKQAAFCNGLSQPVQDA |
| | | | RFQIIQLPNRHDFHMNILDTRRNDSGIYLCGAIS |
| | | | LHPKAKIEESPGAELVVTERILETSTRYPSPSPKP |
| | | | EGRFQGMVIGIMSALVGIPVLLLLAWALAVFCS |
| | | | TSMSEARGAGSKDDTLKEEPSAAPVPSVAYEE |
| | | | LDFQGREKTPELPTACVHTEYATIVFTEGLGAS |
| | | | AMGRRGSADGLQGPRPPRHEDGHCSWPL |
| 2 | murine sPD- | | MWVRQVPWSFTWAVLQLSWQSGWLLEVPNG |
| | 1 | | PWRSLTFYPAWLTVSEGANATFTCSLSNWSED |
| | | | LMLNWNRLSPSNQTEKQAAFCNGLSQPVQDA |
| | | | RFQIIQLPNRHDFHMNILDTRRNDSGIYLCGAIS |
| | | | LHPKAKIEESPGAELVVTERILETSTRYPSPSPKP |
| | | | EGRFQG |
| 3 | human | | MQIPQAPWPVVWAVLQLGWRPGWFLDSPDRP |
| | native PD-1 | | WNPPTFSPALLVVTEGDNATFTCSFSNTSESFV |
| | | | LNWYRMSPSNQTDKLAAFPEDRSQPGQDCRFR |
| | | | VTQLPNGRDFHMSVVRARRNDSGTYLCGAISL |
| | | | APKAQIKESLRAELRVTERRAEVPTAHPSPSPRP |
| | | | AGQFQTLVVGVVGGLLGSLVLLVWVLAVICSR |
| | | | AARGTIGARRTGQPLKEDPSAVPVFSVDYGEL |
| | | | DFQWREKTPEPPVPCVPEQTEYATIVFPSGMGT |
| | | | SSPARRGSADGPRSAQPLRPEDGHCSWPL |
| 4 | human sPD- | | MQIPQAPWPVVWAVLQLGWRPGWFLDSPDRP |
| | 1 | | WNPPTFSPALLVVTEGDNATFTCSFSNTSESFV |
| | | | LNWYRMSPSNQTDKLAAFPEDRSQPGQDCRFR |
| | | | VTQLPNGRDFHMSVVRARRNDSGTYLCGAISL |
| | | | APKAQIKESLRAELRVTERRAEVPTAHPSPSPRP |
| | | | AGQFQTLV |

Provided here are compositions comprising nucleic acids encoding for a murine sPD-1. In some embodiments, the nucleic acid sequence encodes for a peptide described by SEQ ID NO: 2. In some embodiments, the encoded murine sPD-1 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 2.

Provided here are compositions comprising nucleic acids encoding for a human sPD-1. In some embodiments, the nucleic acid sequence encodes for a peptide described by SEQ ID NO: 4. In some embodiments, the encoded human sPD-1 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 4.

In some embodiments, the human sPD1 further comprises one or more substitutions as compared to the wild-type sequence (SEQ ID NO: 3). In some embodiments, the one or more mutations provides for an increase in binding affinity between the soluble PD-1 and PD-L1. In some embodiments, the one or more mutations comprises E61V, M70I, Q75F, K78W, K78L, E84F, S87W, A129H, A132L, K135M, or any combination thereof. Substitution positions are based on positions as in SEQ ID NO: 3.

### IL-12

Cytokines generally control growth and activity of immune system cells. IL-12 induces differentiation of T cells to T-helper 1 (Th1) cells. Th1 cells aid in clearing pathogens from the system. Provided here are compositions comprising nucleic acids encoding for IL-12 or a functional variant thereof. In some embodiments, the nucleic acid encodes for a first polypeptide comprising an Interleukin 12 (IL-12) or a functional variant thereof. In some embodiments, the IL-12 comprises a heterodimer comprising a subunit beta (IL-12b) and subunit alpha (IL-12a). In some embodiments, the nucleic acid encodes a murine IL-12 (mIL-12) sequence as described by SEQ ID NO: 5. In some instances, the encoded mIL-12 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 5. In some embodiments, the nucleic acid encodes a human IL-12 (hIL-12) sequence as described by SEQ ID NO: 7. In some instances, the encoded hIL-12 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 7.

Provided here are compositions comprising nucleic acids encoding for a murine IL-12 subunit alpha (IL-12a) (UniProtKB accession ID 43431.1). In some embodiments, the nucleic acid sequence encodes for a peptide described by SEQ ID NO: 6. In some instances, the encoded IL-12a comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 6.

Provided here are compositions comprising nucleic acids encoding for a murine IL-12 subunit beta (IL-12b) (UniProtKB accession ID P43432.1). In some embodiments, the nucleic acid sequence encodes for a peptide described by SEQ ID NO: 7. In some instances, the encoded IL-12b comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 7.

Provided here are compositions comprising nucleic acids encoding for a human IL-12 subunit alpha (hIL-12a) (UniProtKB accession ID P060595). In some embodiments, the nucleic acid sequence encodes for a peptide described by SEQ ID NO: 9. In some instances, the encoded hIL-12a comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 9.

Provided here are compositions comprising nucleic acids encoding for a human IL-12 subunit beta (hIL-12b) (UniProtKB accession ID P29460). In some embodiments, the nucleic acid sequence encodes for a peptide described by SEQ ID NO: 10). In some instances, the encoded IL-12b comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 10.

Exemplary amino acid sequences for IL-12 regions for inclusion in compositions described here are listed in Table 2.

TABLE 2
IL-12 amino acid sequences.

| SEQ ID | | UniProtKB | |
|---|---|---|---|
| NO: | Description | accession ID | Amino Acid Sequence |
| 5 | Murine IL-12 | | MCPQKLTISWFAIVLLVSPLMAMWELEKDVYV |
| | (mIL-12) | | VEVDWTPDAPGETVNLTCDTPEEDDITWTSDQR |
| | | | HGVIGSGKTLTITVKEFLDAGQYTCHKGGETLS |
| | | | HSHLLLHKKENGIWSTEILKNFKNKTFLKCEAP |
| | | | NYSGRFTCSWLVQRNMDLKFNIKSSSSSPDSRA |
| | | | VTCGMASLSAEKVTLDQRDYEKYSVSCQEDVT |
| | | | CPTAEETLPIELALEARQQNKYENYSTSFFIRDII |
| | | | KPDPPKNLQMKPLKNSQVEVSWEYPDSWSTPH |
| | | | SYFSLKFFVRIQRKKEKMKETEEGCNQKGAFLV |
| | | | EKTSTEVQCKGGNVCVQAQDRYYNSSCSKWA |
| | | | CVPCRVRSGGGGSGGGGSGGGGSGGGGSASAT |
| | | | LALLNHLSLARVIPVSGPARCLSQSRNLLKTTDD |
| | | | MVKTAREKLKHYSCTAEDIDHEDITRDQTSTLK |
| | | | TCLPLELHKNESCLATRETSSTTRGSCLPPQKTS |
| | | | LMMTLCLGSIYEDLKMYQTEFQAINAALQNHN |
| | | | HQQIILDKGMLVAIDELMQSLNHNGETLRQKPP |
| | | | VGEADPYRVKMKLCILLHAFSTRVVTINRVMG |
| | | | YLSSA |
| 6 | Murine IL-12 | P43431.1 | MCQSRYLLFLATLALLNHLSLARVIPVSGPARC |
| | alpha | | LSQSRNLLKTTDDMVKTAREKLKHYSCTAEDID |
| | (mIL-12a) | | HEDITRDQTSTLKTCLPLELHKNESCLATRETSS |
| | | | TTRGSCLPPQKTSLMMTLCLGSIYEDLKMYQTE |
| | | | FQAINAALQNHNHQQIILDKGMLVAIDELMQSL |
| | | | NHNGETLRQKPPVGEADPYRVKMKLCILLHAFS |
| | | | TRVVTINRVMGYLSSA |
| 7 | Murine IL-12 | P43432.1 | MCPQKLTISWFAIVLLVSPLMAMWELEKDVYV |
| | beta | | VEVDWTPDAPGETVNLTCDTPEEDDITWTSDQR |
| | (mIL-12b) | | HGVIGSGKTLTITVKEFLDAGQYTCHKGGETLS |
| | | | HSHLLLHKKENGIWSTEILKNFKNKTFLKCEAP |
| | | | NYSGRFTCSWLVQRNMDLKFNIKSSSSSPDSRA |
| | | | VTCGMASLSAEKVTLDQRDYEKYSVSCQEDVT |
| | | | CPTAEETLPIELALEARQQNKYENYSTSFFIRDII |
| | | | KPDPPKNLQMKPLKNSQVEVSWEYPDSWSTPH |
| | | | SYFSLKFFVRIQRKKEKMKETEEGCNQKGAFLV |
| | | | EKTSTEVQCKGGNVCVQAQDRYYNSSCSKWA |
| | | | CVPCRVRS |
| 8 | Human IL-12 | | MCHQQLVISWFSLVFLASPLVAIWELKKDVYV |
| | (hIL-12) | | VELDWYPDAPGEMVVLTCDTPEEDGITWTLDQ |
| | | | SSEVLGSGKTLTIQVKEFGDAGQYTCHKGGEVL |
| | | | SHSLLLLHKKEDGIWSTDILKDQKEPKNKTFLR |
| | | | CEAKNYSGRFTCWWLTTISTDLTFSVKSSRGSS |
| | | | DPQGVTCGAATLSAERVRGDNKEYEYSVECQE |
| | | | DSACPAAEESLPIEVMVDAVHKLKYENYTSSFFI |
| | | | RDIIKPDPPKNLQLKPLKNSRQVEVSWEYPDTW |
| | | | STPHSYFSLTFCVQVQGKSKREKKDRVFTDKTS |
| | | | ATVICRKNASISVRAQDRYYSSSWSEWASVPCS |
| | | | GGGGSGGGGSGGGGSGGGGSASWPPGSASQPP |
| | | | PSPAAATGLHPAARPVSLQCRLSMCPARSLLLV |
| | | | ATLVLLDHLSLARNLPVATPDPGMFPCLHHSQN |
| | | | LLRAVSNMLQKARQTLEFYPCTSEEIDHEDITKD |
| | | | KTSTVEACLPLELTKNESCLNSRETSFITNGSCL |
| | | | ASRKTSFMMALCLSSIYEDLKMYQVEFKTMNA |
| | | | KLLMDPKRQIFLDQNMLAVIDELMQALNFNSET |
| | | | VPQKSSLEEPDFYKTKIKLCILLHAFRIRAVTIDR |
| | | | VMSYLNAS |
| 9 | Human IL-12 | P060595 | WPPGSASQPPPSPAAATGLHPAARPVSLQCRLS |
| | alpha | | MCPARSLLLVATLVLLDHLSLARNLPVATPDPG |
| | (hIL-12a) | | MFPCLHHSQNLLRAVSNMLQKARQTLEFYPCTS |
| | | | EEIDHEDITKDKTSTVEACLPLELTKNESCLNSR |
| | | | ETSFITNGSCLASRKTSFMMALCLSSIYEDLKMY |
| | | | QVEFKTMNAKLLMDPKRQIFLDQNMLAVIDEL |
| | | | MQALNFNSETVPQKSSLEEPDFYKTKIKLCILLH |
| | | | AFRIRAVTIDRVMSYLNAS |
| 10 | Human IL-12 | P29460 | MCHQQLVISWFSLVFLASPLVAIWELKKDVYV |
| | beta | | VELDWYPDAPGEMVVLTCDTPEEDGITWTLDQ |
| | (hIL-12b) | | SSEVLGSGKTLTIQVKEFGDAGQYTCHKGGEVL |
| | | | SHSLLLLHKKEDGIWSTDILKDQKEPKNKTFLR |
| | | | CEAKNYSGRFTCWWLTTISTDLTFSVKSSRGSS |
| | | | DPQGVTCGAATLSAERVRGDNKEYEYSVECQE |
| | | | DSACPAAEESLPIEVMVDAVHKLKYENYTSSFFI |
| | | | RDIIKPDPPKNLQLKPLKNSRQVEVSWEYPDTW |
| | | | STPHSYFSLTFCVQVQGKSKREKKDRVFTDKTS |
| | | | ATVICRKNASISVRAQDRYYSSSWSEWASVPCS |

### Linkers and Signaling Domains

Provided herein are compositions comprising a nucleic acid encoding for a linker. In some embodiments, the nucleic acid encoding for the linker is located between various encoded biologically functional units described herein. In some embodiments, the encoded linker is flexible or rigid. In further embodiments, the encoded linker is a cleavable linker. In further embodiments, the encoded cleavable linker comprises a disulfide bond. In further embodiments, the encoded cleavable linker comprises a protease sensitive domain. A non-limiting list of exemplary linkers encoded by nucleic acids comprised in compositions described herein is listed in Table 3. In some embodiments, a composition described herein comprises a nucleic acid encoding for a linker having a sequence as described by SEQ ID NO: 11. In some embodiments, a nucleic acid encodes for a linker comprising at least 70%, at least 75%, at least 80%, at least 85%, at least 90%, at least 95% sequence identity to SEQ ID NO: 11.

TABLE 3
Linkers.

| SEQ ID NO: | Linker type | Amino Acid Sequence |
|---|---|---|
| 11 | Flexible | GGGGSGGGGSGGGGSGGGGSAS |
| 12 | Flexible | (GGGGS)₁₋₃ |
| 13 | Flexible | (Gly)₆₋₈ |
| 14 | Rigid | (EAAAK)₁₋₃ |
| 15 | Rigid | A(EAAAK)₄ALEA(EAAAK)₄A |
| 16 | Rigid | PAPAP |
| 17 | Cleavable | VSQTSKLTR↓AETVFPDV |
| 18 | Cleavable | PLG↓LWA |
| 19 | Cleavable | RVL↓AEA |
| 20 | Cleavable | EDVVCC↓SMSY |
| 21 | Cleavable | GGIEGR↓GS |
| 22 | Cleavable | TRHRQPR↓GWE |
| 23 | Cleavable | AGNRVRR↓SVG |
| 24 | Cleavable | RRRRRRR↓R↓R |
| 25 | Cleavable | GFLG↓ |
| 26 | Cleavable | GGGGSSPLGLWAGGGGS |

Subscript in sequence denotes repeats.
Arrow in sequence indicates location of cleavage site.

### Combination Nucleic Acid Sequences

Provided herein are compositions comprising nucleic acids encoding for a combination of technical features. In some embodiments, a nucleic acid encodes for an IL-12 dimer (IL-12) or functional variant thereof and a soluble PD-1 (sPD-1) protein or functional variant thereof. In some embodiments, a nucleic acid encodes for a murine IL-12 dimer (IL-12) and a murine sPD-1 protein or functional variant thereof. In some embodiments, the murine IL-2 comprises a sequence of SEQ ID NO: 5. In some embodiments, the murine sPD-1 comprises a sequence of SEQ ID NO: 2. In some embodiments, a nucleic acid encodes for a human IL-12 dimer and a human sPD-1 protein or functional variant thereof. In some embodiments, human IL-12 comprises a sequence of SEQ ID NO: 8. In some embodiments, the human sPD-1 comprises a sequence as set for the in SEQ ID NO: 4.

In alternative embodiments, two nucleic acids are provided, wherein a first nucleic acid encodes for a first polypeptide comprising an IL-12 or a functional variant thereof, and a second nucleic acid encodes for a second polypeptide comprising as sPD-1 (SPD-1) protein or a functional variant thereof. In some embodiments, the IL-12 comprises a sequence of SEQ ID NO: 5 or SEQ ID NO: 8. In some embodiments, the sPD-1 protein comprises a sequence of SEQ ID NO: 2 or SEQ ID NO: 4.

### Chemokine Receptors

In some embodiments, provided herein is a modified oncolytic virus comprising an exogenous nucleic acid, also referred to herein as a transgene, that encodes for a chemokine receptor. In some cases, the exogenous nucleic acid is a therapeutic transgene. In some embodiments, provided herein is a modified oncolytic virus comprising an exogenous nucleic acid that encodes for a membrane associated protein that degrades hyaluronan, such as a hyaluronidase. In some embodiments, provided herein is a modified oncolytic virus comprising exogenous nucleic acids that encode for both a chemokine receptor and a hyaluronidase.

Chemokines are chemotactic cytokines that regulate the trafficking and positioning of cells by activating the seven-transmembrane spanning chemokine receptors. In some cases, chemokines are divided into four subfamilies based on the position of the first two N-terminal cysteine residues, including the CC, CXC, CX3C and XC subfamilies. Differential expression of chemokine receptors on leukocytes optionally results in selective recruitment of specific cell types under particular conditions, providing appropriate and efficient immune responses tailored to the infecting pathogen or foreign insult. Beyond their pivotal role in the coordinated migration of immune cells to the site of inflammation, in many cases, chemokines also play important roles in the development of lymphoid tissues, in the maturation of immune cells, and in the generation and delivery of adaptive immune responses.

Tumors are increasingly recognized as a complex microenvironment made up of many different cell types that cohabit and communicate with each other in a complicated signaling network. Chemokines are essential coordinators of cellular migration and cell-cell interactions and therefore have great impact on tumor development. In the tumor microenvironment, tumor-associated host cells and cancer cells release an array of different chemokines, resulting in the recruitment and activation of different cell types that mediate the balance between antitumor and pro-tumor responses. In addition to their primary role as chemoattractants, chemokines, in many cases, are also involved in other tumor-related processes, including tumor cell growth, angiogenesis and metastasis.

Tumor cells have been shown to acquire the ability to produce growth-promoting chemokines. For instance, melanoma has been found to express a number of chemokines, including CXCL1, CXCL2, CXCL3, CXCL8, CCL2 and CCL5, which have been implicated in tumor growth and progression. CCL2 level can be found increased in neuroblastoma cell lines and primary tumor cells isolated from human patients. Immunostaining studies also suggest an elevated expression level of CXCL12 in a variety of cancers, including breast cancer, carcinoid, cervical cancer, colorectal cancer, endometrial cancer, liver cancer, lung cancer, lymphoma, melanoma, ovarian cancer, pancreatic cancer, prostate cancer, stomach cancer.

Chemokine receptors are cytokine receptors found on the surface of certain cells that interact with chemokines. There have been 20 distinct chemokine receptors discovered in humans. Each has a 7-transmembrane structure and couples to G-protein for signal transduction within a cell, making them members of a large protein family of G protein-coupled receptors. Following interaction with their specific chemokine ligands, chemokine receptors trigger a flux in intracellular calcium (Ca²⁺) ions (calcium signaling). This causes cell responses, including the onset of a process known as chemotaxis that traffics the cell to a desired location within the organism. In general, the term “chemokine receptor” as used herein can refer to a membrane associated protein that selectively binds to a chemokine ligand and induces the chemotaxis toward the chemokine ligand.

It is to be understood the chemokine receptor as disclosed herein in some cases refers to not only the naturally occurring chemokine receptors identified in human bodies, but also include chemokine receptors from other sources, such as, but not limited to: (1) naturally occurring chemokine receptors identified in animals, like pigs, dogs, cows, sheep; and (2) non-naturally occurring chemokine receptors, like mutant proteins, chimeric receptors, design proteins with binding affinity to a certain type(s) of chemokines. In some examples, a fragment of a naturally occurring chemokine receptor is also considered a chemokine receptor, if the function of binding and responding to the corresponding chemokine and directing the chemotaxis of the cell is retained in the fragment. As provided herein, in some embodiments, the virus that comprises the exogenous nucleic acid encoding for the chemokine receptor forces a virus-infected cell to express the chemokine receptor as the virus hijacks the host cell's gene expression machinery.

In some cases, the modified oncolytic viruses comprise exogenous nucleic acid that encode for a cytokine receptor whose cognate cytokine is expressed in tumor microenvironments (e.g., IL15-R has a cognate cytokine IL15 expressed in a tumor microenvironment). In some cases, the modified oncolytic viruses encodes for a chemokine receptor(s) whose cognate chemokine(s) are likely to be expressed on tumors (e.g., CXCR4 has a cognate chemokine CXCL12 expressed on a tumor; CCR2 has a target CCL2 expressed on a tumor) and is delivered systemically as a naked virus. Subsequent to entry of the modified oncolytic viruses into the blood stream, by systemic delivery, the viruses infect lymphocytes, such as B-cells, and re-direct the infected B-cells to the tumor, resulting in significantly increased viral load in the tumor. In certain embodiments, the increased viral load in the tumor is achieved soon after the systemic delivery. Ability to deliver the modified oncolytic viruses disclosed herein, in a systemic manner, provides an advantage over traditional intratumoral delivery methods for oncolytic viruses. While intratumoral delivery is helpful in treating easily accessible tumors, in some instances, it is critical to treat inaccessible or metastatic cancer which is allegedly the predominant cause of death from the disease. In this context, it is ineffective to rely on oncolytic viruses delivered intratumorally, as it will need systemic dissemination after administration to the distant sites. However, this dissemination is often transient and ineffective, at least in part, due to the development of immune responses to the viral infection.

Chemokine receptors are divided into different families. Non-limiting examples of chemokine receptors, as described herein include CXC chemokine receptors, CC chemokine receptors, CX3C chemokine receptors and XC chemokine receptors that correspond to the 4 distinct subfamilies of chemokines they bind. Among the CXC chemokine receptors, CXCR1 and CXCR2 are closely related, while CXCR1 binds to CXCL8 and CXCL6, and CXCR2 binds to CXCL1 and CXCL7; CXCR3 binds to CXCL9, CXCL10, and CXCL11; CXCR4 binds to CXCL12 (or SDF-1); CXCR5 binds to CXCL13; CXCR6 binds to CXCL16. Among the CC chemokine receptors, CCR1's ligands include CCL4, CCL5, CCL6, CCL14, CCL15, CCL16, CCL23; CCR2's ligands include CCL2, CCL8, and CCL16; CCR3's ligands include CCL11, CCL26, CCL7, CCL13, CCL15, CCL24, CCL5, CCL28, and CCL18; CCR4's ligands include CCL3, CCL5, CCL17, and CCL22; CCR5's ligands include CCL3, CCL4, CCL5, CCL8, CCL11, CCL13, CCL14, and CCL16; CCR6's ligands include CCL20; CCR7's ligands include CCL19 and CCL21; CCR8's ligands include CCL1, CCL16; CCR9's ligands include CCL25; CCR10's ligand include CCL27, CCL28; CCR11's ligands include CCL19, CCL21, CCL25. CX3C chemokine receptor CX3CR1 has a ligand CXCL1. XC chemokine receptor XCR1 binds to both XCL1 and XCL2.

Non-limiting embodiments of the present disclosure provide a modified oncolytic virus that comprises an exogenous nucleic acid that encodes for a chemokine receptor. In some embodiments, the chemokine receptor is a CXC chemokine receptor, a CC chemokine receptor, a CX3C chemokine receptor, a XC chemokine receptor, or any combinations thereof. In some embodiments, the chemokine receptor is CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, XCR1, or any combinations thereof.

In certain embodiments, the modified oncolytic virus comprises an exogenous CXCR4-expressing nucleic acid. In certain embodiments, the modified oncolytic virus comprises an exogenous CCR2-expressing nucleic acid. Certain embodiments disclose a modified oncolytic virus comprising an exogenous nucleic acid that encodes for both CXCR4 and CCR2, and both chemokines are expressed from the same virus. Under certain circumstances, CXCL12 and/or CCL2 typically expressed in the tumor microenvironment attracts the CXCR4 and/or CCR2-expressing lymphocytes or other migrating cells that are infected by the modified oncolytic virus, thereby enhancing the tumor-targeted delivery of the modified oncolytic virus. Nucleic acid and amino acid sequences of selected chemokine receptors are listed in Table 4.

TABLE 4
Chemokine receptor sequences.

| SEQ ID | | |
|---|---|---|
| NO: | Description | Amino Acid Sequence |
| 27 | Murine CXCR3 | MYLEVSERQVLDASDFAFLLENSTSPYDYGENESDF |
| | | SDSPPCPQDFSLNFDRTFLPALYSLLFLLGLLGNGAV |
| | | AAVLLSQRTALSSTDTFLLHLAVADVLLVLTLPLW |
| | | AVDAAVQWVFGPGLCKVAGALFNINFYAGAFLLA |
| | | CISFDRYLSIVHATQIYRRDPRVRVALTCIVVWGLC |
| | | LLFALPDFIYLSANYDQRLNATHCQYNFPQVGRTAL |
| | | RVLQLVAGFLLPLLVMAYCYAHILAVLLVSRGQRR |
| | | FRAMRLVVVVVAAFAVCWTPYHLVVLVDILMDVG |
| | | VLARNCGRESHVDVAKSVTSGMGYMHCCLNPLLY |
| | | AFVGVKFREQMWMLFTRLGRSDQRGPQRQPSSSRR |
| | | ESSWSETTEASYLGL |
| 28 | Human CXCR3 isoform 1 | MVLEVSDHQVLNDAEVAALLENFSSSYDYGENESD |
| | | SCCTSPPCPQDFSLNFDRAFLPALYSLLFLLGLLGNG |
| | | AVAAVLLSRRTALSSTDTFLLHLAVADTLLVLTLPL |
| | | WAVDAAVQWVFGSGLCKVAGALFNINFYAGALLL |
| | | ACISFDRYLNIVHATQLYRRGPPARVTLTCLAVWGL |
| | | CLLFALPDFIFLSAHHDERLNATHCQYNFPQVGRTA |
| | | LRVLQLVAGFLLPLLVMAYCYAHILAVLLVSRGQR |
| | | RLRAMRLVVVVVVAFALCWTPYHLVVLVDILMDL |
| | | GALARNCGRESRVDVAKSVTSGLGYMHCCLNPLL |
| | | YAFVGVKFRERMWMLLLRLGCPNQRGLQRQPSSS |
| | | RRDSSWSETSEASYSGL |
| 29 | Human CCR2 var 467 | MLSTSRSRFIRNTNESGEEVTTFFDYDYGAPCHKFD |
| | | VKQIGAQLLPPLYSLVFIFGFVGNMLVVLILINCKKL |
| | | KCLTDIYLLNLAISDLLFLITLPLWAHSAANEWVFG |
| | | NAMCKLFTGLYHIGYFGGIFFIILLTIDRYLAIVHAVF |
| | | ALKARTVTFGVVTSVITWLVAVFASVPGIIFTKCQK |
| | | EDSVYVCGPYFPRGWNNFHTIMRNILGLVLPLLIMV |
| | | ICYSGILKTLLRCRNEKKRHRAVRVIFTIMIVYFLFW |
| | | TPYNIVILLNTFQEFFGLSNCESTSQLDQATQVTETL |
| | | GMTHCCINPIIYAFVGEKFRSLFHIALGCRIAPLQKP |
| | | VCGGPGVRPGKNVKVTTQGLLDGRGKGKSIGRAPE |
| | | ASLQDKEGA |
| 30 | CCR2 var 502 | MLSTSRSRFIRNTNESGEEVTTFFDYDYGAPCHKFD |
| | | VKQIGAQLLPPLYSLVFIFGFVGNMLVVLILINCKKL |
| | | KCLTDIYLLNLAISDLLFLITLPLWAHSAANEWVFG |
| | | NAMCKLFTGLYHIGYFGGIFFIILLTIDRYLAIVHAVF |
| | | ALKARTVTFGVVTSVITWLVAVFASVPGIIFTKCQK |
| | | EDSVYVCGPYFPRGWNNFHTIMRNILGLVLPLLIMV |
| | | ICYSGILKTLLRCRNEKKRHRAVRVIFTIMIVYFLFW |
| | | TPYNIVILLNTFQEFFGLSNCESTSQLDQATQVTETL |
| | | GMTHCCINPIIYAFVGEKFRSLFHIALGCRIAPLQKP |
| | | VCGGPGVRPGKNVKVTTQGLLDGRGKGKSIGRAPE |
| | | ASLQDKEGA |
| 31 | Human CXCR2 | MEDFNMESDSFEDFWKGEDLSNYSYSSTLPPFLLD |
| | | AAPCEPESLEINKYFWIIYALVFLLSLLGNSLVMLVI |
| | | LYSRVGRSVTDVYLLNLALADLLFALTLPIWAASK |
| | | VNGWIFGTFLCKWSLLKEVNFYSGILLLACISVDRY |
| | | LAIVHATRTLTQKRYLVKFICLSIWGLSLLLALPVL |
| | | LFRRTVYSSNVSPACYEDMGNNTANWRMLLRILP |
| | | QSFGFIVPLLIMLFCYGFTLRTLFKAHMGQKHRAM |
| | | RVIFAWLIFLLCWLPYNLVLLADTLMRTQVIQETC |
| | | ERRNHIDRALDATEILGILHSCLNPLIYAFIGQKFRH |
| | | GLLKILAIHGLISKDSLPKDSRPSFVGSSSGHTSTTL |
| 32 | Human CCR2 | MLSTSRSRFIRNTNESGEEVTTFFDYDYGAPCHKFD |
| | | VKQIGAQLLPPLYSLVFIFGFVGNMLVVLILINCKK |
| | | LKCLTDIYLLNLAISDLLFLITLPLWAHSAANEWVF |
| | | GNAMCKLFTGLYHIGYFGGIFFIILLTIDRYLAIVHA |
| | | VFALKARTVTFGVVTSVITWLVAVFASVPGIIFTKC |
| | | QKEDSVYVCGPYFPRGWNNFHTIMRNILGLVLPLL |
| | | IMVICYSGILKTLLRCRNEKKRHRAVRVIFTIMIVYF |
| | | LFWTPYNIVILLNTFQEFFGLSNCESTSQLDQATQV |
| | | TETLGMTHCCINPIIYAFVGEKFRSLFHIALGCRIAP |
| | | LQKPVCGGPGVRPGKNVKVTTQGLLDGRGKGKSI |
| | | GRAPEASLQDKEGA |
| 33 | Human CXCR4, Y157A | MEGISIYTSDNYTEEMGSGDYDSMKEPCFREENAN |
| | | FNKIFLPTIYSIIFLTGIVGNGLVILVMGYQKKLRSM |
| | | TDKYRLHLSVADLLFVITLPFWAVDAVANWYFGN |
| | | FLCKAVHVIYTVNLYSSVLILAFISLDRYLAIVHAT |
| | | NSQRPRKLLAEKVVAVGVWIPALLLTIPDFIFANVS |
| | | EADDRYICDRFYPNDLWVVVFQFQHIMVGLILPGI |
| | | VILSCYCIIISKLSHSKGHQKRKALKTTVILILAFFAC |
| | | WLPYYIGISIDSFILLEIIKQGCEFENTVHKWISITEA |
| | | LAFFHCCLNPILYAFLGAKFKTSAQHALTSVSRGSS |
| | | LKILSKGKRGGHSSVSTESESSSFHSS |
| 34 | Mouse CXCR4, Y159A | MEPISVSIYTSDNYSEEVGSGDYDSNKEPCFRDENV |
| | | HENRIFLPTIYFIIFLTGIVGNGLVILVMGYQKKLRS |
| | | MTDKYRLHLSVADLLFVITLPFWAVDAMADWYF |
| | | GKFLCKAVHIIYTVNLYSSVLILAFISLDRYLAIVHA |
| | | TNSQRPRKLLAEKAVAVGVWIPALLLTIPDFIFADV |
| | | SQGDISQGDDRYICDRLYPDSLWMVVFQFQHIMV |
| | | GLVLPGIVILSCYCIIISKLSHSKGHQKRKALKTTVIL |
| | | ILAFFACWLPYYVGISIDSFILLGVIKQGCDFESIVH |
| | | KWISITEALAFFHCCLNPILYAFLGAKFKSSAQHAL |
| | | NSMSRGSSLKILSKGKRGGHSSVSTESESSSFHSS |
| 35 | Human CXCR4, wildtype | MEGISIYTSDNYTEEMGSGDYDSMKEPCFREENAN |
| | | FNKIFLPTIYSIIFLTGIVGNGLVILVMGYQKKLRSM |
| | | TDKYRLHLSVADLLFVITLPFWAVDAVANWYFGN |
| | | FLCKAVHVIYTVNLYSSVLILAFISLDRYLAIVHAT |
| | | NSQRPRKLLAEKVVYVGVWIPALLLTIPDFIFANVS |
| | | EADDRYICDRFYPNDLWVVVFQFQHIMVGLILPGI |
| | | VILSCYCIIISKLSHSKGHQKRKALKTTVILILAFFAC |
| | | WLPYYIGISIDSFILLEIIKQGCEFENTVHKWISITEA |
| | | LAFFHCCLNPILYAFLGAKFKTSAQHALTSVSRGSS |
| | | LKILSKGKRGGHSSVSTESESSSFHSS |
| 36 | Human CXCR4, Y157A var | MEGISIYTSDNYTEEMGSGDYDSMKEPCFREENAN |
| | | FNKIFLPTIYSIIFLTGIVGNGLVILVMGYQKKLRSM |
| | | TDKYRLHLSVADLLFVITLPFWAVDAVANWYFGN |
| | | FLCKAVHVIYTVNLYSSVLILAFISLDRYLAIVHAT |
| | | NSQRPRKLLAEKVVAVGVWIPALLLTIPDFIFANVS |
| | | EADDRYICDRFYPNDLWVVVFQFQHIMVGLILPGI |
| | | VILSCYCIIISKLSHSKGHQKRKALKTTVILILAFFAC |
| | | WLPYYIGISIDSFILLEIIKQGCEFENTVHKWISITEA |
| | | LAFFHCCLNPILYAFLGAKFKTSAQHALTSVSRGSS |
| | | LKILSKGKRGGHSSVSTESESSSFHSS |
| 37 | Mouse CXCR4, Y159A | MEPISVSIYTSDNYSEEVGSGDYDSNKEPCFRDENV |
| | var. | HENRIFLPTIYFIIFLTGIVGNGLVILVMGYQKKLRS |
| | | MTDKYRLHLSVADLLFVITLPFWAVDAMADWYF |
| | | GKFLCKAVHIIYTVNLYSSVLILAFISLDRYLAIVHA |
| | | TNSQRPRKLLAEKAVAVGVWIPALLLTIPDFIFADV |
| | | SQGDISQGDDRYICDRLYPDSLWMVVFQFQHIMV |
| | | GLVLPGIVILSCYCIIISKLSHSKGHQKRKALKTTVIL |
| | | ILAFFACWLPYYVGISIDSFILLGVIKQGCDFESIVH |
| | | KWISITEALAFFHCCLNPILYAFLGAKFKSSAQHAL |
| | | NSMSRGSSLKILSKGKRGGHSSVSTESESSSFHSS |
| 38 | Human wild-type CXCR4 | MEGISIYTSDNYTEEMGSGDYDSMKEPCFREENAN |
| | | FNKIFLPTIYSIIFLTGIVGNGLVILVMGYQKKLRSM |
| | | TDKYRLHLSVADLLFVITLPFWAVDAVANWYFGN |
| | | FLCKAVHVIYTVNLYSSVLILAFISLDRYLAIVHAT |
| | | NSQRPRKLLAEKVVYVGVWIPALLLTIPDFIFANVS |
| | | EADDRYICDRFYPNDLWVVVFQFQHIMVGLILPGI |
| | | VILSCYCIIISKLSHSKGHQKRKALKTTVILILAFFAC |
| | | WLPYYIGISIDSFILLEIIKQGCEFENTVHKWISITEA |
| | | LAFFHCCLNPILYAFLGAKFKTSAQHALTSVSRGSS |
| | | LKILSKGKRGGHSSVSTESESSSFHSS |
| 39 | Human CCL5 | MKVSAAALAVILIATALCAPASASPYSSDTTPCCFA |
| | | YIARPLPRAHIKEYFYTSGKCSNPAWFVTRKNRQV |
| | | CANPEKKWVREYINSLEMS |
| 40 | Human CCR5 | MDYQVSSPIYDINYYTSEPCQKINVKQIAARLLPPL |
| | | YSLVFIFGFVGNMLVILILINCKRLKSMTDIYLLNLA |
| | | ISDLFFLLTVPFWAHYAAAQWDFGNTMCQLLTGL |
| | | YFIGFFSGIFFIILLTIDRYLAWHAVFALKARTVTFG |
| | | WTSVITWWAVFASLPGIIFTRSQKEGLHYTCSSHFP |
| | | YSQYQFWKNFQTLKIVILGLVLPLLVMVICYSGILK |
| | | TLLRCRNEKKRHRAVRLIFTIMIVYFLFWAPYNIVL |
| | | LLNTFQEFFGLNNCSSSNRLDQAMQVTETLGMTHC |
| | | CINPIIYAFVGEKFRNYLLVFFQKHIAKRFCKCCSIF |
| | | QQEAPERASSVYTRSTGEQEISVGL |

In compositions provided herein, an oncolytic virus gene may be mutated or replaced with nucleic acid encoding for a chemokine receptor as listed in Table 4. In some embodiments, the chemokine receptor is a murine CXCR3 described by SEQ ID NO: 27. In some embodiments, the encoded murine CXCR3 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 27. In some embodiments, the chemokine receptor is a human CXCR3 described by SEQ ID NO: 28. In some embodiments, the encoded human CXCR3 comprises at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or about 100% sequence identity to SEQ ID NO: 28.

### Promoters

Provided herein are compositions comprising nucleic acids, wherein the nucleic acid encodes for at least one promoter region. A promoter region, or promoter, or promoter element, or regulatory region, refers to a nucleic acid sequence to which proteins bind to initiate transcription. Promoters are typically located 5′, or upstream, to a DNA coding region which they control. In some embodiments, a nucleic acid described herein comprises one promoter. In some embodiments, the one promoter drives transcription of all polypeptides encoded on the nucleic acid. In some embodiments, a nucleic acid described herein comprises a separate promoter for each polypeptide encoded on the nucleic acid. In some embodiments, the nucleic acid comprises two promoters, each driving transcription of one of two polypeptides encoded on the nucleic acid.

Timing of expression can be modulated by the structure of the promotor regulating the gene expression. The number and affinity of transcription factor binding sites determines the relative timing of expression between different promoter regions. A promoter with more transcription factor binding sites and/or higher binding affinity can drive expression earlier than a promoter with fewer or lower affinity binding sites.

Application of the relative temporal expression of proteins, can be leveraged to express particular factors from modified viruses as described herein either earlier or later in the infection process. An early promoter has repeated transcription factor binding sites. A late promoter has fewer binding sites than an early promoter. In some embodiments, a receptor is expressed using an early promoter. Expression early in infection allows for expression and processing by the cell, before cellular processes are disrupted. In some embodiments, one or more cytokines are expressed using a late promoter.

Provided herein, in some embodiments, are promoters comprising P7.5, P28, P135, TK promoter, A52R promoter, 454 promoter, PB8, LEO, PF11, F7L, H5R, mH5, H1L, A1L, J3R, E4L, I1L, I3L, 14L, 15L, I7L, T7, I2L, FP4b, ATI, P11, PFL1, L4R, T7 promoter, 28 kDa promoter, a short synthetic promoter (SSP), or any functional variant or combination thereof. In some embodiments, the promoter is an early promoter. In some embodiments, the early promoter comprises A52R, PB8, mH5, I4L, LEO, PF11, I3L, P7.5, TK promoter, F7L, H5R, a short synthetic promoter (SSP) or any variation or combination thereof. In some embodiments, the promoter is a late promoter. In some embodiments, the late promoter comprises SSP, P7.5, P28, P135, TK promoter, F7L, H5R, H1L, A1L, J3R, E4L, I1L, I5L, I7L, T7, I2L, FP4b, ATI, P11, PFL1, L4R, 28 kDa promoter or any functional variant or combination thereof. Sequences of selected promoters are listed in Table 5.

TABLE 5
Promoter nucleic acid sequences

| SEQ ID | | |
|---|---|---|
| NO: | Description | Nucleic Acid Sequence |
| 41 | P28 promoter | AAGCTCACAAAAAAAACTTCTCTAAATAGATTCGAGC |
| 42 | 454 promoter | GGATCATAAAAATATAGTAGAATTTCATTTTGTTTTTTTCTATG |
| | | CTATAAATAGCTTCTCGAGC |
| 43 | P135 promoter | CCTGCAGGGCGTCCTAAAATTAAAACAAATGATATAA |
| | (VACWR135) | |
| 44 | Vaccinia P7.5 | TCACTAATTCCAAACCCACCCGCTTTTTATAGTAAGTTTTTCAC |
| | | CCATAAATAATAAATACAATAATTAATTTCTCGTAAAAGTAGA |
| | | AAATATATTCTAATTTATTGCACGGTAAGGAAGTAGATCATAA |
| 45 | Vaccinia virus | GTGATGCGGTTTTGGCAGTACATCAATGGGCGTGGATAGCGGT |
| | early promoter | TTGACTCACGGGGATTTCCAAGTCTCCACCCCATTGACGTCAA |
| | | TGGGAGTTTGTTTTGGCACCAAAATCAACGGGACTTTCCAAAA |
| | | TGTCGTAACAACTCCGCCCCATTGACGCAAATGGGCGGTAGG |
| | | CGTGTACGGTGGGAGGTCTATATAAGCAGAGCT |
| 46 | Human PGK | GGGGTTGGGGTTGCGCCTTTTCCAAGGCAGCCCTGGGTTTGCG |
| | promoter | CAGGGACGCGGCTGCTCTGGGCGTGGTTCCGGGAAACGCAGC |
| | | GGCGCCGACCCTGGGTCTCGCACATTCTTCACGTCCGTTCGCA |
| | | GCGTCACCCGGATCTTCGCCGCTACCCTTGTGGGCCCCCCGGC |
| | | GACGCTTCCTGCTCCGCCCCTAAGTCGGGAAGGTTCCTTGCGG |
| | | TTCGCGGCGTGCCGGACGTGACAAACGGAAGCCGCACGTCTC |
| | | ACTAGTACCCTCGCAGACGGACAGCGCCAGGGAGCAATGGCA |
| | | GCGCGCCGACCGCGATGGGCTGTGGCCAATAGCGGCTGCTCA |
| | | GCAGGGCGCGCCGAGAGCAGCGGCCGGGAAGGGGCGGTGCG |
| | | GGAGGCGGGGTGTGGGGCGGTAGTGTGGGCCCTGTTCCTGCC |
| | | CGCGCGGTGTTCCGCATTCTGCAAGCCTCCGGAGCGCACGTCG |
| | | GCAGTCGGCTCCCTCGTTGACCGAATCACCGACCTCTCTCCCC |
| | | AG |

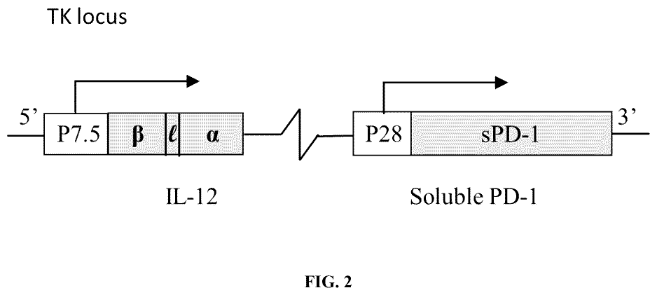

Compositions provided herein may comprise a P7.5 promoter and a P28 promoter. In some embodiments, the P7.5 promoter drives expression of a region encoding for an IL-12 polypeptide. In some embodiments, the P28 promoter drives transcription of a sPD-1. A schematic representation of promoter and transgene inserted at a TK locus is shown in FIG. 2. In some embodiments, the P7.5 promoter comprises a nucleic acid sequence of SEQ ID NO: 44. In some embodiments, the P28 promoter comprises a nucleic acid sequence of SEQ ID NO: 41.

Compositions provided herein may comprise a P135 promoter and a P7.5 promoter. In some embodiments, the P135 promoter drives expression of a region encoding for an IL-12 polypeptide. In some embodiments, the P7.5 promoter drives expression of a region encoding for sPD-1. In some embodiments, the P7.5 promoter comprises a nucleic acid sequence of SEQ ID NO: 44. In some embodiments, the P135 promoter comprises a sequence of SEQ ID NO: 43.

Provided herein are compositions comprising an exogenous nucleic acid. In some embodiments the exogenous nucleic acid comprises RNA. In some embodiments, the exogenous nucleic acid comprises DNA. In some embodiments, the DNA comprises, in 5′ to 3′ order, an IL-12 beta subunit, a linker, an IL-12 alpha subunit, and an sPD-1. In some embodiments, the DNA comprises, in 5′ to 3′ order, a P7.5 promoter (SEQ ID NO: 44), murine IL-12b (SEQ ID NO: 49), a flexible linker (SEQ ID NO: 50), murine IL-12a (SEQ ID NO: 51), a P28 promoter (SEQ ID NO: 41), and a murine sPD-1 (SEQ ID NO: 53). In some embodiments, the DNA comprises, in 5′ to 3′ order, a P7.5 promoter (SEQ ID NO: 44), murine IL-12 (SEQ ID NO: 48), a P28 promoter (SEQ ID NO: 41), and murine sPD-1 (SEQ ID NO: 53). In some embodiments, the DNA comprises, in 5′ to 3′ order, a P7.5 promoter (SEQ ID NO: 44), human IL-12b (SEQ ID NO: 61), a flexible linker (SEQ ID NO: 50), human IL-12a (SEQ ID NO: 62), a P28 promoter (SEQ ID NO: 41), and human sPD-1 (SEQ ID NO: 63). In some embodiments, the DNA comprises, in 5′ to 3′ order, a P7.5 promoter (SEQ ID NO: 44), human IL-12 (SEQ ID NO: 60), a P28 promoter (SEQ ID NO: 41), and human sPD-1 (SEQ ID NO: 60). In some embodiments, exogenous nucleic acids described herein are incorporated into a viral genome.

### Vectors

Provided herein are compositions comprising vectors. Generally, vectors are vehicles designed to carry a nucleic acid into a cell. In some embodiments, the vector comprises a plasmid, a phage, a virus, a cosmid, or an artificial chromosome. For the purposes of this application, a vector can also comprise transfection agents and methods, such as liposomes, nanoparticles, electroporation, microinjection, gene gun, impalefection, hydrostatic pressure, continuous infusion, sonication, or any combination thereof. Provided herein are compositions comprising a vector and one or more nucleic acids as described herein. In some embodiments, the vector comprises a virus. In some embodiments, the virus is a retrovirus, lentivirus, adenovirus, adeno-associated virus, or a herpes simplex virus. In some embodiments, the virus is an oncolytic virus. In some embodiments, the vector is a modified virus. In some embodiments, the virus comprises a mutation or deletion of one or more genes. In some embodiments, the virus comprises one or more exogenous nucleic acids as described herein.

### Oncolytic Viruses

Provided herein are compositions comprising an oncolytic virus wherein the oncolytic virus comprises a modified nucleic acid described herein. Oncolytic viruses, as used herein, kill cancer or tumor cells through mechanisms such as the direct lysis of said cells, by stimulating immune response towards said cells, apoptosis, expression of toxic proteins, autophagy and shutdown of protein synthesis, induction of anti-tumoral immunity, or any combinations thereof. In some embodiments, an oncolytic virus as described herein replicates within a cell. In some embodiments, an oncolytic virus as described herein replicates within a tumor cell, an immune cell, a somatic cell, a hemopoietic cell, or another type of cell. Exemplary oncolytic viruses for inclusion in a composition described herein include, without limitation, a poxvirus, a vaccinia virus, an adeno associated virus, an adenovirus, a reovirus, a lentivirus, a herpes simplex virus, a vesicular stomatitis virus, a mengovirus, a myxoma virus, Newcastle disease virus, a senecavirus, a retrovirus, measles virus, maraba virus, coxsackievirus, or polio virus. These oncolytic viruses have a proclivity to specifically target cancer cells, and upon virus replication cause significant cell death and tumor regression. In some embodiments, the oncolytic virus is a vaccinia virus. Exemplary vaccinia viruses include, without limitation, the following strains for modification by inclusion of an exogenous nucleic acid described herein: Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325). Furthermore, in some embodiments, the recombinant vaccinia viruses are modified versions of a wild type or attenuated vaccinia virus strain. Nonlimiting examples of vaccinia virus strains include a Western Reserve strain of vaccinia virus, a Copenhagen strain, a IHD strain, a Wyeth (NYCBOH) strain, a Tian Tan strain, a Lister strain, a USSR strain, an Ankara strain, an NYVAC strain, an Ankara (MVA) strain, a Paris strain, a Bern strain, a Temple of Heaven strain, a Dairen strain, an EM-63 strain, an Evans strain, a King strain, a Patwadangar strain, or a Tash Kent strain.

Oncolytic viruses are optionally recombinant or selected to have low toxicity and to accumulate in the target tissue. In some embodiments, the modifications in the viral backbone/viral genome are modifications that render the virus selectively replication competent. The base oncolytic virus strain modified as set forth herein optionally comprises one or more mutations or one or more deletions relative to its parent strain. In some embodiments, a modification includes mutation or complete or partial deletion in one or more of the following viral genes: A1, A2, VH1, A33, I7, A52R, TK, B15R, K7R, B14R, NIL, KIL, M2L, A49R, A46R, B8R, C12L, B18R, A52R, F3L, C4, or C16. In some embodiments, the viral backbone mutation is selected from the group consisting of: a complete or partial deletion of the A1 gene; a complete or partial deletion of the A2 gene; a complete or partial deletion of the VH1 gene; a complete or partial deletion of the A33 gene; a complete or partial deletion of 17 gene; a complete or partial deletion of the A52R gene; a complete or partial deletion of the TK gene; a complete or partial deletion of the B15R gene; a complete or partial deletion of the K7R gene; a complete or partial deletion of the B14R gene; a complete or partial deletion of the N1L gene; a complete or partial deletion of the K1L gene; a complete or partial deletion of the M2L gene; a complete or partial deletion of the A49R gene; a complete or partial deletion of the A46R gene; a complete or partial deletion of the B8R gene; a complete or partial deletion of the C12L gene; a complete or partial deletion of the B18R gene; a complete or partial deletion of the A52R gene; a complete or partial deletion of the F3L gene; a complete or partial deletion of the C4 gene; a complete or partial deletion of the C16 gene. As used herein, the reference to a viral gene is made by reference to the protein encoded by the gene (e.g., A33 gene means a gene that encodes for the A33 protein). In some embodiments, the viral backbone mutation, including any combinations of substitution, insertion, and deletion, result in a sequence with less than 100%, 99%, 98%, 97%, 96%, 95%, 94%, 93%, 92%, 91%, 90% or less sequence homology to the wild-type sequence of the viral gene or a viral protein encoded by the gene. In some embodiments, the viral backbone comprises 1, 2, 3, 4, 5, or more mutations in the amino acid sequence of the viral protein (e.g., a viral antigen). The disclosure provides in some embodiments, recombinant oncolytic viruses containing one more mutation(s) in the genome of the virus (virus back bone) such that the mutation increases the T-cell arm of the immune response. A mutation may be addition, deletion, or substitution of one or more nucleic acid(s) in the viral genome (wild type or attenuated native strains of oncolytic virus). In non-limiting examples, the mutation is complete or partial deletion of genes that are known to inhibit cytokines involved in the Th1 immune response. In some embodiments, the mutation is a deletion of nucleic acid encoding for B8R (interferon gamma (IFN-g) binding proteins). In some embodiments, the mutation is a deletion of nucleic acid encoding for C12L (interleukin-18 (IL-18) binding proteins). In some embodiments, the mutation is a complete or partial deletion of genes in innate immune signaling. In some embodiments, the mutation is a complete or partial deletion of nucleic acid encoding for B18R (type I interferon (IFN)-binding proteins). In some embodiments, the mutation is a complete or partial deletion of nucleic acid encoding for A52R (nuclear factor κB (NF-κB) inhibitor proteins). In some embodiments, the mutation is a complete or partial deletion of nucleic acid encoding for E3L (protein kinase (PKR) inhibitors), In some embodiments, the mutation is a complete or partial deletion of nucleic acid encoding for C4 or C16 (STING pathway inhibitors.

An oncolytic virus, as described herein, contains one or more additional insertions or partial insertions of exogenous nucleic acids that encode for one or more proteins. In some embodiments, the one or more proteins include a chemokine receptor or a functional variant thereof, soluble PD1 or a functional variant thereof, or interleukin-12 or a functional variant thereof. In some embodiments, the one or more proteins include sPD-1 or a functional variant thereof and interleukin-12 or a functional variant thereof. Exemplary chemokine receptors for inclusion include, without limitation, wild type and/or mutant type CXCR3, CXCR4, CCR2, or CCL2. An oncolytic virus of the current disclosure further contains one or more additional deletions or partial deletions of one or more genes from TK, A52R, B15R, K7R, A46R, NIL, E3L, KIL, M2L, C16, N2R, B8R, B18R, VH1 and a functional domain or fragment or variant thereof, or any combinations thereof. In some cases, the oncolytic virus provided herein contains a complete or partial deletion of at least one of: A52R or TK viral genes, and insertion of an exogenous nucleic acid encoding for one or more proteins (e.g., one or more immune modulator proteins). In some embodiments, the oncolytic virus further comprises a full or partial deletion of the B8R gene.

In some embodiments, the oncolytic virus is a modified oncolytic virus that has one or more modifications that results in a greater therapeutic effect against tumor cells, as compared to an otherwise identical virus that does not comprise the modifications. In some non-limiting examples, the greater therapeutic effect includes each or any combinations of: enhanced immune evasion of the virus, enhanced tumor-targeted systemic delivery of the virus, enhanced intratumoral and intertumoral spreading of the virus, and enhanced tumor-specific replication of the virus, or release of immune modulators and anti-tumor agents into the extracellular matrix. The modified oncolytic virus of this disclosure, in some instances, is utilized as a platform vector for systemic delivery.

Oncolytic viruses as described herein comprise exogenous nucleic acids described herein. In some embodiments, the oncolytic virus provided herein comprises a complete or partial deletion of the TK gene and an insertion of a region encoding for at least one of a soluble PD-1 and a cytokine, such as IL-12. Exemplary sequences for incorporation are described previously herein.

In some embodiments, the oncolytic virus provided herein contains a complete or partial deletion of the A52R gene and an insertion of a region encoding for a chemokine receptor. In some embodiments, the chemokine receptor comprises CXCR3. In some embodiments, region encoding for a chemokine receptor comprises a sequence selected from Table 4. In some embodiments, the promoter driving expression of the chemokine receptor is an early promoter, a late promoter, a strong early promoter, a weak early promoter, a strong late promoter, a weak late promoter, or any combination thereof. In some embodiments, an A52R promoter drives expression of the chemokine receptor. In some embodiments, an A52R promoter drives expression of the region encoding for CXCR3.

In some embodiments, provided herein is a modified oncolytic virus comprising a modification that enhances an immune response to a tumor. Typically, oncolytic viruses are either be (a) administered systemically, (b) inoculated topically over the tumor, or (c) injected directly into the tumor (“intratumoral delivery”).

In some embodiments, provided herein is a modified oncolytic virus comprising a modification that enhances intratumoral and intertumoral spreading of the virus. Enhanced spreading of the oncolytic virus within and between tumors is an effective manner to boost the therapeutic efficacy by increasing the number of the cancer cells that are infected by the virus. Provided herein, in some embodiments, is a modified oncolytic virus that comprises an exogenous nucleic acid. Provided herein, in some embodiments, is a modified oncolytic virus that comprises a modification in the genome of the virus. Provided herein, in some embodiments, is a modified oncolytic virus that comprises an exogenous nucleic acid as well as a modification in the genome of the virus.

In some embodiments, the oncolytic viruses include, but are not limited to, (i) viruses that naturally replicate preferentially in cancer cells and are non-pathogenic in humans often due to elevated sensitivity to innate antiviral signaling or dependence on oncogenic signaling pathways; and (ii) viruses that are genetically-manipulated for use.

In some embodiments, a modified oncolytic virus is employed. In general, such a virus comprises a modification to its constituent, such as, but not limited to, a modification in the native genome (“backbone”) of the virus like a mutation or a deletion of a viral gene, introduction of an exogenous nucleic acid, a chemical modification of a viral nucleic acid or a viral protein, and introduction of an exogenous protein or modified viral protein to the viral capsid.

In some embodiments, the modified oncolytic virus comprises a mutation or deletion of the TK gene and further comprises an exogenous nucleic acid that encodes for a sPD-1. In some embodiments, the modified oncolytic virus comprises a mutation or deletion of the TK gene, and further comprises an exogenous nucleic acid that encodes for a sPD-1, and an exogenous nucleic acid that encodes for a cytokine. In some embodiments, the cytokine comprises IL-12.

In some embodiments, the modified oncolytic virus comprises a mutation or deletion of the A52R gene and further comprises an exogenous nucleic acid that encodes for a CXCR3 receptor. In some embodiments, the modified oncolytic virus comprises a mutation or deletion of the A52R gene wherein the A52R promoter is maintained, and further comprises an exogenous nucleic acid that encodes for a CXCR3 receptor.

In some cases, the modified oncolytic virus comprises a full-length viral backbone gene or viral backbone protein described above, or truncated versions thereof, or functional domains thereof, or fragments thereof, or variants thereof. In various examples, the modified oncolytic virus comprises a mutation or deletion of one or more of viral backbone genes or viral backbone proteins, as described above. Mutations of the viral backbone genes and viral backbone proteins comprise insertion, deletion, substitution, or modifications of nucleotides in nucleic acid sequences and amino acids in protein sequences. Deletion comprises, in some examples, a complete or partial deletion of the viral backbone gene or protein.

In some embodiments, the modification of the oncolytic virus results in at least about 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 7.5×10⁵, 10⁶, 2.5×10⁶, 5×10⁶, 7.5×10⁶, 10⁷, 2.5×10⁷, 5×10⁷, 7.5×10⁷, 10⁸, 2.5×10⁸, 5×10⁸, 7.5×10⁸, 10⁹, 2.5×10⁹, 5×10⁹, 7.5×10⁹, 10¹⁰ or even more folds increase in the efficacy of tumor-targeted systemic delivery of the virus, as compared to an otherwise identical oncolytic virus that does not comprise the modification. In certain embodiments, the efficacy of tumor-targeted systemic delivery of the virus is measured by quantifying the viruses infecting the tumor cells, and optionally, in contrast with the viruses infecting non-tumor cells in the body. For instance, in some cases, the quantification of the virus is performed by staining the viral particles in tissue sections, or blood smear in the cases of leukemia, lymphoma, or myeloma. In some cases, such quantification is performed by reporter molecule(s) that is/are engineered to be expressed by the viruses, e.g., luciferase, and fluorescent proteins. In some cases, such quantification is performed by quantifying the viral genome in the tumor. Without being limited, it is also possible to measure the tumor-targeted systemic delivery of the virus by quantifying certain downstream effect(s) of viral infection in tumor cells, like cytokines in response to viral infection or lymphocyte accumulation. In some embodiments, the oncolytic virus comprises an exogenous nucleic acid that encodes for CXCR3, CXCR4, CCR2, or any combination thereof. In some embodiments, the presence of the exogenous nucleic acid results in about 5 to 10 folds increase in the efficacy of tumor-target systemic delivery of the virus, as compared to an otherwise identical oncolytic virus that does not comprise the exogenous nucleic acid.

In some embodiments, provided herein is a modified oncolytic virus comprising an exogenous nucleic acid that encodes for a soluble immune checkpoint inhibitor. Expression of the soluble immune checkpoint inhibitor by the modified oncolytic virus results in boosted immune responses against the infected tumor. Following infecting the tumor, the modified oncolytic virus replicates in the tumor cells and result in the expression of the soluble immune checkpoint inhibitor in the tumor environment. These soluble inhibitors function as decoy receptors, binding to the checkpoint ligand, blocking the immune response inhibition in the tumor. Consequently, the immunosuppressive microenvironment in the tumor is altered, leading to enhanced immunotherapeutic activity of the modified oncolytic virus, as compared to an otherwise identical virus that does not comprise the nucleic acid encoding for the chemokine receptor. In some embodiments, the increase in immunotherapeutic activity is at least about 1.1, 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 10⁶ or even higher folds. Without being limited, the increased immunotherapeutic activity is reflected by increased B cell accumulation in the tumor, increased T cell response to tumor-related immunogens, or both. B cell accumulation is measured, for example, by quantifying the B cells in the tumor, and T cell immuno-activity is measured by, for example, interferon-Y (interferon-gamma) secretion in ELISPOT assays.

In some embodiments, provided herein is a modified oncolytic virus that comprises an exogenous nucleic acid that encodes for a chemokine receptor, and the forced expression of chemokine receptor by the modified oncolytic virus results in boosted immune responses against the infected tumor. Following infecting the tumor, the modified oncolytic viruses replicate in the tumor cells and result in the expression of the chemokine receptors on the surface of the tumor cells. These membrane receptors function as decoy receptors, binding and sequestering the immunosuppressive chemokines within the tumor (e.g., CXCL12 and/or CCL2). Consequently, the immunosuppressive microenvironment in the tumor is altered, leading to enhanced immunotherapeutic activity of the modified oncolytic virus, as compared to an otherwise identical virus that does not comprise the nucleic acid encoding for the chemokine receptor. In some embodiments, the increase in immunotherapeutic activity is at least about 1.1, 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 10⁶ or even higher folds. Without being limited, the increased immunotherapeutic activity is reflected by increased B cell accumulation in the tumor, increased T cell response to tumor-related immunogens, or both. B cell accumulation is measured, for example, by quantifying the B cells in the tumor, and T cell immuno-activity is measured by, for example, interferon-Y (interferon-gamma) secretion in ELISPOT assays.

In some embodiments, provided herein is a modified oncolytic virus that comprises an exogenous nucleic acid that encodes for a chemokine receptor, and the forced expression of chemokine receptor by the modified oncolytic virus results in increased replication of the virus in tumor cells, as compared to an otherwise identical virus that does not comprise the nucleic acid encoding for the chemokine receptor. In some embodiments, the modified oncolytic virus comprises an exogenous CXCR3-expressing nucleic acid. In some embodiments, the modified oncolytic virus comprises an exogenous CCR2-expressing nucleic acid, which increases the tumor-specific replication of the virus. In some embodiments, the modified oncolytic virus comprises an exogenous CCR5-expressing nucleic acid, which increases the tumor-specific replication of the virus. In some embodiments, the increase in tumor-specific replication is at least about 1.1, 1.1, 1.2, 1.5, 1.8, 2, 2.2, 2.5, 2.8, 3, 3.2, 3.5, 3.8, 4, 4.2, 4.5, 4.8, 5, 5.2, 5.5, 5.8, 6, 6.2, 6.5, 6.8, 7, 7.2, 7.5, 7.8, 8, 8.2, 8.5, 8.8, 9, 9.2, 9.5, 9.8, 10, 12, 14, 15, 16, 18, 20, 25, 30, 35, 40, 45, 50, 55, 60, 65, 70, 75, 80, 85, 90, 95, 100, 150, 200, 250, 500, 800, 1000, 2500, 5000, 10⁴, 2.5×10⁴, 5×10⁴, 7.5×10⁴, 2.5×10⁵, 5×10⁵, 10⁶ or even higher folds. Exemplary methods for measuring the increase in viral delivery and spread in tumors include, but are not limited to, fluorescence or bioluminescence based imaging of expression of a reporter gene, quantitative PCR for detection of tumor concentrations of viral genomes or plaque determination of plaque forming units or immunohistochemistry of viral proteins.

### Conditions for Treatment

Provided herein are methods for treatment of cancer including administration of a composition described herein. In some embodiments, the method of treatment is for a hyperproliferative disease. In some embodiments, the hyperproliferative disease is a cancer. In some embodiments, the hyperproliferative disease comprises a tumor. Treatments comprising delivery of a modified oncolytic virus, such as an oncolytic vaccinia virus as described herein, is contemplated. In some embodiments, the cancer is melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, a myeloproliferative neoplasm, or sarcoma.

In some embodiments, compositions described herein are administered to cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer is optionally of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; or hairy cell leukemia. In some cases, solid cancers that are metastatic are treated using the modified oncolytic viruses of this disclosure, such as a modified oncolytic vaccinia virus that is advantageous for systemic delivery. In some cases, solid cancers that are inaccessible or difficult to access, such as for purpose of intratumoral delivery of therapeutic agents, are treated using the modified oncolytic viruses of this disclosure, such as a modified oncolytic vaccinia virus that is advantageous for systemic delivery. In some embodiments, compositions described herein are used to treat cancers that are associated with increased expression of free fatty acids.

This disclosure also contemplates methods for inhibiting or preventing local invasiveness or metastasis, or both, of any type of primary cancer. In exemplary embodiments, the primary cancer is melanoma, non-small cell lung, small-cell lung, lung, hepatocarcinoma, retinoblastoma, astrocytoma, glioblastoma, gum, tongue, leukemia, neuroblastoma, head, neck, breast, pancreatic, prostate, renal, bone, testicular, ovarian, mesothelioma, cervical, gastrointestinal, lymphoma, brain, colon, or bladder. In certain embodiments, the primary cancer is lung cancer. For example, the lung cancer is non-small cell lung carcinoma. Moreover, this disclosure optionally is used to prevent cancer or to treat pre-cancers or premalignant cells, including metaplasias, dysplasias, and hyperplasias. It can also be used to inhibit undesirable but benign cells, such as squamous metaplasia, dysplasia, benign prostate hyperplasia cells, hyperplastic lesions, and the like. In some embodiments, the progression to cancer or to a more severe form of cancer is halted, disrupted, or delayed by methods of this disclosure involving the modified oncolytic virus as discussed herein.

Provided herein are methods for treating a subject by administration of one or more modified oncolytic viruses, as disclosed herein. An “individual” or “subject,” as used interchangeably herein, refers to a human or a non-human subject. Non-limiting examples of non-human subjects include non-human primates, dogs, cats, mice, rats, guinea pigs, rabbits, pigs, fowl, horses, cows, goats, sheep, cetaceans, etc. In some embodiments, the subject is human.

Provided are methods for producing a toxic effect in a cancer cell comprising administering, to the cancer cell, a therapeutically effective amount of a modified virus, such as an oncolytic vaccinia virus, as described above, or a pharmaceutical composition containing the same. This disclosure further provides a method of inhibiting at least one of growth and proliferation of a second cancer cell comprising administering, to a first cancer cell, a modified oncolytic virus as described above such that the first cancer cell is infected with said virus. Thus, in some embodiments of the methods disclosed here, it is contemplated that not every cancer or tumor cell is infected upon administering a therapeutically effective amount of an oncolytic vaccinia virus, as described herein, or a pharmaceutical composition containing the same, and growth of non-infected cells is inhibited without direct infection.

In some examples, to induce oncolysis, kill cells, inhibit growth, inhibit metastases, decrease tumor size, and otherwise reverse or reduce the malignant phenotype of tumor cells, using the methods and compositions of the present disclosure, a cancer cell or a tumor is contacted with a therapeutically effective dose of an exemplary oncolytic vaccinia virus as described herein or a pharmaceutical composition containing the same. In certain embodiments, an effective amount of a modified oncolytic virus of the present disclosure, such as an oncolytic vaccinia virus as described herein or a pharmaceutical composition thereof, can include an amount sufficient to induce oncolysis, the disruption or lysis of a cancer cell or the inhibition or reduction in the growth or size of a cancer cell. Reducing the growth of a cancer cell is manifested, for example, by cell death or a slower replication rate or reduced growth rate of a tumor comprising the cell or a prolonged survival of a subject containing the cancer cell.

Provided herein are methods for treating a subject having a cancer or a tumor comprising administering, to the subject, an effective amount of a modified virus, as described above. An effective amount in such method includes an amount that reduces growth rate or spread of the cancer or that prolongs survival in the subject. This disclosure provides a method of reducing the growth of a tumor, which method comprises administering, to the tumor, an effective amount of a modified oncolytic virus as described above. In certain embodiments, an effective amount of a modified virus, or a pharmaceutical composition thereof, includes an amount sufficient to induce the slowing, inhibition or reduction in the growth or size of a tumor and includes the eradication of the tumor. Reducing the growth of a tumor is manifested, for example, by reduced growth rate or a prolonged survival of a subject containing the tumor. In certain embodiments, an effective amount of a modified virus, or a pharmaceutical composition thereof, includes an amount sufficient to activate an anti-tumor response. In some embodiments, activating an anti-tumor response includes activating T-cells. In certain embodiments, an effective amount of a modified virus, or a pharmaceutical composition thereof, includes an amount sufficient to reduce incidence of tumor growth. In some embodiments, reducing incidence of tumor growth includes suppression of metastasis, prevention of primary tumor growth, suppression of existing tumor growth, or any combination thereof.

Provided herein are methods for determining the infectivity or anti-tumor activity, or amount of tumor specific viral replication of an oncolytic vaccinia virus as described herein, which method comprises; (i) administering to a subject a therapeutically effective amount of an oncolytic vaccinia virus or a pharmaceutical composition according to the present disclosure, which further expresses a luciferase reporter gene, alone or in combination with a further therapy; (ii) collecting a first biological sample from the subject immediately after administering the virus and determining the level of the luciferase reporter in the first biological sample (iii) collecting a second biological sample from the subject following the administration in step (ii) and (iii) detecting the level of the luciferase reporter in the second biological sample, wherein the oncolytic vaccinia virus is determined to be infective, demonstrate anti-tumor activity, exhibit tumor specific viral replication if the level of luciferase is higher in step (iii) than in step (ii). The second biological sample is collected about 30 mins, about 1 hour, about 2 hours, about 3 hours, about 4 hours, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months after the administration in step (i). In some embodiments, the method of mentioned above further comprises, detecting in steps (i) and (iii), the level of one or more assaying cytokine levels, e.g., IL-2, IL-7, IL-8, IL-10, IFN-γ, GM-CSF, TNF-α, IL-6, IL-4, IL-5, and IL-13, in plasma samples collected from a subject after administering to said subject a therapeutically effective amount of a modified oncolytic virus of the present disclosure, such as an oncolytic vaccinia virus as described herein or a pharmaceutical composition comprising the same. In some embodiments of this disclosure, the increase in luciferase bioluminescence between steps (ii) and (iv) mentioned above is higher for a modified oncolytic virus as described herein, compared to that in an otherwise identical virus that does not comprise the modifications in the modified oncolytic virus. Other exemplary techniques for detecting and monitoring viral load after administration of the modified oncolytic viruses include real-time quantitative PCR.

Provided herein are methods for monitoring the pharmacokinetics following administration of a therapeutically effective amount of modified oncolytic viruses according to the present disclosure, such as oncolytic vaccinia virus or a pharmaceutical composition containing the vaccinia virus, as described herein. An exemplary method for monitoring the pharmacokinetics comprises the following steps: (i) administering to the subject a therapeutically effective amount of an oncolytic vaccinia virus or a pharmaceutical composition comprising the same, alone or in combination with a further therapy; (ii) collecting biological samples from the subject at one or more time points selected from about 15 minutes, about 30 minutes, about 45 mins, about 60 mins, about 75 mins, about 90 mins, about 120 mins, about 180 mins, and about 240 mins, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months after the administration in step (i); and (iii) detecting the quantity of the viral genome (or a reporter gene inserted within the viral genome, such as luciferase) in the biological samples collected at the above mentioned time points. In some instances, viral genome copies/mL is highest in the sample collected at the 15 mins time point and further the sample collected at the 240 mins time point does not contain a detectable quantity of the viral genome. Therefore, in some instances, a viral peak is observed at about 15 mins following administration and majority of the viruses is cleared from the subject's system after about 240 mins (or 4 hours). In some instances, a first viral peak is observed after about 15 mins following administration and a second viral peak is observed in the biological samples collected in the subsequent time points, e.g., at about 30 mins, about 45 mins, about 60 mins, or about 90 mins. The biological sample is, in exemplar embodiments, blood, and the quantity of viral genome/mL is determined by quantitative PCR or other appropriate techniques. In some examples, a first viral peak is observed after about 15 mins following administration and a second viral peak is observed after about 30 mins, about 1 hour, about 2 hours, about 3 hours, about 4 hours, about 5 hours, about 6 hours, about 7 hours, about 8 hours, about 9 hours, about 10 hours, about 12 hours, about 15 hours, about 24 hours, about 36 hours, about 48 hours, about 72 hours, about 4 days, about 5 days, about 6 days, about 7 days, about 8 days, about 9 days, about 10 days, about 11 days, about 12 days, about 13 days, about 14 days, about 15 days, about 1 month, to about 2 months following administration of a modified oncolytic virus of the present disclosure, such as an oncolytic vaccinia virus as described herein.

In some instances, tumor-selective replication of a modified virus, such as an oncolytic vaccinia virus is measured through use of a reporter gene, such as a luciferase gene. In some embodiments, the luciferase gene is inserted into the genome of a virus, and a tumor cell is infected with the virus. Bioluminescence in infected tumor cells is measured to monitor tumor-selective replication. Some examples show an increase in luciferase reporter bioluminescence in a modified oncolytic virus of this disclosure, compared to that in an otherwise identical oncolytic vaccinia virus that does not contain the modifications in the modified oncolytic virus.

### Dosage

In some embodiments, the amount of a modified oncolytic virus described herein administered to a subject is between about 10³ and 10¹² infectious viral particles or plaque forming units (PFU), or between about 10⁵ and 10¹⁰ PFU, or between about 10⁵ and 10⁸ PFU, or between about 10⁸ and 10¹⁰ PFU. In some embodiments, the amount of a modified oncolytic virus of this disclosure administered to a subject is between about 10³ and 10¹² viral particles or plaque forming units (PFU), or between about 10⁵ and 10¹⁰ PFU, or between about 10⁵ and 10⁸ PFU, or between about 10⁸ and 10¹⁰ PFU. In some embodiments, a modified oncolytic virus of this disclosure is administered at a dose that comprises about 10³ PFU/dose to about 10⁴ PFU/dose, about 10⁴ PFU/dose to about 10⁵ PFU/dose, about 10⁵ PFU/dose to about 10⁶ PFU/dose, about 10⁷ PFU/dose to about 10⁸ PFU/dose, about 10⁹ PFU/dose to about 10¹⁰ PFU/dose, about 10¹⁰ PFU/dose to about 10¹¹ PFU/dose, about 10¹¹ PFU/dose to about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose. In some embodiments, a modified oncolytic virus of this disclosure is administered at a dose that comprises about 2×10³ PFU/dose, 3×10³ PFU/dose, 4×10³ PFU/dose, 5×10³ PFU/dose, 6×10³ PFU/dose, 7×10³ PFU/dose, 8×10³ PFU/dose, 9×10³ PFU/dose, about 10⁴ PFU/dose, about 2×10⁴ PFU/dose, about 3×10⁴ PFU/dose, about 4×10⁴ PFU/dose, about 5×10⁴ PFU/dose, about 6×10⁴ PFU/dose, about 7×10⁴ PFU/dose, about 8×10⁴ PFU/dose, about 9×10⁴ PFU/dose, about 10⁵ PFU/dose, 2×10⁵ PFU/dose, 3×10⁵ PFU/dose, 4×10⁵ PFU/dose, 5×10⁵ PFU/dose, 6×10⁵ PFU/dose, 7×10⁵ PFU/dose, 8×10⁵ PFU/dose, 9×10⁵ PFU/dose, about 10⁶ PFU/dose, about 2×10⁶ PFU/dose, about 3×10⁶ PFU/dose, about 4×10⁶ PFU/dose, about 5×10⁶ PFU/dose, about 6×10⁶ PFU/dose, about 7×10⁶ PFU/dose, about 8×10⁶ PFU/dose, about 9×10⁶ PFU/dose, about 10⁷ PFU/dose, about 2×10⁷ PFU/dose, about 3×10⁷ PFU/dose, about 4×10⁷ PFU/dose, about 5×10⁷ PFU/dose, about 6×10⁷ PFU/dose, about 7×10⁷ PFU/dose, about 8×10⁷ PFU/dose, about 9×10⁷ PFU/dose, about 10⁸ PFU/dose, about 2×10⁸ PFU/dose, about 3×10⁸ PFU/dose, about 4×10⁸ PFU/dose, about 5×10⁸ PFU/dose, about 6×10⁸ PFU/dose, about 7×10⁸ PFU/dose, about 8×10⁸ PFU/dose, about 9×10⁸ PFU/dose, about 10⁹ PFU/dose, about 2×10⁹ PFU/dose, about 3×10⁹ PFU/dose, about 4×10⁹ PFU/dose, about 5×10¹⁰ PFU/dose, about 6×10⁹ PFU/dose, about 7×10⁹ PFU/dose, about 8×10⁹ PFU/dose, about 9×10⁹ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹⁰ PFU/dose, about 2×10¹⁰ PFU/dose, about 3×10¹⁰ PFU/dose, about 4×10¹⁰ PFU/dose, about 5×10¹⁰ PFU/dose, about 6×10¹⁰ PFU/dose, about 7×10¹⁰ PFU/dose, about 8×10¹⁰ PFU/dose, about 9×10¹⁰ PFU/dose, about 10¹¹ PFU/dose, about 2×10¹¹ PFU/dose, about 3×10¹¹ PFU/dose, about 4×10¹¹ PFU/dose, about 5×10¹¹ PFU/dose, about 6×10¹¹ PFU/dose, about 7×10¹¹ PFU/dose, about 8×10¹¹ PFU/dose, about 9×10¹¹ PFU/dose, or about 10¹² PFU/dose, about 10¹² PFU/dose to about 10¹³ PFU/dose, about 10¹³ PFU/dose to about 10¹⁴ PFU/dose, or about 10¹⁴ PFU/dose to about 10¹⁵ PFU/dose. In some embodiments, a modified oncolytic virus of this disclosure is administered at a dose that comprises 5×10⁹ PFU/dose. In some embodiments, a modified oncolytic virus of this disclosure is administered at a dose that comprises up to 5×10⁹ PFU/dose.

In some embodiments, a modified oncolytic virus of this disclosure is administered at a dose that comprises about 10³ viral particles/dose to about 10⁴ viral particles/dose, about 10⁴ viral particles/dose to about 10⁵ viral particles/dose, about 10⁵ viral particles/dose to about 10⁶ viral particles/dose, about 10⁷ viral particles/dose to about 10⁸ viral particles/dose, about 10⁹ viral particles/dose to about 10¹⁰ viral particles/dose, about 10¹⁰ viral particles/dose to about 10¹¹ viral particles/dose, about 10¹¹ viral particles/dose to about 10¹² viral particles/dose, about 10¹² viral particles/dose to about 10¹³ viral particles/dose, about 10¹³ viral particles/dose to about 10¹⁴ viral particles/dose, or about 10¹⁴ viral particles/dose to about 10¹⁵ viral particles/dose.

In some embodiments, a modified oncolytic virus of this disclosure is administered at a dose that comprises about 10³ PFU/kg to about 10⁴ PFU/kg, about 10⁴ PFU/kg to about 10⁵ PFU/kg, about 10⁵ PFU/kg to about 10⁶ PFU/kg, about 10⁷ PFU/kg to about 10⁸ PFU/kg, about 10⁹ PFU/kg to about 10¹⁰ PFU/kg, about 10¹⁰ PFU/kg to about 10¹¹ PFU/kg, about 10¹¹ PFU/kg to about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, a modified oncolytic virus of this disclosure is administered at a dose that comprises about 2×10³ PFU/kg, 3×10³ PFU/kg, 4×10³ PFU/kg, 5×10³ PFU/kg, 6×10³ PFU/kg, 7×10³ PFU/kg, 8×10³ PFU/kg, 9×10³ PFU/kg, about 10⁴ PFU/kg, about 2×10⁴ PFU/kg, about 3×10⁴ PFU/kg, about 4×10⁴ PFU/kg, about 5×10⁴ PFU/kg, about 6×10⁴ PFU/kg, about 7×10⁴ PFU/kg, about 8×10⁴ PFU/kg, about 9×10⁴ PFU/kg, about 10⁵ PFU/kg, 2×10⁵ PFU/kg, 3×10⁵ PFU/kg, 4×10⁵ PFU/kg, 5×10⁵ PFU/kg, 6×10⁵ PFU/kg, 7×10⁵ PFU/kg, 8×10⁵ PFU/kg, 9×10⁵ PFU/kg, about 10⁶ PFU/kg, about 2×10⁶ PFU/kg, about 3×10⁶ PFU/kg, about 4×10⁶ PFU/kg, about 5×10⁶ PFU/kg, about 6×10⁶ PFU/kg, about 7×10⁶ PFU/kg, about 8×10⁶ PFU/kg, about 9×10⁶ PFU/kg, about 10⁷ PFU/kg, about 2×10⁷ PFU/kg, about 3×10⁷ PFU/kg, about 4×10⁷ PFU/kg, about 5×10⁷ PFU/kg, about 6×10⁷ PFU/kg, about 7×10⁷ PFU/kg, about 8×10⁷ PFU/kg, about 9×10⁷ PFU/kg, about 10⁸ PFU/kg, about 2×10⁸ PFU/kg, about 3×10⁸ PFU/kg, about 4×10⁸ PFU/kg, about 5×10⁸ PFU/kg, about 6×10⁸ PFU/kg, about 7×10⁸ PFU/kg, about 8×10⁸ PFU/kg, about 9×10⁸ PFU/kg, about 10⁹ PFU/kg, about 2×10⁹ PFU/kg, about 3×10⁹ PFU/kg, about 4×10⁹ PFU/kg, about 5×10⁹ PFU/kg, about 6×10⁹ PFU/kg, about 7×10⁹ PFU/kg, about 8×10⁹ PFU/kg, about 9×10⁹ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹⁰ PFU/kg, about 2×10¹⁰ PFU/kg, about 3×10¹⁰ PFU/kg, about 4×10¹⁰ PFU/kg, about 5×10¹⁰ PFU/kg, about 6×10¹⁰ PFU/kg, about 7×10¹⁰ PFU/kg, about 8×10¹⁰ PFU/kg, about 9×10¹⁰ PFU/kg, about 10¹¹ PFU/kg, about 2×10¹¹ PFU/kg, about 3×10¹¹ PFU/kg, about 4×10¹¹ PFU/kg, about 5×10¹¹ PFU/kg, about 6×10¹¹ PFU/kg, about 7×10¹¹ PFU/kg, about 8×10¹¹ PFU/kg, about 9×10¹¹ PFU/kg, or about 10¹² PFU/kg, about 10¹² PFU/kg to about 10¹³ PFU/kg, about 10¹³ PFU/kg to about 10¹⁴ PFU/kg, or about 10¹⁴ PFU/kg to about 10¹⁵ PFU/kg. In some embodiments, a modified oncolytic virus of this disclosure is administered at a dose that comprises 5×10⁹ PFU/kg. In some embodiments, a modified oncolytic virus of this disclosure is administered at a dose that comprises up to 5×10⁹ PFU/kg.

In some embodiments, a modified oncolytic virus of this disclosure is administered at a dose that comprises about 10³ viral particles/kg to about 10⁴ viral particles/kg, about 10⁴ viral particles/kg to about 10⁵ viral particles/kg, about 10⁵ viral particles/kg to about 10⁶ viral particles/kg, about 10⁷ viral particles/kg to about 10⁸ viral particles/kg, about 10⁹ viral particles/kg to about 10¹⁰ viral particles/kg, about 10¹⁰ viral particles/kg to about 10¹¹ viral particles/kg, about 10¹¹ viral particles/kg to about 10¹² viral particles/kg, about 10¹² viral particles/kg to about 10¹³ viral particles/kg, about 10¹³ viral particles/kg to about 10¹⁴ viral particles/kg, or about 10¹⁴ viral particles/kg to about 10¹⁵ viral particles/kg.

A liquid dosage form of an oncolytic vaccinia virus as described herein comprises, in certain embodiments, a viral dose of about 10³ PFU/mL to about 10⁴ PFU/mL, about 10⁴ PFU/mL to about 10⁵ PFU/mL, about 10⁵ PFU/mL to about 10⁶ PFU/mL, about 10⁷ PFU/mL to about 10⁸ PFU/mL, about 10⁹ PFU/mL to about 10¹⁰ PFU/mL, about 10¹⁰ PFU/mL to about 10¹¹ PFU/mL, about 10¹¹ PFU/mL to about 10¹² PFU/mL, about 10¹² PFU/mL to about 10¹³ PFU/mL, about 10¹³ PFU/mL to about 10¹⁴ PFU/mL, or about 10¹⁴ PFU/mL to about 10¹⁵ PFU/mL. In some embodiments, a modified oncolytic virus of this disclosure is administered at a dose that comprises about 2×10³ PFU/mL, 3×10³ PFU/mL, 4×10³ PFU/mL, 5×10³ PFU/mL, 6×10³ PFU/mL, 7×10³ PFU/mL, 8×10³ PFU/mL, 9×10³ PFU/mL, about 10⁴ PFU/mL, about 2×10⁴ PFU/mL, about 3×10⁴ PFU/mL, about 4×10⁴ PFU/mL, about 5×10⁴ PFU/mL, about 6×10⁴ PFU/mL, about 7×10⁴ PFU/mL, about 8×10⁴ PFU/mL, about 9×10⁴ PFU/mL, about 10⁵ PFU/mL, 2×10⁵ PFU/mL, 3×10⁵ PFU/mL, 4×10⁵ PFU/mL, 5×10⁵ PFU/mL, 6×10⁵ PFU/mL, 7×10⁵ PFU/mL, 8×10⁵ PFU/mL, 9×10⁵ PFU/mL, about 10⁶ PFU/mL, about 2×10⁶ PFU/mL, about 3×10⁶ PFU/mL, about 4×10⁶ PFU/mL, about 5×10⁶ PFU/mL, about 6×10⁶ PFU/mL, about 7×10⁶ PFU/mL, about 8×10⁶ PFU/mL, about 9×10⁶ PFU/mL, about 10⁷ PFU/mL, about 2×10⁷ PFU/mL, about 3×10⁷ PFU/mL, about 4×10⁷ PFU/mL, about 5×10⁷ PFU/mL, about 6×10⁷ PFU/mL, about 7×10⁷ PFU/mL, about 8×10⁷ PFU/mL, about 9×10⁷ PFU/mL, about 10⁸ PFU/mL, about 2×10⁸ PFU/mL, about 3×10⁸ PFU/mL, about 4×10⁸ PFU/mL, about 5×10⁸ PFU/mL, about 6×10⁸ PFU/mL, about 7×10⁸ PFU/mL, about 8×10⁸ PFU/mL, about 9×10⁸ PFU/mL, about 10⁹ PFU/mL, about 2×10⁹ PFU/mL, about 3×10⁹ PFU/mL, about 4×10⁹ PFU/mL, about 5×10⁹ PFU/mL, about 6×10⁹ PFU/mL, about 7×10⁹ PFU/mL, about 8×10⁹ PFU/mL, about 9×10⁹ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹⁰ PFU/mL, about 2×10¹⁰ PFU/mL, about 3×10¹⁰ PFU/mL, about 4×10¹⁰ PFU/mL, about 5×10¹⁰ PFU/mL, about 6×10¹⁰ PFU/mL, about 7×10¹⁰ PFU/mL, about 8×10¹⁰ PFU/mL, about 9×10¹⁰ PFU/mL, about 10¹¹ PFU/mL, about 2×10¹¹ PFU/mL, about 3×10¹¹ PFU/mL, about 4×10¹¹ PFU/mL, about 5×10¹¹ PFU/mL, about 6×10¹¹ PFU/mL, about 7×10¹¹ PFU/mL, about 8×10¹¹ PFU/mL, about 9×10¹¹ PFU/mL, or about 10¹² PFU/mL, about 10¹² PFU/mL to about 10¹³ PFU/mL, about 10¹³ PFU/mL to about 10¹⁴ PFU/mL, or about 10¹⁴ PFU/mL to about 10¹⁵ PFU/mL. In some embodiments, a modified oncolytic virus of this disclosure is administered at a dose that comprises 5×10⁹ PFU/mL. In some embodiments, a modified oncolytic virus of this disclosure is administered at a dose that comprises up to 5×10⁹ PFU/mL.

In some instances, where the modified oncolytic virus is administered by an injection, the dosage comprises about 10³ viral particles per injection, 10⁴ viral particles per injection, 10⁵ viral particles per injection, 10⁶ viral particles per injection, 10⁷ viral particles per injection, 10⁸ viral particles per injection, 10⁹ viral particles per injection, 10¹⁰ viral particles per injection, 10¹¹ viral particles per injection, 10¹² viral particles per injection, 2×10¹² viral particles per injection, 10¹³ viral particles per injection, 10¹⁴ viral particles per injection, or 10¹⁵ viral particles per injection. In further instances, where the modified oncolytic virus is administered by an injection, the dosage comprises about 10³ infectious viral particles per injection, 10⁴ infectious viral particles per injection, 10⁵ infectious viral particles per injection, 10⁶ infectious viral particles per injection, 10⁷ infectious viral particles per injection, 10⁸ infectious viral particles per injection, 10⁹ infectious viral particles per injection, 10¹⁰ infectious viral particles per injection, 10¹¹ infectious viral particles per injection, 10¹² infectious viral particles per injection, 2×10¹² infectious viral particles per injection, 10¹³ infectious viral particles per injection, 10¹⁴ infectious viral particles per injection, or 10¹⁵ infectious viral particles per injection. In certain embodiments, the virus is administered in an amount sufficient to induce oncolysis in at least about 20% of cells in a tumor, in at least about 30% of cells in a tumor, in at least about 40% of cells in a tumor, in at least about 50% of cells in a tumor, in at least about 60% of cells in a tumor, in at least about 70% of cells in a tumor, in at least about 80% of cells in a tumor, or in at least about 90% of cells in a tumor. In certain embodiments, a single dose of virus refers to the amount administered to a subject or a tumor over a 1, 2, 5, 10, 15, 20 or 24 hour period. In certain embodiments, the dose is spread over time or by separate injection. In certain embodiments, multiple doses (e.g., 2, 3, 4, 5, 6 or more doses) of the vaccinia virus is administered to the subject, for example, where a second treatment occurs within 1, 2, 3, 4, 5, 6, 7 days or weeks of a first treatment. In certain embodiments, multiple doses of the modified oncolytic virus is administered to the subject over a period of 1, 2, 3, 4, 5, 6, 7 or more days or weeks. In certain embodiments, the oncolytic virus or the pharmaceutical composition as described herein is administered over a period of about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer. The frequency of administration of the oncolytic vaccinia virus or the pharmaceutical composition as described herein is, in certain instances, once daily, twice daily, once every week, once every three weeks, once every four weeks (or once a month), once every 8 weeks (or once every 2 months), once every 12 weeks (or once every 3 months), or once every 24 weeks (once every 6 months). In some embodiments of the methods disclosed herein, the oncolytic vaccinia virus or the pharmaceutical composition is administered, independently, in an initial dose for a first period of time, an intermediate dose for a second period of time, and a high dose for a third period of time. In some embodiments, the initial dose is lower than the intermediate dose and the intermediate dose is lower than the high dose. In some embodiments, the first, second, and third periods of time are, independently, about 1 week to about 2 weeks, about 2 weeks to about 3 weeks, about 3 weeks to about 4 weeks, about 4 weeks to about 5 weeks, about 6 weeks to about 7 weeks, about 7 weeks to about 8 weeks, about 8 weeks to about 9 weeks, about 9 weeks to about 10 weeks, about 10 weeks to about 11 weeks, about 11 weeks to about 12 weeks, about 12 weeks to about 24 weeks, about 24 weeks to about 48 weeks, about 48 weeks or about 52 weeks, or longer.

An exemplary method for the delivery of a modified oncolytic virus of the present disclosure, such as an oncolytic vaccinia virus as described herein or a pharmaceutical composition comprising the same, to cancer or tumor cells is via intravenous administration, e.g., via infusion, parenteral, intravenous, intradermal, intramuscular, transdermal, rectal, intraurethral, intravaginal, intranasal, intrathecal, or intraperitoneal administration. However, alternate methods of administration are also used, e.g., via intratumoral injection. The routes of administration vary with the location and nature of the tumor. In certain embodiments, the route of administration is intradental, transdermal, parenteral, intraperitoneal, intravenous, intramuscular, intranasal, subcutaneous, regional (e.g., in the proximity of a tumor, particularly with the vasculature or adjacent vasculature of a tumor), percutaneous, intrathecal, intratracheal, intraperitoneal, intraarterial, intravesical, intratumoral, inhalation, perfusion, by lavage or orally. An injectable dose of the oncolytic virus is administered as a bolus injection or as a slow infusion. In certain embodiments, the modified oncolytic virus is administered to the patient from a source implanted in the patient. In certain embodiments, administration of the modified oncolytic virus occurs by continuous infusion over a selected period of time. In some instances, an oncolytic vaccinia virus as described herein, or a pharmaceutical composition containing the same is administered at a therapeutically effective dose by infusion over a period of about 15 mins, about 30 mins, about 45 mins, about 50 mins, about 55 mins, about 60 minutes, about 75 mins, about 90 mins, about 100 mins, or about 120 mins or longer. The oncolytic virus or the pharmaceutical composition of the present disclosure is administered as a liquid dosage, wherein the total volume of administration is about 1 mL to about 5 mL, about 5 mL to 10 mL, about 15 mL to about 20 mL, about 25 mL to about 30 mL, about 30 mL to about 50 mL, about 50 mL to about 100 mL, about 100 mL to 150 mL, about 150 mL to about 200 mL, about 200 mL to about 250 mL, about 250 mL to about 300 mL, about 300 mL to about 350 mL, about 350 mL to about 400 mL, about 400 mL to about 450 mL, about 450 mL to 500 mL, about 500 mL to 750 mL, or about 750 mL to 1000 mL.

### Formulations

Pharmaceutical compositions containing a modified virus, such as an oncolytic vaccinia virus, as described herein, are prepared as solutions, dispersions in glycerol, liquid polyethylene glycols, and any combinations thereof in oils, in solid dosage forms, as inhalable dosage forms, as intranasal dosage forms, as liposomal formulations, dosage forms comprising nanoparticles, dosage forms comprising microparticles, polymeric dosage forms, or any combinations thereof. In some embodiments, a pharmaceutical composition as described herein comprises a stabilizer and a buffer. In some embodiments, a pharmaceutical composition as described herein can comprise a solubilizer, such as sterile water, Tris-buffer. In some embodiments, a pharmaceutical composition as described herein can comprise an excipient. Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In certain embodiments, a buffering agent includes phosphate buffered saline (PBS), Dulbecco's PBS (DPBS), TRIS-buffered saline (TBS), Hank's balanced salt solution (HBSS), Earle's balanced salt solution (EBSS), standard saline citrate (SSC), HEPES-buffered saline (HBS), or Gey's balanced salt solution.

In certain embodiments, a pharmaceutical composition of this disclosure comprises an effective amount of a modified virus, disclosed herein, combined with a pharmaceutically acceptable carrier. “Pharmaceutically acceptable,” as used herein, includes any carrier which does not interfere with the effectiveness of the biological activity of the active ingredients and/or that is not toxic to the patient to whom it is administered. Non-limiting examples of suitable pharmaceutical carriers include phosphate buffered saline solutions, water, emulsions, such as oil/water emulsions, various types of wetting agents and sterile solutions. Additional non-limiting examples of pharmaceutically compatible carriers include gels, bioadsorbable matrix materials, implantation elements containing the modified oncolytic virus or any other suitable vehicle, delivery or dispensing means or material. Such carriers are formulated by conventional methods and are administered to the subject at an effective amount.

### Methods of Production

The modified oncolytic viruses of this disclosure are produced by methods known to one of skill in the art. In certain embodiments, the modified oncolytic virus is propagated in suitable host cells, e.g., HeLa cells, 293 cells, or Vero cells, isolated from host cells and stored in conditions that promote stability and integrity of the virus, such that loss of infectivity over time is minimized. In certain exemplary methods, the modified oncolytic viruses are propagated in host cells using cell stacks, roller bottles, or perfusion bioreactors. In some examples, downstream methods for purification of the modified oncolytic viruses comprise filtration (e.g., depth filtration, tangential flow filtration, or a combination thereof), ultracentrifugation, or chromatographic capture. The modified oncolytic virus is stored, e.g., by freezing or drying, such as by lyophilization. In certain embodiments, prior to administration, the stored modified oncolytic virus is reconstituted (if dried for storage) and diluted in a pharmaceutically acceptable carrier for administration.

Some embodiments provide that the modified oncolytic virus as described herein, exhibit a higher titer in HeLa cells and 293 cells compared to an otherwise identical virus that does not comprise the modifications in the modified oncolytic virus. In certain instances, a higher titer in HeLa cells and 293 cells is seen in modified oncolytic virus.

### Kits

In embodiments, this disclosure provides for a kit for administering a modified oncolytic virus as described herein. In certain embodiments, a kit of this disclosure includes a modified oncolytic virus or a pharmaceutical composition comprising a modified oncolytic virus as described above. In certain embodiments, a kit of this disclosure further includes one or more components such as instructions for use, devices and additional reagents, and components, such as tubes, containers, and syringes for performing the methods disclosed above. In certain embodiments, a kit of this disclosure further includes one or more agents, e.g., at least one of an anti-cancer agent, an immunomodulatory agent, or any combinations thereof, that is administered in combination with a modified virus.

In certain embodiments, a kit of this disclosure comprises one or more containers containing a modified virus, disclosed herein. For example, and not by way of limitation, a kit of this disclosure comprises one or more containers that contain a modified oncolytic virus of this disclosure.

In certain embodiments, a kit of this disclosure includes instructions for use, a device for administering the modified oncolytic virus to a subject, or a device for administering an additional agent or compound to a subject. For example, and not by way of limitation, the instructions include a description of the modified oncolytic virus and, optionally, other components included in the kit, and methods for administration, including methods for determining the proper state of the subject, the proper dosage amount and the proper administration method for administering the modified virus. Instructions also optionally include guidance for monitoring the subject over duration of the treatment time.

In certain embodiments, a kit of this disclosure includes a device for administering the modified oncolytic virus to a subject. Any of a variety of devices known in the art for administering medications and pharmaceutical compositions is included in the kits provided herein. For example, and not by way of limitation, such devices include, a hypodermic needle, an intravenous needle, a catheter, a needle-less injection device, an inhaler, and a liquid dispenser, such as an eyedropper. In certain embodiments, a modified oncolytic virus to be delivered systemically, for example, by intravenous injection, an intratumoral injection, an intraperitoneal injection, is included in a kit with a hypodermic needle and syringe.

While preferred embodiments of this disclosure have been shown and described herein, it will be obvious to those skilled in the art that such embodiments are provided by way of example only. Numerous variations, changes, and substitutions will now occur to those skilled in the art without departing from this disclosure. It should be understood that various alternatives to the embodiments of this disclosure described herein are employed in practicing the disclosure. It is intended that the following claims define the scope of the disclosure and that methods and structures within the scope of these claims and their equivalents be covered thereby.

### Exemplary Embodiments

Provided herein are nucleic acids, wherein the nucleic acid comprises a sequence encoding for at least two polypeptides, wherein the at least two polypeptides comprise: interleukin-12 (IL-12) or a functional variant thereof; and soluble PD-1 (sPD-1) or a functional variant thereof. Further provided herein are nucleic acids, wherein a nucleic acid comprises DNA or RNA. Further provided herein are nucleic acids, wherein the IL-12 is a murine IL-12 or a human IL-12. Further provided herein are nucleic acids, wherein the IL-12 or functional variant thereof comprises an alpha subunit and a beta subunit. Further provided herein are nucleic acids, wherein the alpha subunit and the beta subunit are connected by a linker. Further provided herein are nucleic acids, wherein the sequence encoding for the IL-12 alpha subunit comprises a sequence having at least 85%, 90%, 95%, or 99% sequence identity to SEQ ID NO: 55 or SEQ ID NO: 69. Further provided herein are nucleic acids, wherein the sequence encoding for the IL-12 alpha subunit comprises a sequence of SEQ ID NO: 55 or SEQ ID NO: 69. Further provided herein are nucleic acids, wherein the sequence encoding for the IL-12 beta subunit comprises a sequence having at least 85%, 90%, 95%, or 99% sequence identity to SEQ ID NO: 53 or SEQ ID NO: 68. Further provided herein are nucleic acids, wherein the sequence encoding for the IL-12 beta subunit comprises a sequence of SEQ ID NO: 53 or SEQ ID NO: 68. Further provided herein are nucleic acids, wherein the sequence encoding for the linker comprises a sequence having at least 85%, 90%, 95%, or 99% sequence identity to SEQ ID NO: 54. Further provided herein are nucleic acids, wherein the sequence encoding for the linker comprises a sequence of SEQ ID NO: 54. Further provided herein are nucleic acids, wherein the sPD-1 comprises a domain from a murine PD-1, a domain from a human PD-1, or any combination thereof. Further provided herein are nucleic acids, wherein the sPD-1 comprises an extracellular domain of the murine PD-1, the human PD-1, or any combination thereof. Further provided herein are nucleic acids, wherein the sPD-1 comprises a domain that binds PD-L1. Further provided herein are nucleic acids, wherein the sequence encoding for the sPD-1 comprises a sequence having at least 85%, 90%, 95%, or 99% sequence identity to SEQ ID NO: 58 or SEQ ID NO: 70. Further provided herein are nucleic acids, wherein the sequence encoding for the sPD-1 comprises a sequence of SEQ ID NO: 58 or SEQ ID NO: 70. Further provided herein are nucleic acids, wherein the nucleic acid further comprises at least one promoter region. Further provided herein are nucleic acids, wherein the at least one promoter region drives expression of the at least two polypeptides. Further provided herein are nucleic acids, wherein the nucleic acid comprises a first promoter upstream to the sequence encoding the IL-12, and a second promoter upstream to the sequence encoding the sPD-1, wherein the first promoter region drives expression of the IL-12 and the second promoter region drives expression of the sPD-1. Further provided herein are nucleic acids, wherein the first promoter and the second promoter each comprises any one of PD-1 promoter, SSP, P7.5, P28, P135, 454, TK promoter, E/L, F7L, H5R, H1L, A1L, J3R, E4L, I1L, I5L, I7L, T7, I2L, FP4b, ATI, P11, PFL1, PH5, L4R, 28 kDa promoter, or any variation or combination thereof. Further provided herein are nucleic acids, wherein the first promoter comprises the P7.5 promoter. Further provided herein are nucleic acids, wherein the P7.5 promoter comprises a sequence having at least 85%, 90%, 95%, or 99% sequence identity to SEQ ID NO: 32. Further provided herein are nucleic acids, wherein the P7.5 promoter comprises a sequence of SEQ ID NO: 32. Further provided herein are nucleic acids, wherein the second promoter comprises the P28 promoter. Further provided herein are nucleic acids, wherein the P28 promoter comprises a sequence having at least 85%, 90%, 95%, or 99% sequence identity to SEQ ID NO: 29. Further provided herein are nucleic acids, wherein the P28 promoter comprises a sequence of SEQ ID NO: 29. Further provided herein are nucleic acids, wherein the nucleic acid comprises the DNA, wherein the DNA comprises sequences, in 5′ to 3′ order, of SEQ ID NO: 53, SEQ ID NO: 11, SEQ ID NO: 55, and SEQ ID NO: 58. Further provided herein are nucleic acids, wherein the nucleic acid comprises the DNA, wherein the DNA comprises sequences, in 5′ to 3′ order, of SEQ ID NO: 68, SEQ ID NO: 11, SEQ ID NO: 69, and SEQ ID NO: 70. Further provided herein are nucleic acids, wherein the nucleic acid comprises the DNA, wherein the DNA comprises sequences encoding, in 5′ to 3′ order, for SEQ ID NO: 52, and SEQ ID NO: 58. Further provided herein are nucleic acids, wherein the nucleic acid comprises the DNA, wherein the DNA comprises sequences encoding, in 5′ to 3′ order, for SEQ ID NO: 67, and SEQ ID NO: 70. Further provided herein are nucleic acids, wherein the nucleic acid comprises the DNA, wherein the DNA comprises a sequence of SEQ ID NO: 66. Further provided herein are nucleic acids, wherein the nucleic acid comprises the DNA, wherein the DNA comprises a sequence of SEQ ID NO: 71. Further provided herein are nucleic acids, further comprising a sequence encoding for a chemokine receptor or a functional variant thereof. Further provided herein are nucleic acids, wherein the chemokine receptor comprises at least one of: a CXC receptor, a CC receptor, a CX3C receptor, an XC receptor, a functional fragment thereof, a functional variant thereof, or any combinations thereof. Further provided herein are nucleic acids, wherein the chemokine receptor comprises at least one of: CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, XCR1, a functional fragment thereof or a functional variant thereof, or any combinations thereof.

Provided herein are nucleic acids, wherein the nucleic acid comprises: a first region encoding a first polypeptide comprising a sequence having at least 85%, 90%, 95%, or 99% sequence identity to SEQ ID NO: 5 or SEQ ID NO: 8; and a second region encoding a second polypeptide comprising a sequence having at least 85%, 90%, 95%, or 99% sequence identity to SEQ ID NO: 2 or SEQ ID NO: 4. Further provided herein are nucleic acids, wherein the first polypeptide comprises a sequence of SEQ ID NO: 5 or SEQ ID NO: 8. Further provided herein are nucleic acids, wherein the second polypeptide comprises a sequence of SEQ ID NO: 2 or SEQ ID NO: 4. Further provided herein are nucleic acids, wherein the second polypeptide comprising the sequence of SEQ ID NO: 4, further comprises one or more substitutions as described by E61V, M70I, Q75F, K78W, K78L, E84F, S87W, A129H, A132L, K135M. Further provided herein are nucleic acids, further comprising at least one promoter region. Further provided herein are nucleic acids, wherein the at least one promoter region drives expression of the first region and the second region. Further provided herein are nucleic acids, wherein the nucleic acid comprises a first promoter that drives expression of the first region and a second promoter that drives expression of the second region. Further provided herein are nucleic acids, wherein the first promoter and the second promoter each comprises any one of SSP, P7.5, P28, P135, 454, TK promoter, E/L, F7L, H5R, H1L, A1L, J3R, E4L, I1L, I5L, I7L, T7, 12L, FP4b, ATI, P11, PFL1, PH5, L4R, 28 kDa promoter, or any variation or combination thereof. Further provided herein are nucleic acids, wherein the first promoter comprises the P7.5 promoter. Further provided herein are nucleic acids, wherein the second promoter comprises the P28 promoter. Further provided herein are nucleic acids, wherein: the first region encodes a polypeptide comprising the sequence of SEQ ID NO: 5 or SEQ ID NO: 8; and the second region encodes a polypeptide comprising the sequence of SEQ ID NO: 2 or SEQ ID NO: 4. Further provided herein are nucleic acids, further comprising a sequence encoding for a chemokine receptor or a functional variant thereof. Further provided herein are nucleic acids, wherein the chemokine receptor comprises at least one of: a CXC receptor, a CC receptor, a CX3C receptor, an XC receptor, a functional fragment thereof, a functional variant thereof, or any combinations thereof. Further provided herein are nucleic acids, wherein the chemokine receptor comprises at least one of: CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, XCR1, a functional fragment thereof or a functional variant thereof, or any combinations thereof.

Provided herein are nucleic acids as described herein, wherein the nucleic acid is present in an oncolytic virus. Further provided herein are nucleic acids, wherein the oncolytic virus is a poxvirus, an adeno associated virus, an adenovirus, a reovirus, a lentivirus, a herpes simplex virus, a vesicular stomatitis virus, a mengovirus, a myxoma virus, Newcastle disease virus, measles virus, or polio virus. Further provided herein are nucleic acids, wherein the poxvirus is a vaccinia virus. Further provided herein are nucleic acids, wherein the vaccinia virus is a modified strain of Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325). Further provided herein are nucleic acids, wherein the nucleic acid is inserted into the viral genome. Further provided herein are nucleic acids, further comprising a mutation or deletion of at least one viral gene selected from the group consisting of: Thymidine Kinase (TK), F13L, A36R, A34R, A33R, A52R, B5R, B8R, B18R, SPI-1, SPI-2, B15R, VGF, E3L, K3L, A41L, K7R, or N1L, a functional fragment thereof, or any combinations thereof. Further provided herein are nucleic acids, comprising a mutation or deletion of the TK gene. Further provided herein are nucleic acids, further comprising a mutation or deletion of the B8R gene.

Provided herein are compositions, wherein the composition comprises: a vector; an exogenous nucleic acid comprising a sequence encoding for a cytokine or a functional variant thereof; and an exogenous nucleic acid comprising a sequence encoding for a PD-L1 receptor. Further provided herein are compositions, Further provided herein are compositions, wherein the cytokine comprises IL-12 or a functional variant thereof. Further provided herein are compositions, wherein the IL-12 comprises a sequence having at least 85%, 90%, 95%, or 99% sequence identity to SEQ ID NO: 5 or SEQ ID NO: 8. Further provided herein are compositions, wherein the IL-12 comprises a sequence of SEQ ID NO: 5 or SEQ ID NO: 8. Further provided herein are compositions, wherein the PD-L1 receptor comprises a PD-1 dominant negative, a soluble PD-1 dominant negative, or a protein that binds PD-L1. Further provided herein are compositions, wherein the protein that binds PD-L1 is an anti-PD-L1 antibody. Further provided herein are compositions, wherein the protein that binds PD-L1 is a soluble variant of PD-1 (sPD-1). Further provided herein are compositions, wherein the sPD-1 comprises a sequence having at least 85%, 90%, 95%, or 99% sequence identity to SEQ ID NO: 2 or SEQ ID NO: 4. Further provided herein are compositions, wherein the sPD-1 comprises a sequence of either SEQ ID NO: 2 or SEQ ID NO: 4. Further provided herein are compositions, wherein the sPD-1 comprising the sequence of SEQ ID NO: 4, further comprises one or more substitutions as described by E61V, M70I, Q75F, K78W, K78L, E84F, S87W, A129H, A132L, K135M. Further provided herein are compositions, further comprising at least one promoter region. Further provided herein are compositions, wherein the at least one promoter region drives expression of the cytokine and the PD-L1 receptor. Further provided herein are compositions, wherein the at least one promoter region comprises a first promoter that drives expression of the cytokine, and a second promoter that drives expression of the PD-L1 receptor. Further provided herein are compositions, wherein the at least one promoter comprises any one of SSP, P7.5, P28, P135, 454, TK promoter, E/L, F7L, H5R, H1L, A1L, J3R, E4L, I1L, I5L, I7L, T7, I2L, FP4b, ATI, P11, PFL1, PH5, L4R, 28 kDa promoter, or any variation or combination thereof. Further provided herein are compositions, wherein the first promoter comprises the P7.5 promoter. Further provided herein are compositions, wherein the second promoter comprises the P28 promoter. Further provided herein are compositions, wherein the exogenous nucleic acids comprise a combined sequence of SEQ ID NO: 66. Further provided herein are compositions, wherein the exogenous nucleic acids comprise a combines sequence of SEQ ID NO: 71. Further provided herein are compositions, further comprising an exogenous nucleic acid comprising a sequence encoding for a chemokine receptor or a functional variant thereof. Further provided herein are compositions, wherein the chemokine receptor comprises at least one of: a CXC receptor, a CC receptor, a CX3C receptor, an XC receptor, a functional fragment thereof, a functional variant thereof, or any combinations thereof. Further provided herein are compositions, wherein the chemokine receptor comprises at least one of: CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, XCR1, a functional fragment thereof or a functional variant thereof, or any combinations thereof. Further provided herein are compositions, wherein the vector comprises a plasmid, a phage, a virus, a cosmid, an artificial chromosome, a liposome, a nanoparticle, or any combination thereof. Further provided herein are compositions, wherein the vector is an oncolytic virus. Further provided herein are compositions, wherein the oncolytic virus is a poxvirus, an adeno associated virus, an adenovirus, a reovirus, a lentivirus, a herpes simplex virus, a vesicular stomatitis virus, a mengovirus, a myxoma virus, Newcastle disease virus, measles virus, or polio virus. Further provided herein are compositions, wherein the poxvirus is a vaccinia virus. Further provided herein are compositions, wherein the vaccinia virus is a modified strain of Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325). Further provided herein are compositions, wherein the exogenous nucleic acids are inserted into the viral genome. Further provided herein are compositions, wherein the oncolytic virus comprises at least one genome modification. Further provided herein are compositions, wherein the at least one modification comprises a mutation or deletion of at least one gene selected from the group consisting of: Thymidine Kinase, F13L, A36R, A34R, A33R, A52R, B5R, B8R, B18R, SPI-1, SPI-2, B15R, VGF, E3L, K3L, A41L, K7R, or NIL, a functional fragment thereof, or any combinations thereof. Further provided herein are compositions, comprising a mutation or deletion of the TK gene. Further provided herein are compositions, further comprising a mutation or deletion of the B8R gene.

Provided herein are oncolytic viruses, wherein the oncolytic virus comprises: an insertion at a TK gene locus comprising, in 5′ to 3′ order: a first promoter region, wherein the promoter is P7.5; a first region encoding IL-12; a second promoter region, wherein the promoter is P728; and a second region encoding a PD-1 variant.

Provided herein are pharmaceutical compositions, wherein the pharmaceutical composition comprises: the nucleic acid as described herein or the composition as described herein; and a pharmaceutically acceptable excipient. Further provided herein are pharmaceutical compositions, wherein the composition is in a liquid dosage form. Further provided herein are pharmaceutical compositions, wherein the pharmaceutically acceptable excipient is a buffered saline. Further provided herein are pharmaceutical compositions, wherein the buffered saline is phosphate buffered saline (PBS), Dulbecco's PBS (DPBS), TRIS-buffered saline (TBS), Hank's balanced salt solution (HBSS), Earle's balanced salt solution (EBSS), standard saline citrate (SSC), HEPES-buffered saline (HBS), or Gey's balanced salt solution. Further provided herein are pharmaceutical compositions, wherein the composition further comprises a liposome or nanoparticle. Further provided herein are pharmaceutical compositions, wherein the nucleic acid or vector is associated with the liposome or nanoparticle.

Provided herein are methods for treatment of cancer comprising administering to a subject having cancer the pharmaceutical composition as described herein in an amount sufficient for treatment of a cancer. Further provided herein are methods for treatment of cancer, wherein the cancer is a solid tumor, a leukemia, or a lymphoma. Further provided herein are methods for treatment of cancer, wherein the cancer comprises melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, or sarcoma. Further provided herein are methods for treatment of cancer, wherein the administering comprises an intratumoral administration. Further provided herein are methods for treatment of cancer, wherein the administering comprises a systemic administration. Further provided herein are methods for treatment of cancer, wherein the systemic administration comprises oral administration, parenteral administration, intranasal administration, sublingual administration, rectal administration, transdermal administration, or any combinations thereof.

Provided herein are methods for activating an anti-tumor immune response, comprising administering to a subject having a cancer the pharmaceutical composition described herein. Further provided herein are methods for activating an anti-tumor immune response, wherein the cancer is a solid tumor, a leukemia, or a lymphoma. Further provided herein are methods for activating an anti-tumor immune response, wherein the cancer comprises melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, or sarcoma. Further provided herein are methods for activating an anti-tumor immune response, wherein the administering step is an intratumoral administration. Further provided herein are methods for activating an anti-tumor immune response, wherein the administering step is a systemic administration. Further provided herein are methods for activating an anti-tumor immune response, wherein the systemic administration comprises oral administration, parenteral administration, intranasal administration, sublingual administration, rectal administration, transdermal administration, or any combinations thereof.

Provided herein are methods for reduction of incidence of tumor cell growth, comprising: administering to tumor cells the pharmaceutical composition as described herein in an effective amount sufficient for reduction of incidence of tumor cell growth. Further provided herein are methods for reduction of incidence of tumor cell growth, wherein the tumor cells are from a solid, a leukemia, or a lymphoma. Further provided herein are methods for reduction of incidence of tumor cell growth, wherein the tumor cells are from a melanoma, hepatocellular carcinoma, breast cancer, lung cancer, peritoneal cancer, prostate cancer, bladder cancer, ovarian cancer, leukemia, lymphoma, renal carcinoma, pancreatic cancer, epithelial carcinoma, gastric cancer, colon carcinoma, duodenal cancer, pancreatic adenocarcinoma, mesothelioma, glioblastoma multiforme, astrocytoma, multiple myeloma, prostate carcinoma, hepatocellular carcinoma, cholangiosarcoma, pancreatic adenocarcinoma, head and neck squamous cell carcinoma, colorectal cancer, intestinal-type gastric adenocarcinoma, cervical squamous-cell carcinoma, osteosarcoma, epithelial ovarian carcinoma, acute lymphoblastic lymphoma, myeloproliferative neoplasms, or sarcoma. Further provided herein are methods for reduction of incidence of tumor cell growth, wherein the administering step is an intratumoral administration.

### EXAMPLES

The examples below further illustrate the described embodiments without limiting the scope of this disclosure.

### Example 1: Construction of Murine IL-12 and Murine SPD-1 Expression System

A vaccinia virus, Western Reserve strain, was modified by replacing the gene encoding thymidine kinase (VACV094, J2R) with nucleic acids encoding a murine IL-12 (mIL-12), a soluble murine PD-1 (murine sPD-1), and a fluorescent reporter. A plasmid transfer vector was generated comprising, in order, with no gaps, a 5′ recombination directing sequence (SEQ ID NO: 47), an SbfI cloning site (CCTGCAGG), a P7.5 promoter (SEQ ID NO: 44), a KpnI cloning site (GGTACC), open reading frame encoding murine IL-12 (SEQ ID NO: 48), SalI cloning site followed by spacer (SEQ ID NO: 52), a P28 promoter (SEQ ID NO: 41), a SpeI cloning site (ACTAGT), soluble murine PD-1 (SEQ ID NO: 53), a sacI cloning site (GAGCTC), a loxP sequence (SEQ ID NO: 54), a spacer sequence A (SEQ ID NO: 55), viral 454 promoter (SEQ ID NO: 34), fluorescent reporter protein (SEQ ID NO: 56), a PacI cloning site (TTAATTAA), a short spacer B (SEQ ID NO: 57), a loxP sequence (SEQ ID NO: 54), and a 3′ recombination directing sequence (SEQ ID NO: 58). Following recombination and treatment with cre recombinase, the viral genome comprised the incorporated sequence of SEQ ID NO: 59. Selected sequences are shown in Table 6. A schematic representation of the promoter and transgene inserted at a TK locus is shown in FIG. 2.

TABLE 6
Murine IL-12 and sPD-1 recombination sequences.

| SEQ | | |
|---|---|---|
| ID NO: | Description | Nucleic acid sequence |
| 47 | 5′ recombination-directing | ATTTACGGATTCACCAATAAAAATAAACTAGAG |
| | sequence | AAACTTAGTACTAATAAGGAACTAGAATCGTAT |
| | | AGTTCTAGCCCTCTTCAAGAACCCATTAGGTTAA |
| | | ATGATTTTCTGGGACTATTGGAATGTGTTAAAAA |
| | | GAATATTCCTCTAACAGATATTCCGACAAAGGA |
| | | TTGATTACTATAAATGGAGAATGTTCCTAATGTA |
| | | TACTTTAATCCTGTGTTTATAGAGCCCACGTTTA |
| | | AACATTCTTTATTAAGTGTTTATAAACACAGATT |
| | | AATAGTTTTATTTGAAGTATTCGTTGTATTCATTC |
| | | TAATATATGTATTTTTTAGATCTGAATTAAATAT |
| | | GTTCTTCATGCCTAAACGAAAAATACCCGATCCT |
| | | ATTGATAGATTACGACGTGCTAATCTAGCGTGTG |
| | | AAGACGATAAATTAATGATCTATGGATTACCAT |
| | | GGATGACAACTCAAACATCTGCGTTATCAATAA |
| | | ATAGTAAACCGATAGTGTATAAAGATTGTGCAA |
| | | AGCTTTTGCGATCAATAAATGGATCACAACCAG |
| | | TATCTCTTAACGATGTTCTTCGCAGATGATGATT |
| | | CATTTTTTAAGTATTTGGCTAGTCAAGATGATGA |
| | | ATCTTCATTATCTGATATATTGCAAATCACTCAA |
| | | TATCTAGACTTTCTGTTATTATTATTGATCCAATC |
| | | AAAAAATAAATTAGAAGCCGTGGGTCATTGTTA |
| | | TGAATCTCTTTCAGAGGAATACAGACAATTGAC |
| | | AAAATTCACAGACTTTCAAGATTTTAAAAAACT |
| | | GTTTAACAAGGTCCCTATTGTTACAGATGGAAG |
| | | GGTCAAACTTAATAAAGGATATTTGTTCGACTTT |
| | | GTGATTAGTTTGATGCGATTCAAAAAAGAATCCT |
| | | CTCTAGCTACCACCGCAATAGATCCTGTTAGATA |
| | | CATAGATCCTCGTCGCAATATCGCATTTTCTAAC |
| | | GTGATGGATATATTAAAGTCGAATAAAGTGAAC |
| | | AATAATTAATTCTTTATTGTCATC |
| | SbfI cloning site | CCTGCAGG |
| | KpnI cloning site | GGTACC |
| 48 | murine IL-12 | ATGTGTCCTCAGAAGCTAACCATCTCCTGGTTTG |
| | | CCATCGTTTTGCTGGTGTCTCCACTCATGGCCAT |
| | | GTGGGAGCTGGAGAAAGACGTTTATGTTGTAGA |
| | | GGTGGACTGGACTCCCGATGCCCCTGGAGAAAC |
| | | AGTGAACCTCACCTGTGACACGCCTGAAGAAGA |
| | | TGACATCACCTGGACCTCAGACCAGAGACATGG |
| | | AGTCATAGGCTCTGGAAAGACCCTGACCATCAC |
| | | TGTCAAAGAGTTTCTAGATGCTGGCCAGTACACC |
| | | TGCCACAAAGGAGGCGAGACTCTGAGCCACTCA |
| | | CATCTGCTGCTCCACAAGAAGGAAAATGGAATT |
| | | TGGTCCACTGAAATTTTAAAAAATTTCAAAAAC |
| | | AAGACTTTCCTGAAGTGTGAAGCACCAAATTAC |
| | | TCCGGACGGTTCACGTGCTCATGGCTGGTGCAA |
| | | AGAAACATGGACTTGAAGTTCAACATCAAGAGC |
| | | AGTAGCAGTTCCCCTGACTCTCGGGCAGTGACAT |
| | | GTGGAATGGCGTCTCTGTCTGCAGAGAAGGTCA |
| | | CACTGGACCAAAGGGACTATGAGAAGTATTCAG |
| | | TGTCCTGCCAGGAGGATGTCACCTGCCCAACTGC |
| | | CGAGGAGACCCTGCCCATTGAACTGGCGTTGGA |
| | | AGCACGGCAGCAGAATAAATATGAGAACTACAG |
| | | CACCAGCTTCTTCATCAGGGACATCATCAAACCA |
| | | GACCCGCCCAAGAACTTGCAGATGAAGCCTTTG |
| | | AAGAACTCACAGGTGGAGGTCAGCTGGGAGTAC |
| | | CCTGACTCCTGGAGCACTCCCCATTCCTACTTCT |
| | | CCCTCAAGTTCTTTGTTCGAATCCAGCGCAAGAA |
| | | AGAAAAGATGAAGGAGACAGAGGAGGGGTGTA |
| | | ACCAGAAAGGTGCGTTCCTCGTAGAGAAGACAT |
| | | CTACCGAAGTCCAATGCAAAGGCGGGAATGTCT |
| | | GCGTGCAAGCTCAGGATCGCTATTACAATTCCTC |
| | | ATGCAGCAAGTGGGCATGTGTTCCATGCAGGGT |
| | | CCGATCCGGAGGTGGCGGATCCGGCGGAGGTGG |
| | | CTCAGGTGGCGGTGGATCAGGAGGAGGTGGATC |
| | | AGCTAGCGCCACCCTTGCCCTCCTAAACCACCTC |
| | | AGTTTGGCCAGGGTCATTCCAGTCTCTGGACCTG |
| | | CCAGGTGTCTTAGCCAGTCCCGAAACCTGCTGA |
| | | AGACCACAGATGACATGGTGAAGACGGCCAGAG |
| | | AAAAACTGAAACATTATTCCTGCACTGCTGAAG |
| | | ACATCGACCATGAAGACATCACACGGGACCAAA |
| | | CCAGCACATTGAAGACCTGTTTACCACTGGAACT |
| | | ACACAAGAACGAGAGTTGCCTGGCTACTAGAGA |
| | | GACTTCTTCCACAACAAGAGGGAGCTGCCTGCC |
| | | CCCACAGAAGACGTCTTTGATGATGACCCTGTGC |
| | | CTTGGTAGCATCTATGAGGACTTGAAGATGTACC |
| | | AGACAGAGTTCCAGGCCATCAACGCAGCACTTC |
| | | AGAATCACAACCATCAGCAGATCATTCTAGACA |
| | | AGGGCATGCTGGTGGCCATCGACGAGCTGATGC |
| | | AGTCTCTGAATCATAATGGCGAGACTCTGCGCC |
| | | AGAAACCTCCTGTGGGAGAAGCAGACCCTTACA |
| | | GAGTGAAAATGAAGCTCTGCATCCTGCTTCACG |
| | | CCTTCAGCACCCGCGTCGTGACCATCAACAGGG |
| | | TGATGGGCTATCTGAGTTCCGCCTAA |
| 49 | murine IL-12b | ATGTGTCCTCAGAAGCTAACCATCTCCTGGTTTG |
| | | CCATCGTTTTGCTGGTGTCTCCACTCATGGCCAT |
| | | GTGGGAGCTGGAGAAAGACGTTTATGTTGTAGA |
| | | GGTGGACTGGACTCCCGATGCCCCTGGAGAAAC |
| | | AGTGAACCTCACCTGTGACACGCCTGAAGAAGA |
| | | TGACATCACCTGGACCTCAGACCAGAGACATGG |
| | | AGTCATAGGCTCTGGAAAGACCCTGACCATCAC |
| | | TGTCAAAGAGTTTCTAGATGCTGGCCAGTACACC |
| | | TGCCACAAAGGAGGCGAGACTCTGAGCCACTCA |
| | | CATCTGCTGCTCCACAAGAAGGAAAATGGAATT |
| | | TGGTCCACTGAAATTTTAAAAAATTTCAAAAAC |
| | | AAGACTTTCCTGAAGTGTGAAGCACCAAATTAC |
| | | TCCGGACGGTTCACGTGCTCATGGCTGGTGCAA |
| | | AGAAACATGGACTTGAAGTTCAACATCAAGAGC |
| | | AGTAGCAGTTCCCCTGACTCTCGGGCAGTGACAT |
| | | GTGGAATGGCGTCTCTGTCTGCAGAGAAGGTCA |
| | | CACTGGACCAAAGGGACTATGAGAAGTATTCAG |
| | | TGTCCTGCCAGGAGGATGTCACCTGCCCAACTGC |
| | | CGAGGAGACCCTGCCCATTGAACTGGCGTTGGA |
| | | AGCACGGCAGCAGAATAAATATGAGAACTACAG |
| | | CACCAGCTTCTTCATCAGGGACATCATCAAACCA |
| | | GACCCGCCCAAGAACTTGCAGATGAAGCCTTTG |
| | | AAGAACTCACAGGTGGAGGTCAGCTGGGAGTAC |
| | | CCTGACTCCTGGAGCACTCCCCATTCCTACTTCT |
| | | CCCTCAAGTTCTTTGTTCGAATCCAGCGCAAGAA |
| | | AGAAAAGATGAAGGAGACAGAGGAGGGGTGTA |
| | | ACCAGAAAGGTGCGTTCCTCGTAGAGAAGACAT |
| | | CTACCGAAGTCCAATGCAAAGGCGGGAATGTCT |
| | | GCGTGCAAGCTCAGGATCGCTATTACAATTCCTC |
| | | ATGCAGCAAGTGGGCATGTGTTCCATGCAGGGT |
| | | CCGATCC |
| 50 | linker | GGAGGTGGCGGATCCGGCGGAGGTGGCTCAGGT |
| | | GGCGGTGGATCAGGAGGAGGTGGATCAGCTAGC |
| 51 | murine IL-12a | GCCACCCTTGCCCTCCTAAACCACCTCAGTTTGG |
| | | CCAGGGTCATTCCAGTCTCTGGACCTGCCAGGTG |
| | | TCTTAGCCAGTCCCGAAACCTGCTGAAGACCAC |
| | | AGATGACATGGTGAAGACGGCCAGAGAAAAACT |
| | | GAAACATTATTCCTGCACTGCTGAAGACATCGA |
| | | CCATGAAGACATCACACGGGACCAAACCAGCAC |
| | | ATTGAAGACCTGTTTACCACTGGAACTACACAA |
| | | GAACGAGAGTTGCCTGGCTACTAGAGAGACTTC |
| | | TTCCACAACAAGAGGGAGCTGCCTGCCCCCACA |
| | | GAAGACGTCTTTGATGATGACCCTGTGCCTTGGT |
| | | AGCATCTATGAGGACTTGAAGATGTACCAGACA |
| | | GAGTTCCAGGCCATCAACGCAGCACTTCAGAAT |
| | | CACAACCATCAGCAGATCATTCTAGACAAGGGC |
| | | ATGCTGGTGGCCATCGACGAGCTGATGCAGTCT |
| | | CTGAATCATAATGGCGAGACTCTGCGCCAGAAA |
| | | CCTCCTGTGGGAGAAGCAGACCCTTACAGAGTG |
| | | AAAATGAAGCTCTGCATCCTGCTTCACGCCTTCA |
| | | GCACCCGCGTCGTGACCATCAACAGGGTGATGG |
| | | GCTATCTGAGTTCCGCCTAA |
| 52 | SalI cloning site followed by | GTCGACTGAGTGACCGTGA |
| | short spacer sequence | |
| | SpeI cloning site | ACTAGT |
| 53 | soluble murine PD-1 | ATGTGGGTCCGGCAAGTACCCTGGTCATTCACTT |
| | | GGGCTGTGCTGCAGTTGAGCTGGCAATCAGGGT |
| | | GGCTTCTAGAGGTCCCCAATGGGCCCTGGAGGT |
| | | CCCTCACCTTCTACCCAGCCTGGCTCACAGTGTC |
| | | AGAGGGAGCAAATGCCACCTTCACCTGCAGCTT |
| | | GTCCAACTGGTCGGAGGATCTTATGCTGAACTG |
| | | GAACCGCCTGAGTCCCAGCAACCAGACTGAAAA |
| | | ACAGGCCGCCTTCTGTAATGGTTTGAGCCAACCC |
| | | GTCCAGGATGCCCGCTTCCAGATCATACAGCTGC |
| | | CCAACAGGCATGACTTCCACATGAACATCCTTG |
| | | ACACACGGCGCAATGACAGTGGCATCTACCTCT |
| | | GTGGGGCCATCTCCCTGCACCCCAAGGCAAAAA |
| | | TCGAGGAGAGCCCTGGAGCAGAGCTAGTGGTAA |
| | | CAGAGAGAATCCTGGAGACCTCAACAAGATATC |
| | | CCAGCCCCTCGCCCAAACCAGAAGGCCGGTTTC |
| | | AAGGCTAG |
| | SacI cloning site | GAGCTC |
| 54 | LoxP sequence | ATAACTTCGTATAGCATACATTATACGAAGTTAT |
| 55 | Spacer sequence A | AATTCTGCAGAT |
| 56 | Fluorescent reporter protein | ATGCGTAAAGGCGAAGAGCTGTTCACTGGTGTC |
| | | GTCCCTATTCTGGTGGAACTGGATGGTGATGTCA |
| | | ACGGTCATAAGTTTTCCGTGCGTGGCGAGGGTG |
| | | AAGGTGACGCAACTAATGGTAAACTGACGCTGA |
| | | AGTTCATCTGTACTACTGGTAAACTGCCGGTCCC |
| | | TTGGCCGACTCTGGTAACGACGCTGACTTATGGT |
| | | GTTCAGTGCTTTGCTCGTTATCCGGACCATATGA |
| | | AGCAGCATGACTTCTTCAAGTCCGCCATGCCGG |
| | | AAGGCTATGTGCAGGAACGCACGATTTCCTTTA |
| | | AGGATGACGGCACGTACAAAACGCGTGCGGAAG |
| | | TGAAATTTGAAGGCGATACCCTGGTAAACCGCA |
| | | TTGAGCTGAAAGGCATTGACTTTAAAGAAGACG |
| | | GCAATATCCTGGGCCATAAGCTGGAATACAATT |
| | | TTAACAGCCACAATGTTTACATCACCGCCGATAA |
| | | ACAAAAAAATGGCATTAAAGCGAATTTTAAAAT |
| | | TCGCCACAACGTGGAGGATGGCAGCGTGCAGCT |
| | | GGCTGATCACTACCAGCAAAACACTCCAATCGG |
| | | TGATGGTCCTGTTCTGCTGCCAGACAATCACTAT |
| | | CTGAGCACGCAAAGCGTTCTGTCTAAAGATCCG |
| | | AACGAGAAACGCGATCATATGGTTCTGCTGGAG |
| | | TTCGTAACCGCAGCGGGCATCACGCATGGTATG |
| | | GATGAACTGTACAAATAA |
| | PacI cloning site | TTAATTAA |
| 57 | Spacer sequence B | AAGGCCGCTCGA |
| 58 | Homologous recombination- | TATTATATTTTTTATCTAAAAAACTAAAAATAAA |
| | directing sequence, 3′ | CATTGATTAAATTTTAATATAATACTTAAAAATG |
| | | GATGTTGTGTCGTTAGATAAACCGTTTATGTATT |
| | | TTGAGGAAATTGATAATGAGTTAGATTACGAAC |
| | | CAGAAAGTGCAAATGAGGTCGCAAAAAAACTGC |
| | | CGTATCAAGGACAGTTAAAACTATTACTAGGAG |
| | | AATTATTTTTTCTTAGTAAGTTACAGCGACACGG |
| | | TATATTAGATGGTGCCACCGTAGTGTATATAGGA |
| | | TCTGCTCCCGGTACACATATACGTTATTTGAGAG |
| | | ATCATTTCTATAATTTAGGAGTGATCATCAAATG |
| | | GATGCTAATTGACGGCCGCCATCATGATCCTATT |
| | | TTAAATGGATTGCGTGATGTGACTCTAGTGACTC |
| | | GGTTCGTTGATGAGGAATATCTACGATCCATCAA |
| | | AAAACAACTGCATCCTTCTAAGATTATTTTAATT |
| | | TCTGATGTGAGATCCAAACGAGGAGGAAATGAA |
| | | CCTAGTACGGCGGATTTACTAAGTAATTACGCTC |
| | | TACAAAATGTCATGATTAGTATTTTAAACCCCGT |
| | | GGCGTCTAGTCTTAAATGGAGATGCCCGTTTCCA |
| | | GATCAATGGATCAAGGACTTTTATATCCCACACG |
| | | GTAATAAAATGTTACAACCTTTTGCTCCTTCATA |
| | | TTCAGCTGAAATGAGATTATTAAGTATTTATACC |
| | | GGTGAGAACATGAGACTGACTCGAGTTACCAAA |
| | | TCAGACGCTGTAAATTATGAAAAAAAGATGTAC |
| | | TACCTTAATAAGATCGTCCGTAACAAAGTAGTTG |
| | | TTAACTTTGATTATCCTAATCAGGAATATGACTA |
| | | TTTTCACATGTACTTTATGCTGAGGACCGTGTAC |
| | | TGCAATAAAACATTTCCTACTACTAAAGCAAAG |
| | | GTACTATTTCTACAACAATCTATATTTCGTTTCTT |
| | | AAATATTCCAACAACATCAACTGAAAAAGTTAG |
| | | TCATGAACCAATACAACG |
| 59 | Final sequence in viral genome | ATTTACGGATTCACCAATAAAAATAAACTAGAG |
| | (non-native DNA BOLD; | AAACTTAGTACTAATAAGGAACTAGAATCGTAT |
| | transgene open reading frames | AGTTCTAGCCCTCTTCAAGAACCCATTAGGTTAA |
| | UNDERLINED) | ATGATTTTCTGGGACTATTGGAATGTGTTAAAAA |
| | | GAATATTCCTCTAACAGATATTCCGACAAAGGA |
| | | TTGATTACTATAAATGGAGAATGTTCCTAATGTA |
| | | TACTTTAATCCTGTGTTTATAGAGCCCACGTTTA |
| | | AACATTCTTTATTAAGTGTTTATAAACACAGATT |
| | | AATAGTTTTATTTGAAGTATTCGTTGTATTCATTC |
| | | TAATATATGTATTTTTTAGATCTGAATTAAATAT |
| | | GTTCTTCATGCCTAAACGAAAAATACCCGATCCT |
| | | ATTGATAGATTACGACGTGCTAATCTAGCGTGTG |
| | | AAGACGATAAATTAATGATCTATGGATTACCAT |
| | | GGATGACAACTCAAACATCTGCGTTATCAATAA |
| | | ATAGTAAACCGATAGTGTATAAAGATTGTGCAA |
| | | AGCTTTTGCGATCAATAAATGGATCACAACCAG |
| | | TATCTCTTAACGATGTTCTTCGCAGATGATGATT |
| | | CATTTTTTAAGTATTTGGCTAGTCAAGATGATGA |
| | | ATCTTCATTATCTGATATATTGCAAATCACTCAA |
| | | TATCTAGACTTTCTGTTATTATTATTGATCCAATC |
| | | AAAAAATAAATTAGAAGCCGTGGGTCATTGTTA |
| | | TGAATCTCTTTCAGAGGAATACAGACAATTGAC |
| | | AAAATTCACAGACTTTCAAGATTTTAAAAAACT |
| | | GTTTAACAAGGTCCCTATTGTTACAGATGGAAG |
| | | GGTCAAACTTAATAAAGGATATTTGTTCGACTTT |
| | | GTGATTAGTTTGATGCGATTCAAAAAAGAATCCT |
| | | CTCTAGCTACCACCGCAATAGATCCTGTTAGATA |
| | | CATAGATCCTCGTCGCAATATCGCATTTTCTAAC |
| | | GTGATGGATATATTAAAGTCGAATAAAGTGAAC |
| | | AATAATTAATTCTTTATTGTCATCCCTGCAGGTC |
| | | ACTAATTCCAAACCCACCCGCTTTTTATAGTA |
| | | AGTTTTTCACCCATAAATAATAAATACAATAA |
| | | TTAATTTCTCGTAAAAGTAGAAAATATATTCT |
| | | AATTTATTGCACGGTAAGGAAGTAGATCATAA |
| | | GGTACCATGTGTCCTCAGAAGCTAACCATCTC |
| | | CTGGTTTGCCATCGTTTTGCTGGTGTCTCCAC |
| | | TCATGGCCATGTGGGAGCTGGAGAAAGACGT |
| | | TTATGTTGTAGAGGTGGACTGGACTCCCGAT |
| | | GCCCCTGGAGAAACAGTGAACCTCACCTGTG |
| | | ACACGCCTGAAGAAGATGACATCACCTGGAC |
| | | CTCAGACCAGAGACATGGAGTCATAGGCTCT |
| | | GGAAAGACCCTGACCATCACTGTCAAAGAGT |
| | | TTCTAGATGCTGGCCAGTACACCTGCCACAAA |
| | | GGAGGCGAGACTCTGAGCCACTCACATCTGC |
| | | TGCTCCACAAGAAGGAAAATGGAATTTGGTC |
| | | CACTGAAATTTTAAAAAATTTCAAAAACAAGA |
| | | CTTTCCTGAAGTGTGAAGCACCAAATTACTCC |
| | | GGACGGTTCACGTGCTCATGGCTGGTGCAAA |
| | | GAAACATGGACTTGAAGTTCAACATCAAGAG |
| | | CAGTAGCAGTTCCCCTGACTCTCGGGCAGTG |
| | | ACATGTGGAATGGCGTCTCTGTCTGCAGAGA |
| | | AGGTCACACTGGACCAAAGGGACTATGAGAA |
| | | GTATTCAGTGTCCTGCCAGGAGGATGTCACCT |
| | | GCCCAACTGCCGAGGAGACCCTGCCCATTGA |
| | | ACTGGCGTTGGAAGCACGGCAGCAGAATAAA |
| | | TATGAGAACTACAGCACCAGCTTCTTCATCAG |
| | | GGACATCATCAAACCAGACCCGCCCAAGAAC |
| | | TTGCAGATGAAGCCTTTGAAGAACTCACAGGT |
| | | GGAGGTCAGCTGGGAGTACCCTGACTCCTGG |
| | | AGCACTCCCCATTCCTACTTCTCCCTCAAGTT |
| | | CTTTGTTCGAATCCAGCGCAAGAAAGAAAAG |
| | | ATGAAGGAGACAGAGGAGGGGTGTAACCAGA |
| | | AAGGTGCGTTCCTCGTAGAGAAGACATCTAC |
| | | CGAAGTCCAATGCAAAGGCGGGAATGTCTGC |
| | | GTGCAAGCTCAGGATCGCTATTACAATTCCTC |
| | | ATGCAGCAAGTGGGCATGTGTTCCATGCAGG |
| | | GTCCGATCCGGAGGTGGCGGATCCGGCGGAG |
| | | GTGGCTCAGGTGGCGGTGGATCAGGAGGAGG |
| | | TGGATCAGCTAGCGCCACCCTTGCCCTCCTAA |
| | | ACCACCTCAGTTTGGCCAGGGTCATTCCAGTC |
| | | TCTGGACCTGCCAGGTGTCTTAGCCAGTCCC |
| | | GAAACCTGCTGAAGACCACAGATGACATGGT |
| | | GAAGACGGCCAGAGAAAAACTGAAACATTAT |
| | | TCCTGCACTGCTGAAGACATCGACCATGAAG |
| | | ACATCACACGGGACCAAACCAGCACATTGAA |
| | | GACCTGTTTACCACTGGAACTACACAAGAACG |
| | | AGAGTTGCCTGGCTACTAGAGAGACTTCTTCC |
| | | ACAACAAGAGGGAGCTGCCTGCCCCCACAGA |
| | | AGACGTCTTTGATGATGACCCTGTGCCTTGGT |
| | | AGCATCTATGAGGACTTGAAGATGTACCAGA |
| | | CAGAGTTCCAGGCCATCAACGCAGCACTTCA |
| | | GAATCACAACCATCAGCAGATCATTCTAGACA |
| | | AGGGCATGCTGGTGGCCATCGACGAGCTGAT |
| | | GCAGTCTCTGAATCATAATGGCGAGACTCTGC |
| | | GCCAGAAACCTCCTGTGGGAGAAGCAGACCC |
| | | TTACAGAGTGAAAATGAAGCTCTGCATCCTGC |
| | | TTCACGCCTTCAGCACCCGCGTCGTGACCATC |
| | | AACAGGGTGATGGGCTATCTGAGTTCCGCCT |
| | | AAGTCGACTGAGTGACCGTGAAAGCTCACAA |
| | | AAAAAACTTCTCTAAATAGATTCGAGCACTAG |
| | | TATGTGGGTCCGGCAAGTACCCTGGTCATTCA |
| | | CTTGGGCTGTGCTGCAGTTGAGCTGGCAATC |
| | | AGGGTGGCTTCTAGAGGTCCCCAATGGGCCC |
| | | TGGAGGTCCCTCACCTTCTACCCAGCCTGGCT |
| | | CACAGTGTCAGAGGGAGCAAATGCCACCTTC |
| | | ACCTGCAGCTTGTCCAACTGGTCGGAGGATC |
| | | TTATGCTGAACTGGAACCGCCTGAGTCCCAG |
| | | CAACCAGACTGAAAAACAGGCCGCCTTCTGT |
| | | AATGGTTTGAGCCAACCCGTCCAGGATGCCC |
| | | GCTTCCAGATCATACAGCTGCCCAACAGGCAT |
| | | GACTTCCACATGAACATCCTTGACACACGGCG |
| | | CAATGACAGTGGCATCTACCTCTGTGGGGCC |
| | | ATCTCCCTGCACCCCAAGGCAAAAATCGAGG |
| | | AGAGCCCTGGAGCAGAGCTAGTGGTAACAGA |
| | | GAGAATCCTGGAGACCTCAACAAGATATCCC |
| | | AGCCCCTCGCCCAAACCAGAAGGCCGGTTTC |
| | | AAGGCTAGGAGCTCATAACTTCGTATAGCATA |
| | | CATTATACGAAGTTATTATTATATTTTTTATCTA |
| | | AAAAACTAAAAATAAACATTGATTAAATTTTAA |
| | | TATAATACTTAAAAATGGATGTTGTGTCGTTAGA |
| | | TAAACCGTTTATGTATTTTGAGGAAATTGATAAT |
| | | GAGTTAGATTACGAACCAGAAAGTGCAAATGAG |
| | | GTCGCAAAAAAACTGCCGTATCAAGGACAGTTA |
| | | AAACTATTACTAGGAGAATTATTTTTTCTTAGTA |
| | | AGTTACAGCGACACGGTATATTAGATGGTGCCA |
| | | CCGTAGTGTATATAGGATCTGCTCCCGGTACACA |
| | | TATACGTTATTTGAGAGATCATTTCTATAATTTA |
| | | GGAGTGATCATCAAATGGATGCTAATTGACGGC |
| | | CGCCATCATGATCCTATTTTAAATGGATTGCGTG |
| | | ATGTGACTCTAGTGACTCGGTTCGTTGATGAGGA |
| | | ATATCTACGATCCATCAAAAAACAACTGCATCCT |
| | | TCTAAGATTATTTTAATTTCTGATGTGAGATCCA |
| | | AACGAGGAGGAAATGAACCTAGTACGGCGGATT |
| | | TACTAAGTAATTACGCTCTACAAAATGTCATGAT |
| | | TAGTATTTTAAACCCCGTGGCGTCTAGTCTTAAA |
| | | TGGAGATGCCCGTTTCCAGATCAATGGATCAAG |
| | | GACTTTTATATCCCACACGGTAATAAAATGTTAC |
| | | AACCTTTTGCTCCTTCATATTCAGCTGAAATGAG |
| | | ATTATTAAGTATTTATACCGGTGAGAACATGAG |
| | | ACTGACTCGAGTTACCAAATCAGACGCTGTAAA |
| | | TTATGAAAAAAAGATGTACTACCTTAATAAGAT |
| | | CGTCCGTAACAAAGTAGTTGTTAACTTTGATTAT |
| | | CCTAATCAGGAATATGACTATTTTCACATGTACT |
| | | TTATGCTGAGGACCGTGTACTGCAATAAAACATT |
| | | TCCTACTACTAAAGCAAAGGTACTATTTCTACAA |
| | | CAATCTATATTTCGTTTCTTAAATATTCCAACAA |
| | | CATCAACTGAAAAAGTTAGTCATGAACCAATAC |
| | | AACG |

### Example 2: Construction of Human IL-12 and SPD-1 Expression System

A vaccinia virus, Western Reserve strain, is modified by replacing the gene encoding thymidine kinase (VACV094, J2R) with nucleic acids encoding a human IL-12 (hIL-12), a soluble human PD-1 (human sPD-1), and a fluorescent reporter. A plasmid transfer vector is generated comprising, in order, with no gaps, a 5′ recombination directing sequence (SEQ ID NO: 47), an SbfI cloning site (CCTGCAGG), a P7.5 promoter (SEQ ID NO: 44), a KpnI cloning site (GGTACC), open reading frame encoding human IL-12 (SEQ ID NO: 60), SalI cloning site followed by spacer (SEQ ID NO: 52), promoter P28 (SEQ ID NO: 41), SpeI cloning site (ACTAGT), open reading frame encoding human sPD-1 (SEQ ID NO: 63), a SacI cloning site (GAGCTC), a loxP sequence (SEQ ID NO: 54), a spacer sequence A (SEQ ID NO: 55), promoter 454 (SEQ ID NO: 42), fluorescent reporter protein (SEQ ID NO: 56), a PacI cloning site (TTAATTAA), spacer sequence B (SEQ ID NO: 57), a loxP sequence (SEQ ID NO: 54), and a 3′ recombination directing sequence (SEQ ID NO: 58). Following recombination and treatment with cre recombinase, the viral genome comprises the incorporated sequence of SEQ ID NO: 64. Sequences are shown in Table 7.

TABLE 7
Human IL-12 and human sPD-1 recombination sequences.

| SEQ ID | | |
|---|---|---|
| NO: | Description | Nucleic acid sequence |
| 60 | Human IL-12 | ATGTGTCACCAGCAGTTGGTCATCTCTTGGTTTT |
| | | CCCTGGTTTTTCTGGCATCTCCCCTCGTGGCCAT |
| | | ATGGGAACTGAAGAAAGATGTTTATGTCGTAGA |
| | | ATTGGATTGGTATCCGGATGCCCCTGGAGAAAT |
| | | GGTGGTCCTCACCTGTGACACCCCTGAAGAAGA |
| | | TGGTATCACCTGGACCTTGGACCAGAGCAGTGA |
| | | GGTCTTAGGCTCTGGCAAAACCCTGACCATCCA |
| | | AGTCAAAGAGTTTGGAGATGCTGGCCAGTACAC |
| | | CTGTCACAAAGGAGGCGAGGTTCTAAGCCATTC |
| | | GCTCCTGCTGCTTCACAAAAAGGAAGATGGAAT |
| | | TTGGTCCACTGATATTTTAAAGGACCAGAAAGA |
| | | ACCCAAAAATAAGACCTTTCTAAGATGCGAGGC |
| | | CAAGAATTATTCTGGACGTTTCACCTGCTGGTG |
| | | GCTGACGACAATCAGTACTGATTTGACATTCAG |
| | | TGTCAAAAGCAGCAGAGGCTCTTCTGACCCCCA |
| | | AGGGGTGACGTGCGGAGCTGCTACACTCTCTGC |
| | | AGAGAGAGTCAGAGGGGACAACAAGGAGTATG |
| | | AGTACTCAGTGGAGTGCCAGGAGGACAGTGCCT |
| | | GCCCAGCTGCTGAGGAGAGTCTGCCCATTGAGG |
| | | TCATGGTGGATGCCGTTCACAAGCTCAAGTATG |
| | | AAAACTACACCAGCAGCTTCTTCATCAGGGACA |
| | | TCATCAAACCTGACCCACCCAAGAACTTGCAGC |
| | | TGAAGCCATTAAAGAATTCTCGGCAGGTGGAGG |
| | | TCAGCTGGGAGTACCCTGACACCTGGAGTACTC |
| | | CACATTCCTACTTCTCCCTGACATTCTGCGTTCA |
| | | GGTCCAGGGCAAGAGCAAGAGAGAAAAGAAAG |
| | | ATAGAGTCTTCACGGACAAGACCTCAGCCACGG |
| | | TCATCTGCCGCAAAAATGCCAGCATTAGCGTGC |
| | | GGGCCCAGGACCGCTACTATAGCTCATCTTGGA |
| | | GCGAATGGGCATCTGTGCCCTGCAGTGGAGGTG |
| | | GCGGATCCGGCGGAGGTGGCTCAGGTGGCGGT |
| | | GGATCAGGAGGAGGTGGATCAGCTAGCTGGCC |
| | | CCCTGGGTCAGCCTCCCAGCCACCGCCCTCACC |
| | | TGCCGCGGCCACAGGTCTGCATCCAGCGGCTCG |
| | | CCCTGTGTCCCTGCAGTGCCGGCTCAGCATGTG |
| | | TCCAGCGCGCAGCCTCCTCCTTGTGGCTACCCT |
| | | GGTCCTCCTGGACCACCTCAGTTTGGCCAGAAA |
| | | CCTCCCCGTGGCCACTCCAGACCCAGGAATGTT |
| | | CCCATGCCTTCACCACTCCCAAAACCTGCTGAG |
| | | GGCCGTCAGCAACATGCTCCAGAAGGCCAGAC |
| | | AAACTCTAGAATTTTACCCTTGCACTTCTGAAG |
| | | AGATTGATCATGAAGATATCACAAAAGATAAA |
| | | ACCAGCACAGTGGAGGCCTGTTTACCATTGGAA |
| | | TTAACCAAGAATGAGAGTTGCCTAAATTCCAGA |
| | | GAGACCTCTTTCATAACTAATGGGAGTTGCCTG |
| | | GCCTCCAGAAAGACCTCTTTTATGATGGCCCTG |
| | | TGCCTTAGTAGTATTTATGAAGACTTGAAGATG |
| | | TACCAGGTGGAGTTCAAGACCATGAATGCAAA |
| | | GCTTCTGATGGATCCTAAGAGGCAGATCTTTCT |
| | | AGATCAAAACATGCTGGCAGTTATTGATGAGCT |
| | | GATGCAGGCCCTGAATTTCAACAGTGAGACTGT |
| | | GCCACAAAAATCCTCCCTTGAAGAACCGGATTT |
| | | TTATAAAACTAAAATCAAGCTCTGCATACTTCT |
| | | TCATGCTTTCAGAATTCGGGCAGTGACTATTGA |
| | | TAGAGTGATGAGCTATCTGAATGCTTCCTAA |
| 61 | human IL-12b | ATGTGTCACCAGCAGTTGGTCATCTCTTGGTTTT |
| | | CCCTGGTTTTTCTGGCATCTCCCCTCGTGGCCAT |
| | | ATGGGAACTGAAGAAAGATGTTTATGTCGTAGA |
| | | ATTGGATTGGTATCCGGATGCCCCTGGAGAAAT |
| | | GGTGGTCCTCACCTGTGACACCCCTGAAGAAGA |
| | | TGGTATCACCTGGACCTTGGACCAGAGCAGTGA |
| | | GGTCTTAGGCTCTGGCAAAACCCTGACCATCCA |
| | | AGTCAAAGAGTTTGGAGATGCTGGCCAGTACAC |
| | | CTGTCACAAAGGAGGCGAGGTTCTAAGCCATTC |
| | | GCTCCTGCTGCTTCACAAAAAGGAAGATGGAAT |
| | | TTGGTCCACTGATATTTTAAAGGACCAGAAAGA |
| | | ACCCAAAAATAAGACCTTTCTAAGATGCGAGGC |
| | | CAAGAATTATTCTGGACGTTTCACCTGCTGGTG |
| | | GCTGACGACAATCAGTACTGATTTGACATTCAG |
| | | TGTCAAAAGCAGCAGAGGCTCTTCTGACCCCCA |
| | | AGGGGTGACGTGCGGAGCTGCTACACTCTCTGC |
| | | AGAGAGAGTCAGAGGGGACAACAAGGAGTATG |
| | | AGTACTCAGTGGAGTGCCAGGAGGACAGTGCCT |
| | | GCCCAGCTGCTGAGGAGAGTCTGCCCATTGAGG |
| | | TCATGGTGGATGCCGTTCACAAGCTCAAGTATG |
| | | AAAACTACACCAGCAGCTTCTTCATCAGGGACA |
| | | TCATCAAACCTGACCCACCCAAGAACTTGCAGC |
| | | TGAAGCCATTAAAGAATTCTCGGCAGGTGGAGG |
| | | TCAGCTGGGAGTACCCTGACACCTGGAGTACTC |
| | | CACATTCCTACTTCTCCCTGACATTCTGCGTTCA |
| | | GGTCCAGGGCAAGAGCAAGAGAGAAAAGAAAG |
| | | ATAGAGTCTTCACGGACAAGACCTCAGCCACGG |
| | | TCATCTGCCGCAAAAATGCCAGCATTAGCGTGC |
| | | GGGCCCAGGACCGCTACTATAGCTCATCTTGGA |
| | | GCGAATGGGCATCTGTGCCCTGCAGT |
| | linker | SEQ ID NO: 50 |
| 62 | human IL-12a | TGGCCCCCTGGGTCAGCCTCCCAGCCACCGCCC |
| | | TCACCTGCCGCGGCCACAGGTCTGCATCCAGCG |
| | | GCTCGCCCTGTGTCCCTGCAGTGCCGGCTCAGC |
| | | ATGTGTCCAGCGCGCAGCCTCCTCCTTGTGGCT |
| | | ACCCTGGTCCTCCTGGACCACCTCAGTTTGGCC |
| | | AGAAACCTCCCCGTGGCCACTCCAGACCCAGGA |
| | | ATGTTCCCATGCCTTCACCACTCCCAAAACCTG |
| | | CTGAGGGCCGTCAGCAACATGCTCCAGAAGGCC |
| | | AGACAAACTCTAGAATTTTACCCTTGCACTTCT |
| | | GAAGAGATTGATCATGAAGATATCACAAAAGA |
| | | TAAAACCAGCACAGTGGAGGCCTGTTTACCATT |
| | | GGAATTAACCAAGAATGAGAGTTGCCTAAATTC |
| | | CAGAGAGACCTCTTTCATAACTAATGGGAGTTG |
| | | CCTGGCCTCCAGAAAGACCTCTTTTATGATGGC |
| | | CCTGTGCCTTAGTAGTATTTATGAAGACTTGAA |
| | | GATGTACCAGGTGGAGTTCAAGACCATGAATGC |
| | | AAAGCTTCTGATGGATCCTAAGAGGCAGATCTT |
| | | TCTAGATCAAAACATGCTGGCAGTTATTGATGA |
| | | GCTGATGCAGGCCCTGAATTTCAACAGTGAGAC |
| | | TGTGCCACAAAAATCCTCCCTTGAAGAACCGGA |
| | | TTTTTATAAAACTAAAATCAAGCTCTGCATACTT |
| | | CTTCATGCTTTCAGAATTCGGGCAGTGACTATT |
| | | GATAGAGTGATGAGCTATCTGAATGCTTCCTAA |
| 63 | N-terminal soluble human | ATGCAGATCCCACAGGCGCCCTGGCCAGTCGTC |
| | PD-1 | TGGGCGGTGCTACAACTGGGCTGGCGGCCAGG |
| | | ATGGTTCTTAGACTCCCCAGACAGGCCCTGGAA |
| | | CCCCCCCACCTTCTCCCCAGCCCTGCTCGTGGTG |
| | | ACCGAAGGGGACAACGCCACCTTCACCTGCAGC |
| | | TTCTCCAACACATCGGAGAGCTTCGTGCTAAAC |
| | | TGGTATCGCATGAGCCCCAGCAACCAGACGGAC |
| | | AAGCTGGCCGCCTTCCCCGAGGACCGCAGCCAG |
| | | CCCGGCCAGGACTGCCGCTTCCGTGTCACACAA |
| | | CTGCCCAACGGGCGTGACTTCCACATGAGCGTG |
| | | GTCAGGGCCCGGCGCAATGACAGCGGCACCTA |
| | | CCTCTGTGGGGCCATCTCCCTGGCCCCCAAGGC |
| | | GCAGATCAAAGAGAGCCTGCGGGCAGAACTCA |
| | | GGGTGACAGAGAGAAGGGCAGAAGTGCCCACA |
| | | GCCCACCCCAGCCCCTCACCCAGGCCAGCCGGC |
| | | CAGTTCCAAACCCTGGTGTAA |
| 64 | Final sequence in viral | ATTTACGGATTCACCAATAAAAATAAACTAGAG |
| | genome (non-native DNA | AAACTTAGTACTAATAAGGAACTAGAATCGTAT |
| | BOLD; transgene open | AGTTCTAGCCCTCTTCAAGAACCCATTAGGTTA |
| | reading frames | AATGATTTTCTGGGACTATTGGAATGTGTTAAA |
| | UNDERLINED) | AAGAATATTCCTCTAACAGATATTCCGACAAAG |
| | | GATTGATTACTATAAATGGAGAATGTTCCTAAT |
| | | GTATACTTTAATCCTGTGTTTATAGAGCCCACGT |
| | | TTAAACATTCTTTATTAAGTGTTTATAAACACAG |
| | | ATTAATAGTTTTATTTGAAGTATTCGTTGTATTC |
| | | ATTCTAATATATGTATTTTTTAGATCTGAATTAA |
| | | ATATGTTCTTCATGCCTAAACGAAAAATACCCG |
| | | ATCCTATTGATAGATTACGACGTGCTAATCTAG |
| | | CGTGTGAAGACGATAAATTAATGATCTATGGAT |
| | | TACCATGGATGACAACTCAAACATCTGCGTTAT |
| | | CAATAAATAGTAAACCGATAGTGTATAAAGATT |
| | | GTGCAAAGCTTTTGCGATCAATAAATGGATCAC |
| | | AACCAGTATCTCTTAACGATGTTCTTCGCAGAT |
| | | GATGATTCATTTTTTAAGTATTTGGCTAGTCAAG |
| | | ATGATGAATCTTCATTATCTGATATATTGCAAAT |
| | | CACTCAATATCTAGACTTTCTGTTATTATTATTG |
| | | ATCCAATCAAAAAATAAATTAGAAGCCGTGGGT |
| | | CATTGTTATGAATCTCTTTCAGAGGAATACAGA |
| | | CAATTGACAAAATTCACAGACTTTCAAGATTTT |
| | | AAAAAACTGTTTAACAAGGTCCCTATTGTTACA |
| | | GATGGAAGGGTCAAACTTAATAAAGGATATTTG |
| | | TTCGACTTTGTGATTAGTTTGATGCGATTCAAAA |
| | | AAGAATCCTCTCTAGCTACCACCGCAATAGATC |
| | | CTGTTAGATACATAGATCCTCGTCGCAATATCG |
| | | CATTTTCTAACGTGATGGATATATTAAAGTCGA |
| | | ATAAAGTGAACAATAATTAATTCTTTATTGTCA |
| | | TCCCTGCAGGTCACTAATTCCAAACCCACCC |
| | | GCTTTTTATAGTAAGTTTTTCACCCATAAATA |
| | | ATAAATACAATAATTAATTTCTCGTAAAAGTA |
| | | GAAAATATATTCTAATTTATTGCACGGTAAG |
| | | GAAGTAGATCATAAGGTACCATGTGTCACCA |
| | | GCAGTTGGTCATCTCTTGGTTTTCCCTGGTTT |
| | | TTCTGGCATCTCCCCTCGTGGCCATATGGGA |
| | | ACTGAAGAAAGATGTTTATGTCGTAGAATTG |
| | | GATTGGTATCCGGATGCCCCTGGAGAAATGG |
| | | TGGTCCTCACCTGTGACACCCCTGAAGAAGA |
| | | TGGTATCACCTGGACCTTGGACCAGAGCAGT |
| | | GAGGTCTTAGGCTCTGGCAAAACCCTGACCA |
| | | TCCAAGTCAAAGAGTTTGGAGATGCTGGCCA |
| | | GTACACCTGTCACAAAGGAGGCGAGGTTCTA |
| | | AGCCATTCGCTCCTGCTGCTTCACAAAAAGG |
| | | AAGATGGAATTTGGTCCACTGATATTTTAAA |
| | | GGACCAGAAAGAACCCAAAAATAAGACCTTT |
| | | CTAAGATGCGAGGCCAAGAATTATTCTGGAC |
| | | GTTTCACCTGCTGGTGGCTGACGACAATCAG |
| | | TACTGATTTGACATTCAGTGTCAAAAGCAGC |
| | | AGAGGCTCTTCTGACCCCCAAGGGGTGACGT |
| | | GCGGAGCTGCTACACTCTCTGCAGAGAGAGT |
| | | CAGAGGGGACAACAAGGAGTATGAGTACTCA |
| | | GTGGAGTGCCAGGAGGACAGTGCCTGCCCA |
| | | GCTGCTGAGGAGAGTCTGCCCATTGAGGTCA |
| | | TGGTGGATGCCGTTCACAAGCTCAAGTATGA |
| | | AAACTACACCAGCAGCTTCTTCATCAGGGAC |
| | | ATCATCAAACCTGACCCACCCAAGAACTTGC |
| | | AGCTGAAGCCATTAAAGAATTCTCGGCAGGT |
| | | GGAGGTCAGCTGGGAGTACCCTGACACCTGG |
| | | AGTACTCCACATTCCTACTTCTCCCTGACATT |
| | | CTGCGTTCAGGTCCAGGGCAAGAGCAAGAGA |
| | | GAAAAGAAAGATAGAGTCTTCACGGACAAGA |
| | | CCTCAGCCACGGTCATCTGCCGCAAAAATGC |
| | | CAGCATTAGCGTGCGGGCCCAGGACCGCTAC |
| | | TATAGCTCATCTTGGAGCGAATGGGCATCTG |
| | | TGCCCTGCAGTGGAGGTGGCGGATCCGGCG |
| | | GAGGTGGCTCAGGTGGCGGTGGATCAGGAG |
| | | GAGGTGGATCAGCTAGCTGGCCCCCTGGGTC |
| | | AGCCTCCCAGCCACCGCCCTCACCTGCCGCG |
| | | GCCACAGGTCTGCATCCAGCGGCTCGCCCTG |
| | | TGTCCCTGCAGTGCCGGCTCAGCATGTGTCC |
| | | AGCGCGCAGCCTCCTCCTTGTGGCTACCCTG |
| | | GTCCTCCTGGACCACCTCAGTTTGGCCAGAA |
| | | ACCTCCCCGTGGCCACTCCAGACCCAGGAAT |
| | | GTTCCCATGCCTTCACCACTCCCAAAACCTG |
| | | CTGAGGGCCGTCAGCAACATGCTCCAGAAGG |
| | | CCAGACAAACTCTAGAATTTTACCCTTGCACT |
| | | TCTGAAGAGATTGATCATGAAGATATCACAA |
| | | AAGATAAAACCAGCACAGTGGAGGCCTGTTT |
| | | ACCATTGGAATTAACCAAGAATGAGAGTTGC |
| | | CTAAATTCCAGAGAGACCTCTTTCATAACTAA |
| | | TGGGAGTTGCCTGGCCTCCAGAAAGACCTCT |
| | | TTTATGATGGCCCTGTGCCTTAGTAGTATTTA |
| | | TGAAGACTTGAAGATGTACCAGGTGGAGTTC |
| | | AAGACCATGAATGCAAAGCTTCTGATGGATC |
| | | CTAAGAGGCAGATCTTTCTAGATCAAAACAT |
| | | GCTGGCAGTTATTGATGAGCTGATGCAGGCC |
| | | CTGAATTTCAACAGTGAGACTGTGCCACAAA |
| | | AATCCTCCCTTGAAGAACCGGATTTTTATAAA |
| | | ACTAAAATCAAGCTCTGCATACTTCTTCATGC |
| | | TTTCAGAATTCGGGCAGTGACTATTGATAGA |
| | | GTGATGAGCTATCTGAATGCTTCCTAAGTCGA |
| | | CTGAGTGACCGTGAAAGCTCACAAAAAAAACTT |
| | | CTCTAAATAGATTCGAGCACTAGTATGCAGATC |
| | | CCACAGGCGCCCTGGCCAGTCGTCTGGGCG |
| | | GTGCTACAACTGGGCTGGCGGCCAGGATGGT |
| | | TCTTAGACTCCCCAGACAGGCCCTGGAACCC |
| | | CCCCACCTTCTCCCCAGCCCTGCTCGTGGTG |
| | | ACCGAAGGGGACAACGCCACCTTCACCTGCA |
| | | GCTTCTCCAACACATCGGAGAGCTTCGTGCT |
| | | AAACTGGTATCGCATGAGCCCCAGCAACCAG |
| | | ACGGACAAGCTGGCCGCCTTCCCCGAGGACC |
| | | GCAGCCAGCCCGGCCAGGACTGCCGCTTCCG |
| | | TGTCACACAACTGCCCAACGGGCGTGACTTC |
| | | CACATGAGCGTGGTCAGGGCCCGGCGCAAT |
| | | GACAGCGGCACCTACCTCTGTGGGGCCATCT |
| | | CCCTGGCCCCCAAGGCGCAGATCAAAGAGAG |
| | | CCTGCGGGCAGAACTCAGGGTGACAGAGAG |
| | | AAGGGCAGAAGTGCCCACAGCCCACCCCAGC |
| | | CCCTCACCCAGGCCAGCCGGCCAGTTCCAAA |
| | | CCCTGGTGTAAGAGCTCATAACTTCGTATAG |
| | | CATACATTATACGAAGTTATTATTATATTTTTT |
| | | ATCTAAAAAACTAAAAATAAACATTGATTAAAT |
| | | TTTAATATAATACTTAAAAATGGATGTTGTGTC |
| | | GTTAGATAAACCGTTTATGTATTTTGAGGAAAT |
| | | TGATAATGAGTTAGATTACGAACCAGAAAGTGC |
| | | AAATGAGGTCGCAAAAAAACTGCCGTATCAAG |
| | | GACAGTTAAAACTATTACTAGGAGAATTATTTT |
| | | TTCTTAGTAAGTTACAGCGACACGGTATATTAG |
| | | ATGGTGCCACCGTAGTGTATATAGGATCTGCTC |
| | | CCGGTACACATATACGTTATTTGAGAGATCATT |
| | | TCTATAATTTAGGAGTGATCATCAAATGGATGC |
| | | TAATTGACGGCCGCCATCATGATCCTATTTTAA |
| | | ATGGATTGCGTGATGTGACTCTAGTGACTCGGT |
| | | TCGTTGATGAGGAATATCTACGATCCATCAAAA |
| | | AACAACTGCATCCTTCTAAGATTATTTTAATTTC |
| | | TGATGTGAGATCCAAACGAGGAGGAAATGAAC |
| | | CTAGTACGGCGGATTTACTAAGTAATTACGCTC |
| | | TACAAAATGTCATGATTAGTATTTTAAACCCCG |
| | | TGGCGTCTAGTCTTAAATGGAGATGCCCGTTTC |
| | | CAGATCAATGGATCAAGGACTTTTATATCCCAC |
| | | ACGGTAATAAAATGTTACAACCTTTTGCTCCTTC |
| | | ATATTCAGCTGAAATGAGATTATTAAGTATTTA |
| | | TACCGGTGAGAACATGAGACTGACTCGAGTTAC |
| | | CAAATCAGACGCTGTAAATTATGAAAAAAAGA |
| | | TGTACTACCTTAATAAGATCGTCCGTAACAAAG |
| | | TAGTTGTTAACTTTGATTATCCTAATCAGGAATA |
| | | TGACTATTTTCACATGTACTTTATGCTGAGGACC |
| | | GTGTACTGCAATAAAACATTTCCTACTACTAAA |
| | | GCAAAGGTACTATTTCTACAACAATCTATATTT |
| | | CGTTTCTTAAATATTCCAACAACATCAACTGAA |
| | | AAAGTTAGTCATGAACCAATACAACG |

### Example 3: Measurement of Tumor Size Following Treatment with Virus Expressing SPD-1 and IL-12

Renca cells were implanted into the flank of Balb/c mice. After 12 days mice were sorted such that each treatment group had tumors 50-100 mm³. Mice were treated with 1E7 PFU virus via intratumoral injection. Tumor size was measured after 45 days.

B16F10 cells were mixed 1:1 with Matrigel and implanted into the flank of C57/Black 6 mice. After 5 days mice were sorted such that each treatment group had tumors 50-100 mm³. Mice were treated with 1E7 PFU virus via intratumoral injection. Tumor size was measured after 38 days.

Mice with induced with tumors were divided into the following treatment groups: buffer control, TK-vaccinia virus, and TK-vaccinia virus expressing IL-12 and murine sPD-1 as described in Example 1. Average tumor volume was measured in each treatment group.

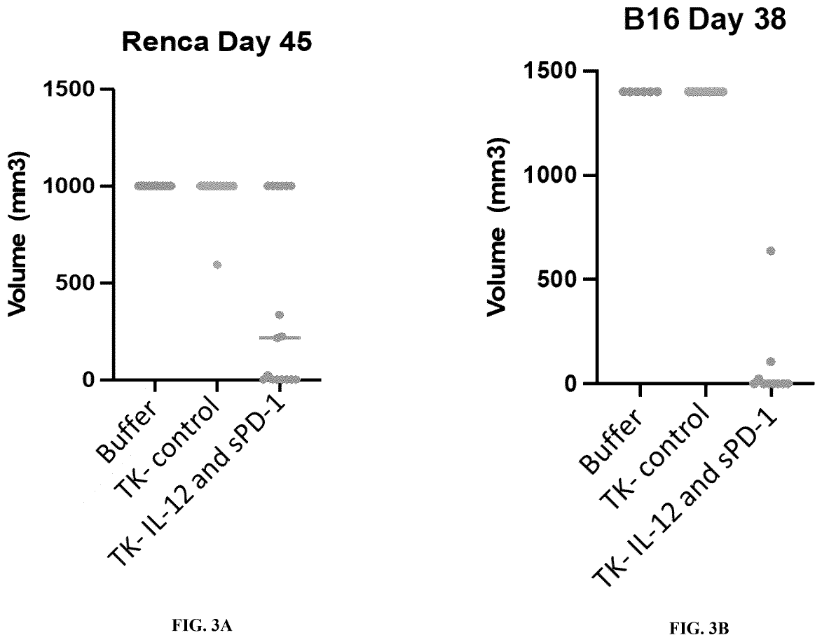

Tumor volumes of Renca tumor groups are shown in FIG. 3A. Mice sacrificed due to tumor burden prior to final measurement appear as 1000 mm³ in FIG. 3A. Mice with induced Renca tumors treated with virus expressing the combination IL-12 and murine sPD-1 showed an average tumor volume of about 200 mm³ and a Complete Response (CR) of 40% 45 days post treatment. In comparison, control groups treated with buffer control or TK-virus had an average tumor volume of about at least 1000 mm³.

Tumor volumes of B16 tumor groups are shown in FIG. 3B. Mice sacrificed due to tumor burden prior to final measurement appear as 1400 mm³ in FIG. 3B. Mice with induced B16 tumors treated with virus expressing IL-12 and murine sPD-1 had an average tumor volume below detection levels and a CR of 90% 38 days post treatment. In comparison, control groups treated with buffer control or TK-virus had an average tumor volume of at least 1400 mm³.

### Example 4: Deletion of B8r Gene (IFNg Binding Protein)

Interferon gamma (IFNg) plays a role in anticancer immunity by promoting activity of various immune cells. IL-12 and sPD-1 are expected to require IFNg production for activity. The vaccinia IFNg binding protein, expressed by the B8R gene, acts as a secreted decoy receptor, removing extracellular IFNg. Virus as described in Example 1 was further modified to remove the B8R gene.

A transfer vector was generated comprising, in order, a reporter vector 5′ recombination directing sequence (SEQ ID NO: 65), a spacer sequence C (SEQ ID NO: 66), a 454 promoter (SEQ ID NO: 42), a fluorescent reporter protein (SEQ ID NO: 67), a reporter vector 3′ recombination directing sequence (SEQ ID NO: 68). Components of the vector replaced the B8R open reading frame with a nucleic acid expressing fluorescent reporter gene for plaque selection purposes. Selected plaques were then treated with a second transfer vector to remove the promoter and fluorescent reporter gene, comprising the reporter vector 5′ recombination directing sequence (SEQ ID NO: 65) and reporter vector 3′ recombination directing sequence (SEQ ID NO: 68). The final sequence of the modified B8R locus is as in SEQ ID NO: 69. The original location of the B8R open reading frame is designated by paired brackets [ ]. Sequences are shown in Table 8.

TABLE 8
B8R removal vector sequences

| SEQ | | |
|---|---|---|
| ID NO: | Description | Nucleic acid sequence |
| 65 | Reporter vector, 5′ | GTACGTACTATGCCAAATGAATCACGTGTTAAA |
| | recombination-directing | CAATGGGTGATGGATACACTTAATGGTATAATG |
| | sequence | ATGAATGAACGCGATGTTTCTGTAAGCGTTGGC |
| | | ACCGGAATACTATTCATGGAAATGTTTTTCGATT |
| | | ACAATAAAAATAGTATCAACAATCAACTAATGT |
| | | ATGATATAATTAATAGCGTATCTATAATTCTAGC |
| | | TAATGAGAGATATAGAAGCGCTTTTAACGACGA |
| | | TGGTATATACATCCGTAGAAATATGATTAACAA |
| | | GTTGTACGGATACGCATCTCTAACTACTATTGGC |
| | | ACGATCGCTGGAGGTGTTTGTTATTATCTGTTGA |
| | | TGCATCTAGTTAGTTTGTATAAATAATTATTTCA |
| | | ATATACTAGTTAAAATTTTAAGATTTTAAATGTA |
| | | TAAAAAACTAATAACGTTTTTATTTGTAATAGGT |
| | | GCATTAGCATCCTATTCGAATAATGAGTACACTC |
| | | CGTTTAATAAACTGAGTGTAAAACTCTATATAGA |
| | | TGGAGTAGATAATATAGAAAATTCATATACTGA |
| | | TGATAATAATGAATTGGTGTTAAATTTTAAAGAG |
| | | TACACAATTTCTATTATTACAGAGTCATGCGACG |
| | | TCGGATTTGATTCCATAGATATAGATGTTATAAA |
| | | CGACTATAAAATTATTGATATGTATACCATTGAC |
| | | TCGTCTACTATTCAACGCAGAGGTCACACGTGTA |
| | | GAATATCTACCAAATTATCATGCCATTATGATAA |
| | | GTACCCTTATATTCACAAATATGATGGTGATGAG |
| | | CGACAATATTCTATTACTGCAGAGGGAAAATGC |
| | | TATAAAGGAATAAAATATGAAATAAGTATGATC |
| | | AACGATGATACTCTATTGAGAAAACATACTCTTA |
| | | AAATTGGATCTACTTATATATTTGATCGTCATGG |
| | | ACATAGTAATACATATTATTCAAAATATGATTTT |
| | | TAAAAATTTAAAATATATTATCACTTCAGTGACA |
| | | GTAGTCAAATAACAAACAACACC |
| 66 | Spacer sequence C | CGAAGTTATAATTCTGCAGA |
| 67 | fluorescent reporter protein | ATGCGTAAAGGCGAAGAGCTGTTCACTGGTGTC |
| | | GTCCCTATTCTGGTGGAACTGGATGGTGATGTCA |
| | | ACGGTCATAAGTTTTCCGTGCGTGGCGAGGGTG |
| | | AAGGTGACGCAACTAATGGTAAACTGACGCTGA |
| | | AGTTCATCTGTACTACTGGTAAACTGCCGGTCCC |
| | | TTGGCCGACTCTGGTAACGACGCTGACTTATGGT |
| | | GTTCAGTGCTTTGCTCGTTATCCGGACCATATGA |
| | | AGCAGCATGACTTCTTCAAGTCCGCCATGCCGG |
| | | AAGGCTATGTGCAGGAACGCACGATTTCCTTTA |
| | | AGGATGACGGCACGTACAAAACGCGTGCGGAAG |
| | | TGAAATTTGAAGGCGATACCCTGGTAAACCGCA |
| | | TTGAGCTGAAAGGCATTGACTTTAAAGAAGACG |
| | | GCAATATCCTGGGCCATAAGCTGGAATACAATT |
| | | TTAACAGCCACAATGTTTACATCACCGCCGATAA |
| | | ACAAAAAAATGGCATTAAAGCGAATTTTAAAAT |
| | | TCGCCACAACGTGGAGGATGGCAGCGTGCAGCT |
| | | GGCTGATCACTACCAGCAAAACACTCCAATCGG |
| | | TGATGGTCCTGTTCTGCTGCCAGACAATCACTAT |
| | | CTGAGCACGCAAAGCGTTCTGTCTAAAGATCCG |
| | | AACGAGAAACGCGATCATATGGTTCTGCTGGAG |
| | | TTCGTAACCGCAGCGGGCATCACGCATGGTATG |
| | | GATGAACTGTACAAATAG |
| 68 | Reporter vector, 3′ | CTAATTTTTATTAATGATACAAAAACGAAATAA |
| | recombination-directing | AACTGCATATTATACACTGGTTAACGCCCTTATA |
| | sequence | GGCTCTAACCATTTTCAAGATGAGGTCCCTGATT |
| | | ATAGTCCTTCTGTTCCCCTCTATCATCTACTCCAT |
| | | GTCTATTAGACAATGTGAGAAGACTGAAGAGGA |
| | | AACATGGGGATTGAAAATAGGGTTGTGTATAAT |
| | | TGCCAAAGATTTCTATCCCGAAAGAACTGATTGC |
| | | AGTGTTCATCTCCCAACTGCAAGTGAAGGATTG |
| | | ATAACTGAAGGCAATGGATTCAGGGATATACGA |
| | | AACACCGATAAATTATAAAAAAAGCAATGTGTC |
| | | CGCTGTTTCCGTTAATAATACTATTTTCGTAACT |
| | | GGCGGATTATTCATAAATAACTCTAATAGCACG |
| | | ATCGTGGTTAACAATATGGAAAAACTTGACATTT |
| | | ATAAAGACAAACAATGGTCGATTATAGAAATGC |
| | | CTATGGCTAGGGTATATCACGGCATCGACTCGA |
| | | CATTTGGAATGTTATATTTTGCCGGAGGTCTATC |
| | | CGTTACCGAACAATATGGTAATTTAGAGAAAAA |
| | | CAACGAGATATCTTGTTACAATCCTAGAACGAA |
| | | TAAGTGGTTTGATATTTCATATACTATTTATAAG |
| | | ATATCCATATCATCATTGTGTAAACTAAATAACG |
| | | TCTTCTATGTATTTAGTAAGGACATTGGATATGT |
| | | GGAAAAGTATGATGGTGCATGGAAGTTAGTACA |
| | | TGATCGTCTCCCCGCTATAAAGGCATTATCAACT |
| | | TCTCCTTATTGATTGAAAATGAAAATATAAATAG |
| | | TTTTTATGTATAGCAGTATTACCCTATAGTTTTAT |
| | | TGCTTACTACTAACATGGATACAGATG |
| 69 | The sequence of the modified | GTACGTACTATGCCAAATGAATCACGTGTTAAA |
| | (deleted) B8R locus is | CAATGGGTGATGGATACACTTAATGGTATAATG |
| | (original location of B8R open | ATGAATGAACGCGATGTTTCTGTAAGCGTTGGC |
| | reading frame is designated by | ACCGGAATACTATTCATGGAAATGTTTTTCGATT |
| | paired brackets []) | ACAATAAAAATAGTATCAACAATCAACTAATGT |
| | | ATGATATAATTAATAGCGTATCTATAATTCTAGC |
| | | TAATGAGAGATATAGAAGCGCTTTTAACGACGA |
| | | TGGTATATACATCCGTAGAAATATGATTAACAA |
| | | GTTGTACGGATACGCATCTCTAACTACTATTGGC |
| | | ACGATCGCTGGAGGTGTTTGTTATTATCTGTTGA |
| | | TGCATCTAGTTAGTTTGTATAAATAATTATTTCA |
| | | ATATACTAGTTAAAATTTTAAGATTTTAAATGTA |
| | | TAAAAAACTAATAACGTTTTTATTTGTAATAGGT |
| | | GCATTAGCATCCTATTCGAATAATGAGTACACTC |
| | | CGTTTAATAAACTGAGTGTAAAACTCTATATAGA |
| | | TGGAGTAGATAATATAGAAAATTCATATACTGA |
| | | TGATAATAATGAATTGGTGTTAAATTTTAAAGAG |
| | | TACACAATTTCTATTATTACAGAGTCATGCGACG |
| | | TCGGATTTGATTCCATAGATATAGATGTTATAAA |
| | | CGACTATAAAATTATTGATATGTATACCATTGAC |
| | | TCGTCTACTATTCAACGCAGAGGTCACACGTGTA |
| | | GAATATCTACCAAATTATCATGCCATTATGATAA |
| | | GTACCCTTATATTCACAAATATGATGGTGATGAG |
| | | CGACAATATTCTATTACTGCAGAGGGAAAATGC |
| | | TATAAAGGAATAAAATATGAAATAAGTATGATC |
| | | AACGATGATACTCTATTGAGAAAACATACTCTTA |
| | | AAATTGGATCTACTTATATATTTGATCGTCATGG |
| | | ACATAGTAATACATATTATTCAAAATATGATTTT |
| | | TAAAAATTTAAAATATATTATCACTTCAGTGACA |
| | | GTAGTCAAATAACAAACAACACC[]CTAATTTTTA |
| | | TTAATGATACAAAAACGAAATAAAACTGCATAT |
| | | TATACACTGGTTAACGCCCTTATAGGCTCTAACC |
| | | ATTTTCAAGATGAGGTCCCTGATTATAGTCCTTC |
| | | TGTTCCCCTCTATCATCTACTCCATGTCTATTAGA |
| | | CAATGTGAGAAGACTGAAGAGGAAACATGGGG |
| | | ATTGAAAATAGGGTTGTGTATAATTGCCAAAGA |
| | | TTTCTATCCCGAAAGAACTGATTGCAGTGTTCAT |
| | | CTCCCAACTGCAAGTGAAGGATTGATAACTGAA |
| | | GGCAATGGATTCAGGGATATACGAAACACCGAT |
| | | AAATTATAAAAAAAGCAATGTGTCCGCTGTTTCC |
| | | GTTAATAATACTATTTTCGTAACTGGCGGATTAT |
| | | TCATAAATAACTCTAATAGCACGATCGTGGTTAA |
| | | CAATATGGAAAAACTTGACATTTATAAAGACAA |
| | | ACAATGGTCGATTATAGAAATGCCTATGGCTAG |
| | | GGTATATCACGGCATCGACTCGACATTTGGAAT |
| | | GTTATATTTTGCCGGAGGTCTATCCGTTACCGAA |
| | | CAATATGGTAATTTAGAGAAAAACAACGAGATA |
| | | TCTTGTTACAATCCTAGAACGAATAAGTGGTTTG |
| | | ATATTTCATATACTATTTATAAGATATCCATATC |
| | | ATCATTGTGTAAACTAAATAACGTCTTCTATGTA |
| | | TTTAGTAAGGACATTGGATATGTGGAAAAGTAT |
| | | GATGGTGCATGGAAGTTAGTACATGATCGTCTCC |
| | | CCGCTATAAAGGCATTATCAACTTCTCCTTATTG |
| | | ATTGAAAATGAAAATATAAATAGTTTTTATGTAT |
| | | AGCAGTATTACCCTATAGTTTTATTGCTTACTAC |
| | | TAACATGGATACAGATG |

### Example 5: Measurement of Tumor Size Following Treatment with B8R-Virus Expressing SPD-1 and IL-12

Lewis lung carcinoma (LLC) cells were implanted into the flank of C57/Black mice. After 5 days mice were sorted such that each treatment group had tumors 50-100 mm³. Mice were treated with 1E7 PFU virus via intratumoral injection. Tumor size was measured after 31 days.

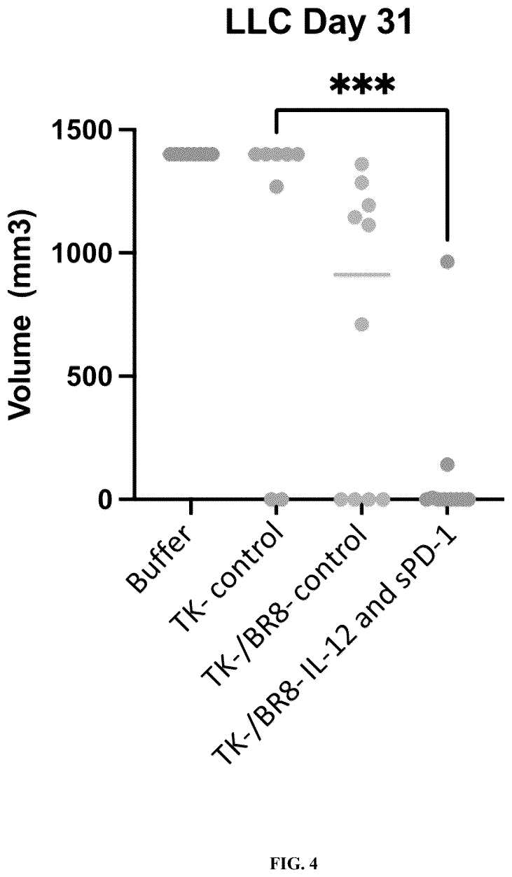

Mice with induced with tumors were divided into the following treatment groups: buffer control, TK-vaccinia virus, TK-/B8R-vaccinia virus, and TK-/B8R-vaccinia virus expressing IL-12 and murine sPD-1 as described in Example 4. Average tumor volume was measured in each treatment group. Measurements at day 31 are shown in FIG. 4. Mice sacrificed due to tumor burden prior to final measurement appear as 1400 mm³ in figure.

Mice with treated buffer control or TK-vaccinia showed an average tumor volume of at least 1400 mm³ after 31 days. Mice treated with TK-/B8R-vaccinia virus had an average tumor volume of about 900 mm³. Mice treated with TK-/B8R-vaccinia virus expressing IL-12 and murine sPD-1 had an average tumor volume below detectable levels and a CR of 80% after 31 days.

### Example 5: Addition of Murine CXCR3 Expression System

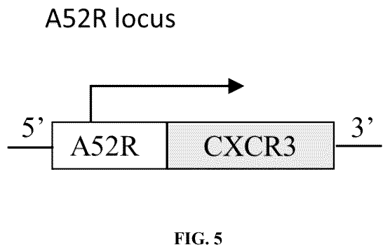

A vaccinia virus of any of the previous examples is modified by replacing the gene encoding A52 with nucleic acid encoding murine CXCR3 and a fluorescent reporter. A plasmid transfer vector is generated comprising, in order, with no gaps, an upstream recombination directing sequence B (SEQ ID NO: 70), an open reading frame encoding murine CXCR3 (SEQ ID NO: 71), a stop codon, SacI cloning site, and short spacer (SEQ ID NO: 72), a loxP site (SEQ ID NO: 54), a spacer followed by viral promoter driving expression of GFP-pac reporter (SEQ ID NO: 73), PacI cloning site (TTAATTAA), a short spacer B (SEQ ID NO: 57), and a downstream recombination directing sequence B (SEQ ID NO: 74). Following recombination and treatment with cre recombinase, the viral genome comprises the incorporated sequence of SEQ ID NO: 75. Sequences are shown in Table 9. A schematic representation of the promoter and transgene inserted at an A52R gene is shown in FIG. 5.

TABLE 9
Murine CXCR3 recombination sequences.

| SEQ | | |
|---|---|---|
| ID NO: | Description | Nucleic acid sequence |
| 70 | Upstream recombination | CTCATCAGACATATCGTGTGCATGCGTGGAGTTA |
| | directing sequence B | AATTCATATCACGTAGCCATTCTTCCCATGGATGT |
| | | TTCCTTTTTTACCAAAGGAAATGCATCATTGATTA |
| | | TTCTCCTGTTTGATTTCTCTATCGATGCAGCACCT |
| | | CTCTTAAGAAGTGTAACCGATAATAATGTTATTA |
| | | TATCTAGACACCAGCGTCTACATGACGAGCTTCC |
| | | GAGTTCCAATTGGTTCAAGTTTTACATAAGTATA |
| | | AAGTCCGACTATTGTTCTATATTATATATGGTTGT |
| | | TGATGGATCTGTGATGCATGCGATAGCTGATAAT |
| | | AGAACTCACGCAATTATTAGCAAAAATATATTAG |
| | | ACAATACTACGATTAACGATGAGTGTAGATGCTG |
| | | TTATTTTGAACCACAGATTAGGATTCTTGATAGA |
| | | GATGAGATGCTCAATGGATCATCGTGTGATATGA |
| | | ACAGACATTGTATTATGATGAATTTACCTGATGT |
| | | AGGCAAATTTGGATCTAGTATGTTGGGGAAATAT |
| | | GAACCTGACATGATTAAGATTGCTCTTTCGGTGG |
| | | CTGGTAATTTAATAAGAAATCGAGACTACATTCC |
| | | CGGGAGACGAGGATATAGCTACTACGTTTACGGT |
| | | ATAGCCTCTAGATAATTTTTTTAAGCACGAAATA |
| | | AAAAACATAATTTTAAACCAATCTATTTCATACT |
| | | ATTTTGTGTGATCACC |
| 71 | Open reading frame encoding | ATGTACCTTGAGGTTAGTGAACGTCAAGTGCTAG |
| | murine CXCR3 | ATGCCTCGGACTTTGCCTTTCTTCTGGAAAACAGC |
| | | ACCTCTCCCTACGATTATGGGGAAAACGAGAGCG |
| | | ACTTCTCTGACTCCCCGCCCTGCCCACAGGATTTC |
| | | AGCCTGAACTTTGACAGAACCTTCCTGCCAGCCC |
| | | TCTACAGCCTCCTCTTCTTGCTGGGGCTGCTAGGC |
| | | AATGGGGCGGTGGCTGCTGTGCTACTGAGTCAGC |
| | | GCACTGCCCTGAGCAGCACGGACACCTTCCTGCT |
| | | CCACCTGGCTGTAGCCGATGTTCTGCTGGTGTTAA |
| | | CTCTTCCATTGTGGGCAGTGGATGCTGCTGTCCAG |
| | | TGGGTTTTCGGCCCTGGCCTCTGCAAAGTGGCAG |
| | | GCGCCTTGTTCAACATCAACTTCTATGCAGGGGC |
| | | CTTCCTGCTGGCTTGTATAAGCTTCGACAGATATC |
| | | TGAGCATAGTGCACGCCACCCAGATCTACCGCAG |
| | | GGACCCCCGGGTACGTGTAGCCCTCACCTGCATA |
| | | GTTGTATGGGGTCTCTGTCTGCTCTTTGCCCTCCC |
| | | AGATTTCATCTACCTATCAGCCAACTACGATCAG |
| | | CGCCTCAATGCCACCCATTGCCAGTACAACTTCC |
| | | CACAGGTGGGTCGCACTGCTCTGCGTGTACTGCA |
| | | GCTAGTGGCTGGTTTCCTGCTGCCCCTTCTGGTCA |
| | | TGGCCTACTGCTATGCCCATATCCTAGCTGTTCTG |
| | | CTGGTCTCCAGAGGCCAGAGGCGTTTTCGAGCTA |
| | | TGAGGCTAGTGGTAGTGGTGGTGGCAGCCTTTGC |
| | | TGTCTGCTGGACCCCCTATCACCTGGTGGTGCTAG |
| | | TGGATATCCTCATGGATGTGGGAGTTTTGGCCCG |
| | | CAACTGTGGTCGAGAAAGCCACGTGGATGTGGCC |
| | | AAGTCAGTCACCTCGGGCATGGGGTACATGCACT |
| | | GCTGCCTCAATCCGCTGCTCTATGCCTTTGTGGGA |
| | | GTGAAGTTCAGAGAGCAAATGTGGATGTTGTTCA |
| | | CGCGCCTGGGCCGCTCTGACCAGAGAGGGCCCCA |
| | | GCGGCAGCCGTCATCTTCACGGAGAGAATCATCC |
| | | TGGTCTGAGACAACTGAGGCCTCCTACCTGGGCT |
| | | TGTAA |
| 72 | Stop codon, SacI cloning site, | TAGGAGCTCTAGA |
| | and short spacer | |
| 73 | Spacer followed by viral | AATTCTGCAGATGGATCATAAAAATATAGTAGAA |
| | promoter driving expression of | TTTCATTTTGTTTTTTTCTATGCTATAAATAGCTTC |
| | GFP-pac reporter | TCGAGCATGCGTAAAGGCGAAGAGCTGTTCACTG |
| | | GTGTCGTCCCTATTCTGGTGGAACTGGATGGTGA |
| | | TGTCAACGGTCATAAGTTTTCCGTGCGTGGCGAG |
| | | GGTGAAGGTGACGCAACTAATGGTAAACTGACGC |
| | | TGAAGTTCATCTGTACTACTGGTAAACTGCCGGT |
| | | CCCTTGGCCGACTCTGGTAACGACGCTGACTTAT |
| | | GGTGTTCAGTGCTTTGCTCGTTATCCGGACCATAT |
| | | GAAGCAGCATGACTTCTTCAAGTCCGCCATGCCG |
| | | GAAGGCTATGTGCAGGAACGCACGATTTCCTTTA |
| | | AGGATGACGGCACGTACAAAACGCGTGCGGAAG |
| | | TGAAATTTGAAGGCGATACCCTGGTAAACCGCAT |
| | | TGAGCTGAAAGGCATTGACTTTAAAGAAGACGGC |
| | | AATATCCTGGGCCATAAGCTGGAATACAATTTTA |
| | | ACAGCCACAATGTTTACATCACCGCCGATAAACA |
| | | AAAAAATGGCATTAAAGCGAATTTTAAAATTCGC |
| | | CACAACGTGGAGGATGGCAGCGTGCAGCTGGCTG |
| | | ATCACTACCAGCAAAACACTCCAATCGGTGATGG |
| | | TCCTGTTCTGCTGCCAGACAATCACTATCTGAGCA |
| | | CGCAAAGCGTTCTGTCTAAAGATCCGAACGAGAA |
| | | ACGCGATCATATGGTTCTGCTGGAGTTCGTAACC |
| | | GCAGCGGGCATCACGCATGGTATGGATGAACTGT |
| | | ACAAAGGAGGCTCAGGAACCGAGTACAAGCCCA |
| | | CGGTGCGCCTCGCCACCCGCGACGACGTCCCCCG |
| | | GGCCGTACGCACCCTCGCCGCCGCGTTCGCCGAC |
| | | TACCCCGCCACGCGCCACACCGTCGACCCGGACC |
| | | GCCACATCGAGCGGGTCACCGAGCTGCAAGAACT |
| | | CTTCCTCACGCGCGTCGGGCTCGACATCGGCAAG |
| | | GTGTGGGTCGCGGACGACGGCGCCGCGGTGGCG |
| | | GTCTGGACCACGCCGGAGAGCGTCGAAGCGGGG |
| | | GCGGTGTTCGCCGAGATCGGCCCGCGCATGGCCG |
| | | AGTTGAGCGGTTCCCGGCTGGCCGCGCAGCAACA |
| | | GATGGAAGGCCTCCTGGCGCCGCACCGGCCCAAG |
| | | GAGCCCGCGTGGTTCCTGGCCACCGTCGGCGTCT |
| | | CGCCCGACCACCAGGGCAAGGGTCTGGGCAGCG |
| | | CCGTCGTGCTCCCCGGAGTGGAGGCGGCCGAGCG |
| | | CGCCGGGGTGCCCGCCTTCCTGGAGACCTCCGCG |
| | | CCCCGCAACCTCCCCTTCTACGAGCGGCTCGGCTT |
| | | CACCGTCACCGCCGACGTCGAGTGCCCGAAGGAC |
| | | CGCGCGACCTGGTGCATGACCCGCAAGCCCGGTG |
| | | CCTGA |
| | PacI cloning site | TTAATTAA |
| 74 | Downstream recombination | AAAGTTAAAAGTTAATGAGAGCAAAAATATATA |
| | directing sequence B | AGGTTGTATTCCATATTTGTTATTTTTTCTGTAAT |
| | | AGTTAGAAAAATACATTCGATGGTCTATCTACCA |
| | | GATTATTATGTGTTATAAGGTACTTTTTCTCATAA |
| | | TAAACTAGAGTATGAGTAAGATAGTGTTTTTCAA |
| | | AACATATAAATCTAAAATTGATGGATGAGATATA |
| | | CAGCTATTAATTTCGAAAATATATTTTAATCTGAT |
| | | AACTTTAAACATGGATTTTTGATGGTGGTTTAACG |
| | | TTTTAAAAAAAGATTTTGTTATTGTAGTATATGAT |
| | | AATATTAAAAGATGGATATAAAGAATTTGCTGAC |
| | | TGCATGTACTATTTTTTACATTACTACATTGGCTA |
| | | CGGCAGATATACCTACTCCGCCACCAACGGGTCA |
| | | TGTGACAAGGGAGAATATCTTGATAAGAGGCATA |
| | | ATCAATGTTGTAATCGGTGTCCACCTGGAGAATT |
| | | TGCCAAGGTTAGATGTAAT |
| 75 | Sequence incorporated in viral | CTCATCAGACATATCGTGTGCATGCGTGGAGTTA |
| | genome (non-native DNA | AATTCATATCACGTAGCCATTCTTCCCATGGATGT |
| | BOLD; CXCR3 transgene | TTCCTTTTTTACCAAAGGAAATGCATCATTGATTA |
| | UNDERLINED) | TTCTCCTGTTTGATTTCTCTATCGATGCAGCACCT |
| | | CTCTTAAGAAGTGTAACCGATAATAATGTTATTA |
| | | TATCTAGACACCAGCGTCTACATGACGAGCTTCC |
| | | GAGTTCCAATTGGTTCAAGTTTTACATAAGTATA |
| | | AAGTCCGACTATTGTTCTATATTATATATGGTTGT |
| | | TGATGGATCTGTGATGCATGCGATAGCTGATAAT |
| | | AGAACTCACGCAATTATTAGCAAAAATATATTAG |
| | | ACAATACTACGATTAACGATGAGTGTAGATGCTG |
| | | TTATTTTGAACCACAGATTAGGATTCTTGATAGA |
| | | GATGAGATGCTCAATGGATCATCGTGTGATATGA |
| | | ACAGACATTGTATTATGATGAATTTACCTGATGT |
| | | AGGCAAATTTGGATCTAGTATGTTGGGGAAATAT |
| | | GAACCTGACATGATTAAGATTGCTCTTTCGGTGG |
| | | CTGGTAATTTAATAAGAAATCGAGACTACATTCC |
| | | CGGGAGACGAGGATATAGCTACTACGTTTACGGT |
| | | ATAGCCTCTAGATAATTTTTTTAAGCACGAAATA |
| | | AAAAACATAATTTTAAACCAATCTATTTCATACT |
| | | ATTTTGTGTGATCACCATGTACCTTGAGGTTAGT |
| | | GAACGTCAAGTGCTAGATGCCTCGGACTTTGC |
| | | CTTTCTTCTGGAAAACAGCACCTCTCCCTACGA |
| | | TTATGGGGAAAACGAGAGCGACTTCTCTGACT |
| | | CCCCGCCCTGCCCACAGGATTTCAGCCTGAAC |
| | | TTTGACAGAACCTTCCTGCCAGCCCTCTACAG |
| | | CCTCCTCTTCTTGCTGGGGCTGCTAGGCAATG |
| | | GGGCGGTGGCTGCTGTGCTACTGAGTCAGCGC |
| | | ACTGCCCTGAGCAGCACGGACACCTTCCTGCT |
| | | CCACCTGGCTGTAGCCGATGTTCTGCTGGTGT |
| | | TAACTCTTCCATTGTGGGCAGTGGATGCTGCT |
| | | GTCCAGTGGGTTTTCGGCCCTGGCCTCTGCAA |
| | | AGTGGCAGGCGCCTTGTTCAACATCAACTTCT |
| | | ATGCAGGGGCCTTCCTGCTGGCTTGTATAAGC |
| | | TTCGACAGATATCTGAGCATAGTGCACGCCAC |
| | | CCAGATCTACCGCAGGGACCCCCGGGTACGTG |
| | | TAGCCCTCACCTGCATAGTTGTATGGGGTCTC |
| | | TGTCTGCTCTTTGCCCTCCCAGATTTCATCTAC |
| | | CTATCAGCCAACTACGATCAGCGCCTCAATGC |
| | | CACCCATTGCCAGTACAACTTCCCACAGGTGG |
| | | GTCGCACTGCTCTGCGTGTACTGCAGCTAGTG |
| | | GCTGGTTTCCTGCTGCCCCTTCTGGTCATGGC |
| | | CTACTGCTATGCCCATATCCTAGCTGTTCTGCT |
| | | GGTCTCCAGAGGCCAGAGGCGTTTTCGAGCTA |
| | | TGAGGCTAGTGGTAGTGGTGGTGGCAGCCTTT |
| | | GCTGTCTGCTGGACCCCCTATCACCTGGTGGT |
| | | GCTAGTGGATATCCTCATGGATGTGGGAGTTT |
| | | TGGCCCGCAACTGTGGTCGAGAAAGCCACGTG |
| | | GATGTGGCCAAGTCAGTCACCTCGGGCATGGG |
| | | GTACATGCACTGCTGCCTCAATCCGCTGCTCT |
| | | ATGCCTTTGTGGGAGTGAAGTTCAGAGAGCAA |
| | | ATGTGGATGTTGTTCACGCGCCTGGGCCGCTC |
| | | TGACCAGAGAGGGCCCCAGCGGCAGCCGTCAT |
| | | CTTCACGGAGAGAATCATCCTGGTCTGAGACA |
| | | ACTGAGGCCTCCTACCTGGGCTTGTAATAGGA |
| | | GCTCTAGAATAACTTCGTATAGCATACATTATA |
| | | CGAAGTTATAAAGTTAAAAGTTAATGAGAGCAA |
| | | AAATATATAAGGTTGTATTCCATATTTGTTATTTT |
| | | TTCTGTAATAGTTAGAAAAATACATTCGATGGTC |
| | | TATCTACCAGATTATTATGTGTTATAAGGTACTTT |
| | | TTCTCATAATAAACTAGAGTATGAGTAAGATAGT |
| | | GTTTTTCAAAACATATAAATCTAAAATTGATGGA |
| | | TGAGATATACAGCTATTAATTTCGAAAATATATTT |
| | | TAATCTGATAACTTTAAACATGGATTTTTGATGGT |
| | | GGTTTAACGTTTTAAAAAAAGATTTTGTTATTGTA |
| | | GTATATGATAATATTAAAAGATGGATATAAAGAA |
| | | TTTGCTGACTGCATGTACTATTTTTTACATTACTA |
| | | CATTGGCTACGGCAGATATACCTACTCCGCCACC |
| | | AACGGGTCATGTGACAAGGGAGAATATCTTGATA |
| | | AGAGGCATAATCAATGTTGTAATCGGTGTCCACC |
| | | TGGAGAATTTGCCAAGGTTAGATGTAAT |

### Example 8: Chemokine Receptor Sequence Addition

The modified vaccinia virus described in Example 2 is further modified by replacing the gene encoding A52 (VACV094, J2R) with nucleic acid encoding human CXCR3 and a fluorescent reporter. A plasmid transfer vector is generated comprising, in order, with no gaps, an upstream recombination directing sequence B (SEQ ID NO: 70), an open reading frame encoding human CXCR3 isoform 1 (SEQ ID NO: 76), a stop codon, SacI cloning site, and short spacer (SEQ ID NO: 72), a loxP site (SEQ ID NO: 54), a spacer followed by viral promoter driving expression of GFP-pac reporter (SEQ ID NO: 73), a PacI cloning site (TTAATTAA), short spacer B (SEQ ID NO: 57), a loxP site (SEQ ID NO: 54), and a downstream recombination directing sequence B (SEQ ID NO: 74). Reporter-positive viruses are isolated, then treated with a reporter-free transfer vector comprising an upstream recombination-directing sequence B (SEQ ID NO: 70), an open reading frame encoding human CXCR3 isoform 1 (SEQ ID NO: 76), and a second downstream recombination-directing sequence B (SEQ ID NO: 77). Following recombination, the viral genome comprises the incorporated sequence of SEQ ID NO: 78. Selected sequences are shown in Table 10.

TABLE 10
Human CXCR3 recombination sequences.

| SEQ ID | | |
|---|---|---|
| NO: | Description | Nucleic acid sequence |
| 76 | Open reading frame encoding | ATGGTCCTTGAGGTGAGTGACCACCAAGTGCTA |
| | human CXCR3 isoform 1 | AATGACGCCGAGGTTGCCGCCCTCCTGGAGAAC |
| | | TTCAGCTCTTCCTATGACTATGGAGAAAACGAG |
| | | AGTGACTCGTGCTGTACCTCCCCGCCCTGCCCAC |
| | | AGGACTTCAGCCTGAACTTCGACCGGGCCTTCCT |
| | | GCCAGCCCTCTACAGCCTCCTCTTTCTGCTGGGG |
| | | CTGCTGGGCAACGGCGCGGTGGCAGCCGTGCTG |
| | | CTGAGCCGGCGGACAGCCCTGAGCAGCACCGAC |
| | | ACCTTCCTGCTCCACCTAGCTGTAGCAGACACGC |
| | | TGCTGGTGCTGACACTGCCGCTCTGGGCAGTGG |
| | | ACGCTGCCGTCCAGTGGGTCTTTGGCTCTGGCCT |
| | | CTGCAAAGTGGCAGGTGCCCTCTTCAACATCAA |
| | | CTTCTACGCAGGAGCCCTCCTGCTGGCCTGCATC |
| | | AGCTTTGACCGCTACCTGAACATAGTTCATGCCA |
| | | CCCAGCTCTACCGCCGGGGGCCCCCGGCCCGCG |
| | | TGACCCTCACCTGCCTGGCTGTCTGGGGGCTCTG |
| | | CCTGCTTTTCGCCCTCCCAGACTTCATCTTCCTGT |
| | | CGGCCCACCACGACGAGCGCCTCAACGCCACCC |
| | | ACTGCCAATACAACTTCCCACAGGTGGGCCGCA |
| | | CGGCTCTGCGGGTGCTGCAGCTGGTGGCTGGCTT |
| | | TCTGCTGCCCCTGCTGGTCATGGCCTACTGCTAT |
| | | GCCCACATCCTGGCCGTGCTGCTGGTTTCCAGGG |
| | | GCCAGCGGCGCCTGCGGGCCATGCGGCTGGTGG |
| | | TGGTGGTCGTGGTGGCCTTTGCCCTCTGCTGGAC |
| | | CCCCTATCACCTGGTGGTGCTGGTGGACATCCTC |
| | | ATGGACCTGGGCGCTTTGGCCCGCAACTGTGGC |
| | | CGAGAAAGCAGGGTAGACGTGGCCAAGTCGGTC |
| | | ACCTCAGGCCTGGGCTACATGCACTGCTGCCTCA |
| | | ACCCGCTGCTCTATGCCTTTGTAGGGGTCAAGTT |
| | | CCGGGAGCGGATGTGGATGCTGCTCTTGCGCCT |
| | | GGGCTGCCCCAACCAGAGAGGGCTCCAGAGGCA |
| | | GCCATCGTCTTCCCGCCGGGATTCATCCTGGTCT |
| | | GAGACCTCAGAGGCCTCCTACTCGGGCTTGTGA |
| 77 | Second downstream | AAAGTTAAAAGTTAATGAGAGCAAAAATATATA |
| | recombination-directing | AGGTTGTATTCCATATTTGTTATTTTTTCTGTAAT |
| | sequence | AGTTAGAAAAATACATTCGATGGTCTATCTACCA |
| | | GATTATTATGTGTTATAAGGTACTTTTTCTCATA |
| | | ATAAACTAGAGTATGAGTAAGATAGTGTTTTTCA |
| | | AAACATATAAATCTAAAATTGATGGATGAGATA |
| | | TACAGCTATTAATTTCGAAAATATATTTTAATCT |
| | | GATAACTTTAAACATGGATTTTTGATGGTGGTTT |
| | | AACGTTTTAAAAAAAGATTTTGTTATTGTAGTAT |
| | | ATGATAATATTAAAAGATGGATATAAAGAATTT |
| | | GCTGACTGCATGTACTATTTTTTACATTACTACA |
| | | TTGGCTACGGCAGATATACCTACTCCGCCACCAA |
| | | CGGGTCATGTGACAAGGGAGAATATCTTGATAA |
| | | GAGGCATAATCAATGTTGTAATCGGTGTCCACCT |
| | | GGAGAATTTGCCAAGGTTAGATGTAATGGTAAC |
| | | GATAACACAAAATGTGAACGCTGCCCACCTCAT |
| | | ACATATACCACAATCCCAATTATTCTAATGGATG |
| | | TCATCAATGTAGAAAATGCCCAACCGGATCATTT |
| | | GATAAGGTAAAGTGTACCGGAACACAGAACAGT |
| | | AAATGTTCGTGTCTTCCTGGTTGGTATTGTGCTA |
| | | CTGATTCTTCACAGACTGAAGATTGTTGAAATTG |
| | | TGTACCAAAAAGGAGATGTCCATGCGGATACTT |
| | | TGGTGGAATAGATGAACAAGGAAATCCTATTTG |
| | | TAAATCGTGTTGTGTTGGTGAATATTGCGACTAC |
| | | CTACGTAATTATAGACTTGATCCATTTCCTCCAT |
| | | GCAAACTATCTA |
| 78 | Sequence incorporated in | CTCATCAGACATATCGTGTGCATGCGTGGAGTTA |
| | viral genome (non-native | AATTCATATCACGTAGCCATTCTTCCCATGGATG |
| | DNA BOLD; CXCR3 | TTTCCTTTTTTACCAAAGGAAATGCATCATTGAT |
| | transgene UNDERLINED) | TATTCTCCTGTTTGATTTCTCTATCGATGCAGCAC |
| | | CTCTCTTAAGAAGTGTAACCGATAATAATGTTAT |
| | | TATATCTAGACACCAGCGTCTACATGACGAGCTT |
| | | CCGAGTTCCAATTGGTTCAAGTTTTACATAAGTA |
| | | TAAAGTCCGACTATTGTTCTATATTATATATGGT |
| | | TGTTGATGGATCTGTGATGCATGCGATAGCTGAT |
| | | AATAGAACTCACGCAATTATTAGCAAAAATATA |
| | | TTAGACAATACTACGATTAACGATGAGTGTAGA |
| | | TGCTGTTATTTTGAACCACAGATTAGGATTCTTG |
| | | ATAGAGATGAGATGCTCAATGGATCATCGTGTG |
| | | ATATGAACAGACATTGTATTATGATGAATTTACC |
| | | TGATGTAGGCAAATTTGGATCTAGTATGTTGGGG |
| | | AAATATGAACCTGACATGATTAAGATTGCTCTTT |
| | | CGGTGGCTGGTAATTTAATAAGAAATCGAGACT |
| | | ACATTCCCGGGAGACGAGGATATAGCTACTACG |
| | | TTTACGGTATAGCCTCTAGATAATTTTTTTAAGC |
| | | ACGAAATAAAAAACATAATTTTAAACCAATCTA |
| | | TTTCATACTATTTTGTGTGATCACCATGGTCCTT |
| | | GAGGTGAGTGACCACCAAGTGCTAAATGACG |
| | | CCGAGGTTGCCGCCCTCCTGGAGAACTTCAG |
| | | CTCTTCCTATGACTATGGAGAAAACGAGAGTG |
| | | ACTCGTGCTGTACCTCCCCGCCCTGCCCACAG |
| | | GACTTCAGCCTGAACTTCGACCGGGCCTTCCT |
| | | GCCAGCCCTCTACAGCCTCCTCTTTCTGCTGG |
| | | GGCTGCTGGGCAACGGCGCGGTGGCAGCCGT |
| | | GCTGCTGAGCCGGCGGACAGCCCTGAGCAGC |
| | | ACCGACACCTTCCTGCTCCACCTAGCTGTAGC |
| | | AGACACGCTGCTGGTGCTGACACTGCCGCTC |
| | | TGGGCAGTGGACGCTGCCGTCCAGTGGGTCT |
| | | TTGGCTCTGGCCTCTGCAAAGTGGCAGGTGC |
| | | CCTCTTCAACATCAACTTCTACGCAGGAGCCC |
| | | TCCTGCTGGCCTGCATCAGCTTTGACCGCTAC |
| | | CTGAACATAGTTCATGCCACCCAGCTCTACCG |
| | | CCGGGGGCCCCCGGCCCGCGTGACCCTCACC |
| | | TGCCTGGCTGTCTGGGGGCTCTGCCTGCTTTT |
| | | CGCCCTCCCAGACTTCATCTTCCTGTCGGCCC |
| | | ACCACGACGAGCGCCTCAACGCCACCCACTG |
| | | CCAATACAACTTCCCACAGGTGGGCCGCACG |
| | | GCTCTGCGGGTGCTGCAGCTGGTGGCTGGCT |
| | | TTCTGCTGCCCCTGCTGGTCATGGCCTACTGC |
| | | TATGCCCACATCCTGGCCGTGCTGCTGGTTTC |
| | | CAGGGGCCAGCGGCGCCTGCGGGCCATGCGG |
| | | CTGGTGGTGGTGGTCGTGGTGGCCTTTGCCC |
| | | TCTGCTGGACCCCCTATCACCTGGTGGTGCTG |
| | | GTGGACATCCTCATGGACCTGGGCGCTTTGG |
| | | CCCGCAACTGTGGCCGAGAAAGCAGGGTAGA |
| | | CGTGGCCAAGTCGGTCACCTCAGGCCTGGGC |
| | | TACATGCACTGCTGCCTCAACCCGCTGCTCTA |
| | | TGCCTTTGTAGGGGTCAAGTTCCGGGAGCGG |
| | | ATGTGGATGCTGCTCTTGCGCCTGGGCTGCC |
| | | CCAACCAGAGAGGGCTCCAGAGGCAGCCATC |
| | | GTCTTCCCGCCGGGATTCATCCTGGTCTGAGA |
| | | CCTCAGAGGCCTCCTACTCGGGCTTGTGAAA |
| | | AGTTAAAAGTTAATGAGAGCAAAAATATATAAG |
| | | GTTGTATTCCATATTTGTTATTTTTTCTGTAATAG |
| | | TTAGAAAAATACATTCGATGGTCTATCTACCAGA |
| | | TTATTATGTGTTATAAGGTACTTTTTCTCATAATA |
| | | AACTAGAGTATGAGTAAGATAGTGTTTTTCAAA |
| | | ACATATAAATCTAAAATTGATGGATGAGATATA |
| | | CAGCTATTAATTTCGAAAATATATTTTAATCTGA |
| | | TAACTTTAAACATGGATTTTTGATGGTGGTTTAA |
| | | CGTTTTAAAAAAAGATTTTGTTATTGTAGTATAT |
| | | GATAATATTAAAAGATGGATATAAAGAATTTGC |
| | | TGACTGCATGTACTATTTTTTACATTACTACATT |
| | | GGCTACGGCAGATATACCTACTCCGCCACCAAC |
| | | GGGTCATGTGACAAGGGAGAATATCTTGATAAG |
| | | AGGCATAATCAATGTTGTAATCGGTGTCCACCTG |
| | | GAGAATTTGCCAAGGTTAGATGTAATGGTAACG |
| | | ATAACACAAAATGTGAACGCTGCCCACCTCATA |
| | | CATATACCACAATCCCAATTATTCTAATGGATGT |
| | | CATCAATGTAGAAAATGCCCAACCGGATCATTT |
| | | GATAAGGTAAAGTGTACCGGAACACAGAACAGT |
| | | AAATGTTCGTGTCTTCCTGGTTGGTATTGTGCTA |
| | | CTGATTCTTCACAGACTGAAGATTGTTGAAATTG |
| | | TGTACCAAAAAGGAGATGTCCATGCGGATACTT |
| | | TGGTGGAATAGATGAACAAGGAAATCCTATTTG |
| | | TAAATCGTGTTGTGTTGGTGAATATTGCGACTAC |
| | | CTACGTAATTATAGACTTGATCCATTTCCTCCAT |
| | | GCAAACTATCTA |

The foregoing description and accompanying drawings set forth a number of representative embodiments at the present time. Various modifications, additions, and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A composition, wherein the composition comprises:
a vector;
a first exogenous nucleic acid comprising a sequence encoding for a cytokine or a functional variant thereof; and
a second exogenous nucleic acid comprising a sequence encoding for a PD-L1 receptor.

2. The composition of claim 1, wherein the encoded cytokine comprises IL-12 or a functional variant thereof.

3. The composition of claim 2, wherein the IL-12 comprises a sequence having at least 85%, 90%, 95%, or 99% sequence identity to SEQ ID NO: 8 or SEQ ID NO: 5.

4. The composition of claim 2, wherein the IL-12 comprises a sequence of SEQ ID NO: 8 or SEQ ID NO: 5.

5. The composition of claim 1, wherein the encoded PD-L1 receptor comprises a PD-1 dominant negative or a protein that binds to PD-L1.

6. The composition of claim 5, wherein the protein that binds to PD-L1 is an anti-PD-L1 antibody or a soluble variant of PD-1 (SPD-1).

7. The composition of claim 6, wherein the sPD-1 comprises a sequence having at least 85%, 90%, 95%, or 99% sequence identity to SEQ ID NO: 4 or SEQ ID NO: 2.

8. The composition of claim 6, wherein the sPD-1 comprises a sequence of SEQ ID NO: 4 or SEQ ID NO: 2.

9. The composition of claim 8, wherein the sPD-1 comprises one or more substitutions as described by E61V, M70I, Q75F, K78W, K78L, E84F, S87W, A129H, A132L, K135M, according to SEQ ID NO: 4.

10. The composition of claim 1, further comprising at least one promoter region, wherein the at least one promoter region drives expression of the cytokine and the PD-L1 receptor, and wherein the at least one promoter comprises any one of P7.5, P28, P135, SSP, 454, TK promoter, E/L, F7L, H5R, H1L, A1L, J3R, E4L, I1L, I5L, I7L, T7, I2L, FP4b, ATI, P11, PFL1, PH5, L4R, 28 kDa promoter, or any variation or combination thereof.

11. The composition of claim 10, wherein the at least one promoter region comprises a first promoter that drives expression of the cytokine, and a second promoter that drives expression of the PD-L1 receptor.

12. The composition of claim 11, wherein the first promoter comprises a P7.5 promoter, and the second promoter comprises a P28 promoter.

13. The composition of claim 1, wherein the first and the second exogenous nucleic acids comprise a combined sequence of SEQ ID NO: 64 or SEQ ID NO: 59.

14. The composition of claim 1, further comprising a third exogenous nucleic acid comprising a sequence encoding for a chemokine receptor or a functional variant thereof, wherein the chemokine receptor comprises at least one of: a CXC receptor, a CC receptor, a CX3C receptor, an XC receptor, a functional fragment thereof, a functional variant thereof, or any combinations thereof.

15. The composition of claim 14, wherein the chemokine receptor comprises at least one of: CXCR1, CXCR2, CXCR3, CXCR4, CXCR5, CXCR6, CXCR7, CCR1, CCR2, CCR3, CCR4, CCR5, CCR6, CCR7, CCR8, CCR9, CCR10, CCR11, CX3CR1, XCR1, a functional fragment thereof or a functional variant thereof, or any combinations thereof.

16. The composition of claim 15, therein the chemokine receptor comprises an amino acid sequence of any one of SEQ ID NOs: 27-40.

17. The composition of claim 1, wherein the vector comprises a plasmid, a phage, a virus, a cosmid, an artificial chromosome, a liposome, a nanoparticle, or any combination thereof.

18. The composition of claim 17, wherein the virus is an oncolytic virus.

19. The composition of claim 18, wherein the oncolytic virus is a poxvirus, an adeno associated virus, an adenovirus, a reovirus, a lentivirus, a herpes simplex virus, a vesicular stomatitis virus, a mengovirus, a myxoma virus, Newcastle disease virus, measles virus, or polio virus.

20. The composition of claim 19, wherein the poxvirus is a vaccinia virus, and wherein the vaccinia virus is a modified strain of Western Reserve Vaccinia virus (ATCC VR-1354), Vaccinia virus Ankara (ATCC VR-1508), Vaccinia virus Ankara (ATCC VR-1566), Vaccinia virus strain Wyeth (ATCC VR-1536), or Vaccinia virus Wyeth (ATCC VR-325).

21. The composition of claim 14, wherein the first, the second, and the third exogenous nucleic acids are inserted into the viral genome.

22. The composition of claim 18, wherein the oncolytic virus comprises at least one genome modification comprising a mutation or deletion of at least one gene selected from the group consisting of: Thymidine Kinase (TK), F13L, A36R, A34R, A33R, A52R, B5R, B8R, B18R, SPI-1, SPI-2, B15R, VGF, E3L, K3L, A41L, K7R, or NIL, or any combinations thereof.

23. The composition of claim 22, comprising the mutation or deletion of the TK gene.

24. An oncolytic virus, wherein the oncolytic virus comprises:
an insertion at a TK gene locus comprising, in 5′ to 3′ order:
a first promoter region comprising a sequence of SEQ ID NO: 44, wherein the promoter is P7.5;
a first region comprising a sequence of SEQ ID NO: 8 encoding IL-12;
a second promoter region comprising a sequence of SEQ ID NO: 41, wherein the promoter is P28; and
a second region comprising a sequence of SEQ ID NO: 4 encoding a PD-1 variant.

25. A pharmaceutical composition, wherein the pharmaceutical composition comprises:
the composition of claim 1; and
a pharmaceutically acceptable excipient.

26. A method for treatment of cancer comprising administering to a subject having cancer the pharmaceutical composition of claim 25 in an amount sufficient for treatment of a cancer.


SEQUENCE LISTING (78 sequences)

SEQ ID NO: 1 - protein, 288 aa, Mus sp.
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 2 - protein, 168 aa, Mus sp.
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQG

SEQ ID NO: 3 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 4 - protein, 170 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 5 - protein, 562 aa, Mus sp.
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSGGGGSASATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 6 - protein, 215 aa, Mus sp.
MCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 7 - protein, 335 aa, Mus sp.
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 8 - protein, 602 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSASWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 9 - protein, 252 aa, Homo sapiens
WPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 10 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 11 - protein, 22 aa, synthetic construct
GGGGSGGGGSGGGGSGGGGSAS

SEQ ID NO: 12 - protein, 15 aa, synthetic construct
  VARIANT 1..15: note = This sequence may encompass 1-3 GGGGS repeating units
GGGGSGGGGSGGGGS

SEQ ID NO: 13 - protein, 8 aa, synthetic construct
  VARIANT 1..8: note = This sequence may encompass 6-8 residues
GGGGGGGG

SEQ ID NO: 14 - protein, 15 aa, synthetic construct
  VARIANT 1..15: note = This sequence may encompass 1-3 EAAAK repeating units
EAAAKEAAAKEAAAK

SEQ ID NO: 15 - protein, 46 aa, synthetic construct
AEAAAKEAAAKEAAAKEAAAKALEAEAAAKEAAAKEAAAKEAAAKA

SEQ ID NO: 16 - protein, 5 aa, synthetic construct
PAPAP

SEQ ID NO: 17 - protein, 17 aa, synthetic construct
VSQTSKLTRAETVFPDV

SEQ ID NO: 18 - protein, 6 aa, synthetic construct
PLGLWA

SEQ ID NO: 19 - protein, 6 aa, synthetic construct
RVLAEA

SEQ ID NO: 20 - protein, 10 aa, synthetic construct
EDVVCCSMSY

SEQ ID NO: 21 - protein, 8 aa, synthetic construct
GGIEGRGS

SEQ ID NO: 22 - protein, 10 aa, synthetic construct
TRHRQPRGWE

SEQ ID NO: 23 - protein, 10 aa, synthetic construct
AGNRVRRSVG

SEQ ID NO: 24 - protein, 9 aa, synthetic construct
RRRRRRRRR

SEQ ID NO: 25 - protein, 4 aa, synthetic construct
GFLG

SEQ ID NO: 26 - protein, 17 aa, synthetic construct
GGGGSSPLGLWAGGGGS

SEQ ID NO: 27 - protein, 367 aa, Mus sp.
MYLEVSERQVLDASDFAFLLENSTSPYDYGENESDFSDSPPCPQDFSLNFDRTFLPALYSLLFLLGLLGNGAVAAVLLSQRTALSSTDTFLLHLAVADVLLVLTLPLWAVDAAVQWVFGPGLCKVAGALFNINFYAGAFLLACISFDRYLSIVHATQIYRRDPRVRVALTCIVVWGLCLLFALPDFIYLSANYDQRLNATHCQYNFPQVGRTALRVLQLVAGFLLPLLVMAYCYAHILAVLLVSRGQRRFRAMRLVVVVVAAFAVCWTPYHLVVLVDILMDVGVLARNCGRESHVDVAKSVTSGMGYMHCCLNPLLYAFVGVKFREQMWMLFTRLGRSDQRGPQRQPSSSRRESSWSETTEASYLGL

SEQ ID NO: 28 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 29 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 30 - protein, 374 aa, unidentified; identical to SEQ ID NO: 29

SEQ ID NO: 31 - protein, 357 aa, Homo sapiens
MEDFNMESDSFEDFWKGEDLSNYSYSSTLPPFLLDAAPCEPESLEINKYFWIIYALVFLLSLLGNSLVMLVILYSRVGRSVTDVYLLNLALADLLFALTLPIWAASKVNGWIFGTFLCKWSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAWLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 32 - protein, 374 aa, Homo sapiens; identical to SEQ ID NO: 29

SEQ ID NO: 33 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVAVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 34 - protein, 359 aa, Mus sp.
MEPISVSIYTSDNYSEEVGSGDYDSNKEPCFRDENVHFNRIFLPTIYFIIFLTGIVGNGLVILVMGYQKKLRSMTDKYRLHLSVADLLFVITLPFWAVDAMADWYFGKFLCKAVHIIYTVNLYSSVLILAFISLDRYLAIVHATNSQRPRKLLAEKAVAVGVWIPALLLTIPDFIFADVSQGDISQGDDRYICDRLYPDSLWMVVFQFQHIMVGLVLPGIVILSCYCIIISKLSHSKGHQKRKALKTTVILILAFFACWLPYYVGISIDSFILLGVIKQGCDFESIVHKWISITEALAFFHCCLNPILYAFLGAKFKSSAQHALNSMSRGSSLKILSKGKRGGHSSVSTESESSSFHSS

SEQ ID NO: 35 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 36 - protein, 352 aa, Homo sapiens; identical to SEQ ID NO: 33

SEQ ID NO: 37 - protein, 359 aa, Mus sp.; identical to SEQ ID NO: 34

SEQ ID NO: 38 - protein, 352 aa, Homo sapiens; identical to SEQ ID NO: 35

SEQ ID NO: 39 - protein, 90 aa, Homo sapiens
MKVSAAALAVILIATALCAPASASPYSSDTTPCCFAYIARPLPRAHIKEYFYTSGKCSNPAWFVTRKNRQVCANPEKKWVREYINSLEMS

SEQ ID NO: 40 - protein, 349 aa, Homo sapiens
MDYQVSSPIYDINYYTSEPCQKINVKQIAARLLPPLYSLVFIFGFVGNMLVILILINCKRLKSMTDIYLLNLAISDLFFLLTVPFWAHYAAAQWDFGNTMCQLLTGLYFIGFFSGIFFIILLTIDRYLAWHAVFALKARTVTFGWTSVITWWAVFASLPGIIFTRSQKEGLHYTCSSHFPYSQYQFWKNFQTLKIVILGLVLPLLVMVICYSGILKTLLRCRNEKKRHRAVRLIFTIMIVYFLFWAPYNIVLLLNTFQEFFGLNNCSSSNRLDQAMQVTETLGMTHCCINPIIYAFVGEKFRNYLLVFFQKHIAKRFCKCCSIFQQEAPERASSVYTRSTGEQEISVGL

SEQ ID NO: 41 - DNA, 37 bp, unidentified
  source 1..37: mol_type = unassigned DNA
aagctcacaaaaaaaacttctctaaatagattcgagc

SEQ ID NO: 42 - DNA, 64 bp, unidentified
  source 1..64: mol_type = unassigned DNA
ggatcataaaaatatagtagaatttcattttgtttttttctatgctataaatagcttctcgagc

SEQ ID NO: 43 - DNA, 37 bp, Vaccinia virus
  source 1..37: mol_type = genomic DNA
cctgcagggcgtcctaaaattaaaacaaatgatataa

SEQ ID NO: 44 - DNA, 130 bp, Vaccinia virus
  source 1..130: mol_type = genomic DNA
tcactaattccaaacccacccgctttttatagtaagtttttcacccataaataataaatacaataattaatttctcgtaaaagtagaaaatatattctaatttattgcacggtaaggaagtagatcataa

SEQ ID NO: 45 - DNA, 204 bp, Vaccinia virus
  source 1..204: mol_type = genomic DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 46 - DNA, 511 bp, Homo sapiens
  source 1..511: mol_type = genomic DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 47 - DNA, 1000 bp, synthetic construct
  source 1..1000: mol_type = other DNA
atttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatc

SEQ ID NO: 48 - DNA, 1689 bp, Mus sp.
  source 1..1689: mol_type = genomic DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccatgcagggtccgatccggaggtggcggatccggcggaggtggctcaggtggcggtggatcaggaggaggtggatcagctagcgccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgaccatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgacgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagttccgcctaa

SEQ ID NO: 49 - DNA, 1005 bp, Mus sp.
  source 1..1005: mol_type = genomic DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccatgcagggtccgatcc

SEQ ID NO: 50 - DNA, 66 bp, synthetic construct
  source 1..66: mol_type = other DNA
ggaggtggcggatccggcggaggtggctcaggtggcggtggatcaggaggaggtggatcagctagc

SEQ ID NO: 51 - DNA, 618 bp, Mus sp.
  source 1..618: mol_type = genomic DNA
gccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgaccatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgacgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagttccgcctaa

SEQ ID NO: 52 - DNA, 19 bp, synthetic construct
  source 1..19: mol_type = other DNA
gtcgactgagtgaccgtga

SEQ ID NO: 53 - DNA, 507 bp, synthetic construct
  source 1..507: mol_type = other DNA
atgtgggtccggcaagtaccctggtcattcacttgggctgtgctgcagttgagctggcaatcagggtggcttctagaggtccccaatgggccctggaggtccctcaccttctacccagcctggctcacagtgtcagagggagcaaatgccaccttcacctgcagcttgtccaactggtcggaggatcttatgctgaactggaaccgcctgagtcccagcaaccagactgaaaaacaggccgccttctgtaatggtttgagccaacccgtccaggatgcccgcttccagatcatacagctgcccaacaggcatgacttccacatgaacatccttgacacacggcgcaatgacagtggcatctacctctgtggggccatctccctgcaccccaaggcaaaaatcgaggagagccctggagcagagctagtggtaacagagagaatcctggagacctcaacaagatatcccagcccctcgcccaaaccagaaggccggtttcaaggctag

SEQ ID NO: 54 - DNA, 34 bp, synthetic construct
  source 1..34: mol_type = other DNA
ataacttcgtatagcatacattatacgaagttat

SEQ ID NO: 55 - DNA, 12 bp, synthetic construct
  source 1..12: mol_type = other DNA
aattctgcagat

SEQ ID NO: 56 - DNA, 717 bp, synthetic construct
  source 1..717: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtcccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataa

SEQ ID NO: 57 - DNA, 12 bp, synthetic construct
  source 1..12: mol_type = other DNA
aaggccgctcga

SEQ ID NO: 58 - DNA, 997 bp, synthetic construct
  source 1..997: mol_type = other DNA
tattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacg

SEQ ID NO: 59 - DNA, 4439 bp, synthetic construct
  source 1..4439: mol_type = other DNA
atttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatccctgcaggtcactaattccaaacccacccgctttttatagtaagtttttcacccataaataataaatacaataattaatttctcgtaaaagtagaaaatatattctaatttattgcacggtaaggaagtagatcataaggtaccatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccatgcagggtccgatccggaggtggcggatccggcggaggtggctcaggtggcggtggatcaggaggaggtggatcagctagcgccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgaccatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgacgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagttccgcctaagtcgactgagtgaccgtgaaagctcacaaaaaaaacttctctaaatagattcgagcactagtatgtgggtccggcaagtaccctggtcattcacttgggctgtgctgcagttgagctggcaatcagggtggcttctagaggtccccaatgggccctggaggtccctcaccttctacccagcctggctcacagtgtcagagggagcaaatgccaccttcacctgcagcttgtccaactggtcggaggatcttatgctgaactggaaccgcctgagtcccagcaaccagactgaaaaacaggccgccttctgtaatggtttgagccaacccgtccaggatgcccgcttccagatcatacagctgcccaacaggcatgacttccacatgaacatccttgacacacggcgcaatgacagtggcatctacctctgtggggccatctccctgcaccccaaggcaaaaatcgaggagagccctggagcagagctagtggtaacagagagaatcctggagacctcaacaagatatcccagcccctcgcccaaaccagaaggccggtttcaaggctaggagctcataacttcgtatagcatacattatacgaagttattattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacg

SEQ ID NO: 60 - DNA, 1809 bp, Homo sapiens
  source 1..1809: mol_type = genomic DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggaggtggcggatccggcggaggtggctcaggtggcggtggatcaggaggaggtggatcagctagctggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 61 - DNA, 984 bp, Homo sapiens
  source 1..984: mol_type = genomic DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 62 - DNA, 759 bp, Homo sapiens
  source 1..759: mol_type = genomic DNA
tggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 63 - DNA, 513 bp, synthetic construct
  source 1..513: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtatcgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagaactcagggtgacagagagaagggcagaagtgcccacagcccaccccagcccctcacccaggccagccggccagttccaaaccctggtgtaa

SEQ ID NO: 64 - DNA, 4565 bp, synthetic construct
  source 1..4565: mol_type = other DNA
atttacggattcaccaataaaaataaactagagaaacttagtactaataaggaactagaatcgtatagttctagccctcttcaagaacccattaggttaaatgattttctgggactattggaatgtgttaaaaagaatattcctctaacagatattccgacaaaggattgattactataaatggagaatgttcctaatgtatactttaatcctgtgtttatagagcccacgtttaaacattctttattaagtgtttataaacacagattaatagttttatttgaagtattcgttgtattcattctaatatatgtattttttagatctgaattaaatatgttcttcatgcctaaacgaaaaatacccgatcctattgatagattacgacgtgctaatctagcgtgtgaagacgataaattaatgatctatggattaccatggatgacaactcaaacatctgcgttatcaataaatagtaaaccgatagtgtataaagattgtgcaaagcttttgcgatcaataaatggatcacaaccagtatctcttaacgatgttcttcgcagatgatgattcattttttaagtatttggctagtcaagatgatgaatcttcattatctgatatattgcaaatcactcaatatctagactttctgttattattattgatccaatcaaaaaataaattagaagccgtgggtcattgttatgaatctctttcagaggaatacagacaattgacaaaattcacagactttcaagattttaaaaaactgtttaacaaggtccctattgttacagatggaagggtcaaacttaataaaggatatttgttcgactttgtgattagtttgatgcgattcaaaaaagaatcctctctagctaccaccgcaatagatcctgttagatacatagatcctcgtcgcaatatcgcattttctaacgtgatggatatattaaagtcgaataaagtgaacaataattaattctttattgtcatccctgcaggtcactaattccaaacccacccgctttttatagtaagtttttcacccataaataataaatacaataattaatttctcgtaaaagtagaaaatatattctaatttattgcacggtaaggaagtagatcataaggtaccatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggaggtggcggatccggcggaggtggctcaggtggcggtggatcaggaggaggtggatcagctagctggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaagtcgactgagtgaccgtgaaagctcacaaaaaaaacttctctaaatagattcgagcactagtatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtatcgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagaactcagggtgacagagagaagggcagaagtgcccacagcccaccccagcccctcacccaggccagccggccagttccaaaccctggtgtaagagctcataacttcgtatagcatacattatacgaagttattattatattttttatctaaaaaactaaaaataaacattgattaaattttaatataatacttaaaaatggatgttgtgtcgttagataaaccgtttatgtattttgaggaaattgataatgagttagattacgaaccagaaagtgcaaatgaggtcgcaaaaaaactgccgtatcaaggacagttaaaactattactaggagaattattttttcttagtaagttacagcgacacggtatattagatggtgccaccgtagtgtatataggatctgctcccggtacacatatacgttatttgagagatcatttctataatttaggagtgatcatcaaatggatgctaattgacggccgccatcatgatcctattttaaatggattgcgtgatgtgactctagtgactcggttcgttgatgaggaatatctacgatccatcaaaaaacaactgcatccttctaagattattttaatttctgatgtgagatccaaacgaggaggaaatgaacctagtacggcggatttactaagtaattacgctctacaaaatgtcatgattagtattttaaaccccgtggcgtctagtcttaaatggagatgcccgtttccagatcaatggatcaaggacttttatatcccacacggtaataaaatgttacaaccttttgctccttcatattcagctgaaatgagattattaagtatttataccggtgagaacatgagactgactcgagttaccaaatcagacgctgtaaattatgaaaaaaagatgtactaccttaataagatcgtccgtaacaaagtagttgttaactttgattatcctaatcaggaatatgactattttcacatgtactttatgctgaggaccgtgtactgcaataaaacatttcctactactaaagcaaaggtactatttctacaacaatctatatttcgtttcttaaatattccaacaacatcaactgaaaaagttagtcatgaaccaatacaacg

SEQ ID NO: 65 - DNA, 1000 bp, synthetic construct
  source 1..1000: mol_type = other DNA
gtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataattatttcaatatactagttaaaattttaagattttaaatgtataaaaaactaataacgtttttatttgtaataggtgcattagcatcctattcgaataatgagtacactccgtttaataaactgagtgtaaaactctatatagatggagtagataatatagaaaattcatatactgatgataataatgaattggtgttaaattttaaagagtacacaatttctattattacagagtcatgcgacgtcggatttgattccatagatatagatgttataaacgactataaaattattgatatgtataccattgactcgtctactattcaacgcagaggtcacacgtgtagaatatctaccaaattatcatgccattatgataagtacccttatattcacaaatatgatggtgatgagcgacaatattctattactgcagagggaaaatgctataaaggaataaaatatgaaataagtatgatcaacgatgatactctattgagaaaacatactcttaaaattggatctacttatatatttgatcgtcatggacatagtaatacatattattcaaaatatgatttttaaaaatttaaaatatattatcacttcagtgacagtagtcaaataacaaacaacacc

SEQ ID NO: 66 - DNA, 20 bp, synthetic construct
  source 1..20: mol_type = other DNA
cgaagttataattctgcaga

SEQ ID NO: 67 - DNA, 717 bp, synthetic construct
  source 1..717: mol_type = other DNA
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtcccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatag

SEQ ID NO: 68 - DNA, 867 bp, synthetic construct
  source 1..867: mol_type = other DNA
ctaatttttattaatgatacaaaaacgaaataaaactgcatattatacactggttaacgcccttataggctctaaccattttcaagatgaggtccctgattatagtccttctgttcccctctatcatctactccatgtctattagacaatgtgagaagactgaagaggaaacatggggattgaaaatagggttgtgtataattgccaaagatttctatcccgaaagaactgattgcagtgttcatctcccaactgcaagtgaaggattgataactgaaggcaatggattcagggatatacgaaacaccgataaattataaaaaaagcaatgtgtccgctgtttccgttaataatactattttcgtaactggcggattattcataaataactctaatagcacgatcgtggttaacaatatggaaaaacttgacatttataaagacaaacaatggtcgattatagaaatgcctatggctagggtatatcacggcatcgactcgacatttggaatgttatattttgccggaggtctatccgttaccgaacaatatggtaatttagagaaaaacaacgagatatcttgttacaatcctagaacgaataagtggtttgatatttcatatactatttataagatatccatatcatcattgtgtaaactaaataacgtcttctatgtatttagtaaggacattggatatgtggaaaagtatgatggtgcatggaagttagtacatgatcgtctccccgctataaaggcattatcaacttctccttattgattgaaaatgaaaatataaatagtttttatgtatagcagtattaccctatagttttattgcttactactaacatggatacagatg

SEQ ID NO: 69 - DNA, 1867 bp, synthetic construct
  source 1..1867: mol_type = other DNA
gtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataattatttcaatatactagttaaaattttaagattttaaatgtataaaaaactaataacgtttttatttgtaataggtgcattagcatcctattcgaataatgagtacactccgtttaataaactgagtgtaaaactctatatagatggagtagataatatagaaaattcatatactgatgataataatgaattggtgttaaattttaaagagtacacaatttctattattacagagtcatgcgacgtcggatttgattccatagatatagatgttataaacgactataaaattattgatatgtataccattgactcgtctactattcaacgcagaggtcacacgtgtagaatatctaccaaattatcatgccattatgataagtacccttatattcacaaatatgatggtgatgagcgacaatattctattactgcagagggaaaatgctataaaggaataaaatatgaaataagtatgatcaacgatgatactctattgagaaaacatactcttaaaattggatctacttatatatttgatcgtcatggacatagtaatacatattattcaaaatatgatttttaaaaatttaaaatatattatcacttcagtgacagtagtcaaataacaaacaacaccctaatttttattaatgatacaaaaacgaaataaaactgcatattatacactggttaacgcccttataggctctaaccattttcaagatgaggtccctgattatagtccttctgttcccctctatcatctactccatgtctattagacaatgtgagaagactgaagaggaaacatggggattgaaaatagggttgtgtataattgccaaagatttctatcccgaaagaactgattgcagtgttcatctcccaactgcaagtgaaggattgataactgaaggcaatggattcagggatatacgaaacaccgataaattataaaaaaagcaatgtgtccgctgtttccgttaataatactattttcgtaactggcggattattcataaataactctaatagcacgatcgtggttaacaatatggaaaaacttgacatttataaagacaaacaatggtcgattatagaaatgcctatggctagggtatatcacggcatcgactcgacatttggaatgttatattttgccggaggtctatccgttaccgaacaatatggtaatttagagaaaaacaacgagatatcttgttacaatcctagaacgaataagtggtttgatatttcatatactatttataagatatccatatcatcattgtgtaaactaaataacgtcttctatgtatttagtaaggacattggatatgtggaaaagtatgatggtgcatggaagttagtacatgatcgtctccccgctataaaggcattatcaacttctccttattgattgaaaatgaaaatataaatagtttttatgtatagcagtattaccctatagttttattgcttactactaacatggatacagatg

SEQ ID NO: 70 - DNA, 700 bp, synthetic construct
  source 1..700: mol_type = other DNA
ctcatcagacatatcgtgtgcatgcgtggagttaaattcatatcacgtagccattcttcccatggatgtttccttttttaccaaaggaaatgcatcattgattattctcctgtttgatttctctatcgatgcagcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcgatagctgataatagaactcacgcaattattagcaaaaatatattagacaatactacgattaacgatgagtgtagatgctgttattttgaaccacagattaggattcttgatagagatgagatgctcaatggatcatcgtgtgatatgaacagacattgtattatgatgaatttacctgatgtaggcaaatttggatctagtatgttggggaaatatgaacctgacatgattaagattgctctttcggtggctggtaatttaataagaaatcgagactacattcccgggagacgaggatatagctactacgtttacggtatagcctctagataatttttttaagcacgaaataaaaaacataattttaaaccaatctatttcatactattttgtgtgatcacc

SEQ ID NO: 71 - DNA, 1104 bp, synthetic construct
  source 1..1104: mol_type = other DNA
atgtaccttgaggttagtgaacgtcaagtgctagatgcctcggactttgcctttcttctggaaaacagcacctctccctacgattatggggaaaacgagagcgacttctctgactccccgccctgcccacaggatttcagcctgaactttgacagaaccttcctgccagccctctacagcctcctcttcttgctggggctgctaggcaatggggcggtggctgctgtgctactgagtcagcgcactgccctgagcagcacggacaccttcctgctccacctggctgtagccgatgttctgctggtgttaactcttccattgtgggcagtggatgctgctgtccagtgggttttcggccctggcctctgcaaagtggcaggcgccttgttcaacatcaacttctatgcaggggccttcctgctggcttgtataagcttcgacagatatctgagcatagtgcacgccacccagatctaccgcagggacccccgggtacgtgtagccctcacctgcatagttgtatggggtctctgtctgctctttgccctcccagatttcatctacctatcagccaactacgatcagcgcctcaatgccacccattgccagtacaacttcccacaggtgggtcgcactgctctgcgtgtactgcagctagtggctggtttcctgctgccccttctggtcatggcctactgctatgcccatatcctagctgttctgctggtctccagaggccagaggcgttttcgagctatgaggctagtggtagtggtggtggcagcctttgctgtctgctggaccccctatcacctggtggtgctagtggatatcctcatggatgtgggagttttggcccgcaactgtggtcgagaaagccacgtggatgtggccaagtcagtcacctcgggcatggggtacatgcactgctgcctcaatccgctgctctatgcctttgtgggagtgaagttcagagagcaaatgtggatgttgttcacgcgcctgggccgctctgaccagagagggccccagcggcagccgtcatcttcacggagagaatcatcctggtctgagacaactgaggcctcctacctgggcttgtaa

SEQ ID NO: 72 - DNA, 13 bp, synthetic construct
  source 1..13: mol_type = other DNA
taggagctctaga

SEQ ID NO: 73 - DNA, 1399 bp, synthetic construct
  source 1..1399: mol_type = other DNA
aattctgcagatggatcataaaaatatagtagaatttcattttgtttttttctatgctataaatagcttctcgagcatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtcccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaaggaggctcaggaaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctga

SEQ ID NO: 74 - DNA, 500 bp, synthetic construct
  source 1..500: mol_type = other DNA
aaagttaaaagttaatgagagcaaaaatatataaggttgtattccatatttgttattttttctgtaatagttagaaaaatacattcgatggtctatctaccagattattatgtgttataaggtactttttctcataataaactagagtatgagtaagatagtgtttttcaaaacatataaatctaaaattgatggatgagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaat

SEQ ID NO: 75 - DNA, 2351 bp, synthetic construct
  source 1..2351: mol_type = other DNA
ctcatcagacatatcgtgtgcatgcgtggagttaaattcatatcacgtagccattcttcccatggatgtttccttttttaccaaaggaaatgcatcattgattattctcctgtttgatttctctatcgatgcagcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcgatagctgataatagaactcacgcaattattagcaaaaatatattagacaatactacgattaacgatgagtgtagatgctgttattttgaaccacagattaggattcttgatagagatgagatgctcaatggatcatcgtgtgatatgaacagacattgtattatgatgaatttacctgatgtaggcaaatttggatctagtatgttggggaaatatgaacctgacatgattaagattgctctttcggtggctggtaatttaataagaaatcgagactacattcccgggagacgaggatatagctactacgtttacggtatagcctctagataatttttttaagcacgaaataaaaaacataattttaaaccaatctatttcatactattttgtgtgatcaccatgtaccttgaggttagtgaacgtcaagtgctagatgcctcggactttgcctttcttctggaaaacagcacctctccctacgattatggggaaaacgagagcgacttctctgactccccgccctgcccacaggatttcagcctgaactttgacagaaccttcctgccagccctctacagcctcctcttcttgctggggctgctaggcaatggggcggtggctgctgtgctactgagtcagcgcactgccctgagcagcacggacaccttcctgctccacctggctgtagccgatgttctgctggtgttaactcttccattgtgggcagtggatgctgctgtccagtgggttttcggccctggcctctgcaaagtggcaggcgccttgttcaacatcaacttctatgcaggggccttcctgctggcttgtataagcttcgacagatatctgagcatagtgcacgccacccagatctaccgcagggacccccgggtacgtgtagccctcacctgcatagttgtatggggtctctgtctgctctttgccctcccagatttcatctacctatcagccaactacgatcagcgcctcaatgccacccattgccagtacaacttcccacaggtgggtcgcactgctctgcgtgtactgcagctagtggctggtttcctgctgccccttctggtcatggcctactgctatgcccatatcctagctgttctgctggtctccagaggccagaggcgttttcgagctatgaggctagtggtagtggtggtggcagcctttgctgtctgctggaccccctatcacctggtggtgctagtggatatcctcatggatgtgggagttttggcccgcaactgtggtcgagaaagccacgtggatgtggccaagtcagtcacctcgggcatggggtacatgcactgctgcctcaatccgctgctctatgcctttgtgggagtgaagttcagagagcaaatgtggatgttgttcacgcgcctgggccgctctgaccagagagggccccagcggcagccgtcatcttcacggagagaatcatcctggtctgagacaactgaggcctcctacctgggcttgtaataggagctctagaataacttcgtatagcatacattatacgaagttataaagttaaaagttaatgagagcaaaaatatataaggttgtattccatatttgttattttttctgtaatagttagaaaaatacattcgatggtctatctaccagattattatgtgttataaggtactttttctcataataaactagagtatgagtaagatagtgtttttcaaaacatataaatctaaaattgatggatgagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaat

SEQ ID NO: 76 - DNA, 1107 bp, synthetic construct
  source 1..1107: mol_type = other DNA
atggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtga

SEQ ID NO: 77 - DNA, 854 bp, synthetic construct
  source 1..854: mol_type = other DNA
aaagttaaaagttaatgagagcaaaaatatataaggttgtattccatatttgttattttttctgtaatagttagaaaaatacattcgatggtctatctaccagattattatgtgttataaggtactttttctcataataaactagagtatgagtaagatagtgtttttcaaaacatataaatctaaaattgatggatgagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaatggtaacgataacacaaaatgtgaacgctgcccacctcatacatataccacaatcccaattattctaatggatgtcatcaatgtagaaaatgcccaaccggatcatttgataaggtaaagtgtaccggaacacagaacagtaaatgttcgtgtcttcctggttggtattgtgctactgattcttcacagactgaagattgttgaaattgtgtaccaaaaaggagatgtccatgcggatactttggtggaatagatgaacaaggaaatcctatttgtaaatcgtgttgtgttggtgaatattgcgactacctacgtaattatagacttgatccatttcctccatgcaaactatcta

SEQ ID NO: 78 - DNA, 2661 bp, synthetic construct
  source 1..2661: mol_type = other DNA
ctcatcagacatatcgtgtgcatgcgtggagttaaattcatatcacgtagccattcttcccatggatgtttccttttttaccaaaggaaatgcatcattgattattctcctgtttgatttctctatcgatgcagcacctctcttaagaagtgtaaccgataataatgttattatatctagacaccagcgtctacatgacgagcttccgagttccaattggttcaagttttacataagtataaagtccgactattgttctatattatatatggttgttgatggatctgtgatgcatgcgatagctgataatagaactcacgcaattattagcaaaaatatattagacaatactacgattaacgatgagtgtagatgctgttattttgaaccacagattaggattcttgatagagatgagatgctcaatggatcatcgtgtgatatgaacagacattgtattatgatgaatttacctgatgtaggcaaatttggatctagtatgttggggaaatatgaacctgacatgattaagattgctctttcggtggctggtaatttaataagaaatcgagactacattcccgggagacgaggatatagctactacgtttacggtatagcctctagataatttttttaagcacgaaataaaaaacataattttaaaccaatctatttcatactattttgtgtgatcaccatggtccttgaggtgagtgaccaccaagtgctaaatgacgccgaggttgccgccctcctggagaacttcagctcttcctatgactatggagaaaacgagagtgactcgtgctgtacctccccgccctgcccacaggacttcagcctgaacttcgaccgggccttcctgccagccctctacagcctcctctttctgctggggctgctgggcaacggcgcggtggcagccgtgctgctgagccggcggacagccctgagcagcaccgacaccttcctgctccacctagctgtagcagacacgctgctggtgctgacactgccgctctgggcagtggacgctgccgtccagtgggtctttggctctggcctctgcaaagtggcaggtgccctcttcaacatcaacttctacgcaggagccctcctgctggcctgcatcagctttgaccgctacctgaacatagttcatgccacccagctctaccgccgggggcccccggcccgcgtgaccctcacctgcctggctgtctgggggctctgcctgcttttcgccctcccagacttcatcttcctgtcggcccaccacgacgagcgcctcaacgccacccactgccaatacaacttcccacaggtgggccgcacggctctgcgggtgctgcagctggtggctggctttctgctgcccctgctggtcatggcctactgctatgcccacatcctggccgtgctgctggtttccaggggccagcggcgcctgcgggccatgcggctggtggtggtggtcgtggtggcctttgccctctgctggaccccctatcacctggtggtgctggtggacatcctcatggacctgggcgctttggcccgcaactgtggccgagaaagcagggtagacgtggccaagtcggtcacctcaggcctgggctacatgcactgctgcctcaacccgctgctctatgcctttgtaggggtcaagttccgggagcggatgtggatgctgctcttgcgcctgggctgccccaaccagagagggctccagaggcagccatcgtcttcccgccgggattcatcctggtctgagacctcagaggcctcctactcgggcttgtgaaaagttaaaagttaatgagagcaaaaatatataaggttgtattccatatttgttattttttctgtaatagttagaaaaatacattcgatggtctatctaccagattattatgtgttataaggtactttttctcataataaactagagtatgagtaagatagtgtttttcaaaacatataaatctaaaattgatggatgagatatacagctattaatttcgaaaatatattttaatctgataactttaaacatggatttttgatggtggtttaacgttttaaaaaaagattttgttattgtagtatatgataatattaaaagatggatataaagaatttgctgactgcatgtactattttttacattactacattggctacggcagatatacctactccgccaccaacgggtcatgtgacaagggagaatatcttgataagaggcataatcaatgttgtaatcggtgtccacctggagaatttgccaaggttagatgtaatggtaacgataacacaaaatgtgaacgctgcccacctcatacatataccacaatcccaattattctaatggatgtcatcaatgtagaaaatgcccaaccggatcatttgataaggtaaagtgtaccggaacacagaacagtaaatgttcgtgtcttcctggttggtattgtgctactgattcttcacagactgaagattgttgaaattgtgtaccaaaaaggagatgtccatgcggatactttggtggaatagatgaacaaggaaatcctatttgtaaatcgtgttgtgttggtgaatattgcgactacctacgtaattatagacttgatccatttcctccatgcaaactatcta